(19) United States
(12) Patent Application Publication
(10) Pub. No.: US 2025/0206704 A1
(43) Pub. Date: Jun. 26, 2025

(54) ENGINEERED IONOPHORES FOR TRANSPORT OF METAL IONS

(71) Applicants: The Brigham and Women's Hospital, Inc., Boston, MA (US); The Broad Institute, Inc., Cambridge, MA (US)

(72) Inventors: Amit Choudhary, Boston, MA (US); Kei Yamada, Cambridge, MA (US); Arghya Deb, Cambridge, MA (US); Veronika Shoba, Cambridge, MA (US); Basudeb Maji, Cambridge, MA (US)

(21) Appl. No.: 18/850,225

(22) PCT Filed: Mar. 24, 2023
(86) PCT No.: PCT/US2023/016208

§ 371 Date: Sep. 24, 2024

Related U.S. Application Data
(60) Provisional application No. 63/323,488, filed on Mar. 24, 2022.

(51) Int. Cl. C07D 213/38; A61K 31/444; B01D 69/02; B01D 69/14; G01N 27/333

(52) U.S. Cl. CPC C07D 213/38; A61K 31/444; B01D 69/02; B01D 69/142; G01N 27/3335; B01D 2325/38; B01D 2325/42

(57) ABSTRACT

Engineered ionophores capable of increased metal ion transport across hydrophobic membranes and/or reduced metal ion binding affinity, pharmaceutical compositions thereof, kits thereof, ion-selective membrane devices thereof, and methods of use thereof. Hydrophobic membranes can be biological, e.g., cell membranes, or nonbiological, e.g., ion-selective membranes. Engineered ionophores can comprise a metal ion chelator group comprising: a polar binding site having binding atoms to form a metal ion chelate complex; and one or more shielding group(s) in proximity to the binding atoms. Shielding groups can increase the hydrophobic membrane permeability and reduce the binding affinity of the chelate complex. Methods of use can comprise contacting said membranes with said engineered ionophores, metal ion chelate complexes thereof, pharmaceutical compositions thereof, the components of kits thereof, or the like, or any combination thereof. Methods of treatment comprise administering the same to a human or non-human animal, plant, or part thereof, e.g. cells.

### CROSS-REFERENCE TO RELATED APPLICATIONS

This application claims the benefit of U.S. Provisional Application No. 63/323,488 filed Mar. 24, 2022. The entire contents of the above-identified applications are hereby fully incorporated herein by reference.

### STATEMENT REGARDING FEDERALLY SPONSORED RESEARCH

This invention was made with government support under Grant No. (s) DK116255 and DK113597 awarded by the National Institutes of Health. The government has certain rights in the invention.

### TECHNICAL FIELD

The subject matter disclosed herein is generally directed to engineered ionophores capable of transporting metal ions across biological membranes for administration as therapeutics and across nonbiological membranes for use in ion-selective membrane devices. Methods of use of the engineered ionophores is also described.

### BACKGROUND

Metal-ion homeostasis is critical for cellular physiology and requires the efficient and selective transport of metal ions across biological membranes. This process can be aided or disrupted by ionophores, which therefore have diverse applications in basic research, agriculture, and medicine. For instance, Zn(II) ionophores in particular are already widely used as antimicrobial agents. Zn(II) is crucial for the growth and survival of pathogenic bacteria, which have evolved several mechanisms to tightly control their intracellular Zn(II) concentrations, and zinc-binding proteins make up more than 3% of all bacterial proteins. A quinoline-based Zn(II) ionophore, PBT2, was active against gram-positive bacteria and gram-negative bacteria in combination with polymyxin, while another zinc ionophore, pyrithione, is widely used for dandruff and seborrheic dermatitis.

However, despite their enormous promise, contemporary ionophores suffer from several drawbacks, making them a poorly developed class of molecules. Many ionophores operate at high concentrations and are promiscuous, limiting their utility for certain therapeutic applications. Furthermore, several ionophores were discovered serendipitously, possess a complex structure that prevents exhaustive structure-activity relationship studies that is critical for finetuning their activity and specificity. Finally, certain applications (e.g., antimicrobial) require cell-type selectivity (i.e., microbial vs. human), which is low for contemporary ionophores. A general platform for the rational design and rapid development of potent and specific ionophores will significantly enhance the applicability of this promising class of molecules.

A functional ionophore has an optimal binding affinity for its metal ion-a high affinity will prevent ion release in the cell, while a low affinity impairs its carrier function. The ideal ionophore should shield the hydrophilicity of the metal ion to enable membrane permeability and exhibit selectivity for its target ion. Metal-ion chelators with varying affinities, metal specificities, and co-crystal structures are abundant.

Citation or identification of any document in this application is not an admission that such a document is available as prior art to the present invention.

### SUMMARY

In one aspect, the present invention provides for an engineered ionophore comprising: a metal ion chelator group comprising a polar binding site, wherein two or more binding atoms of the polar binding site are capable of binding a metal ion to produce a chelate complex; and one or more shielding group(s) attached to the metal ion chelator group, wherein each shielding group is in proximity to one or more of the binding atom(s), and wherein the chelate complex exhibits increased hydrophobic membrane permeability and reduced metal ion binding affinity as compared to a counterpart chelate complex in the absence of the shielding group(s).

In one example embodiment, the metal ion is selected from zinc (Zn), copper (Cu), iron (Fe), gadolinium (Gd), cobalt (Co), lead (Pb), manganese (Mn), lithium (Li), magnesium (Mg), aluminum (Al), calcium (Ca) and silver (Ag) ions.

In one example embodiment, the metal ion chelator group is selective for a single metal ion.

In one example embodiment, the shielding group(s) independently at each occurrence comprise(s) one or more carbon atom(s), one or more silicon atom(s), one or more germanium atom(s), or any combination thereof.

In one example embodiment, one or more of the shielding group(s) is/are independently at each occurrence selected from alkyl, alkenyl, alkynyl, saturated cyclic hydrocarbon, unsaturated cyclic hydrocarbon, heteroalkyl, heterocyclic ring, aryl ring, and heteroaryl ring group(s), and one or more fused rings thereof, preferably selected from alkyl, heteroalkyl, cycloalkyl, heterocyclic ring, aryl ring, and heteroaryl ring group(s), more preferably selected from C₄ or greater alkyl group(s).

In one example embodiment, one or more of the shielding group(s) is/are independently at each occurrence selected from organosilyl group(s), preferably selected from trialkyl organosilyl groups, alkyldiaryl organosilyl groups, dialkylaryl organosilyl groups, and triaryl organosilyl groups, more preferably, wherein each alkyl group is independently at each occurrence selected from C₁-C₆ alkyl groups.

In one example embodiment, one or more of the shielding group(s) is/are independently at each occurrence selected from organogermanyl group(s), preferably selected from trialkyl or triphenyl organogermanyl groups, more preferably, wherein each alkyl group is independently at each occurrence selected from C₁-C₆ alkyl groups.

In one example embodiment, one or more of the shielding group(s) is/are independently at each occurrence in an ortho-position to one of the binding atom(s).

In one example embodiment, the metal ion chelator group and/or the metal ion is/are selected from Table 1 of the present invention, wherein the shielding group(s) is/are independently at each occurrence selected from Table 2 of the present invention, and/or wherein the metal ion chelator group and/or the shielding group(s) is/are independently at each occurrence selected from Table 3 of the present invention.

In one aspect, the present invention provides for a pharmaceutical composition comprising at least one engineered ionophore of the present invention, and, optionally, at least metal ion capable of binding with the at least one engineered ionophore.

In one aspect, the present invention provides for a kit comprising at least one engineered ionophore of the present invention, and, optionally, at least one metal ion capable of binding with the at least one engineered ionophore.

In one aspect, the present invention provides for an ion-selective membrane device comprising at least one hydrophobic membrane, at least one engineered ionophore of the present invention, and at least one metal ion capable of binding with the at least one engineered ionophore.

In one example embodiment, the ion-selective membrane device is selected from ion-selective membrane electrodes or ion-selective membrane sensors.

In one aspect, the present invention provides for a method of increasing hydrophobic membrane transport of metal ions, the method comprising: forming a reaction mixture comprising: a metal ion; and an engineered ionophore of the present invention, wherein a chelate complex is formed; and contacting the reaction mixture with a hydrophobic membrane, wherein the chelate complex is transported across the hydrophobic membrane and the metal ion is released from the transported chelate complex, and wherein the chelate complex expresses increased hydrophobic membrane transport and/or metal ion release as compared to a counterpart chelate complex in the absence of the shielding group(s).

In one example embodiment, the hydrophobic membrane is a cellular membrane, and wherein: when the reaction mixture is formed and contacted with the cellular membrane outside of a cell, a vesicle, or a cellular organelle, the chelate complex is transported across the cellular membrane into the cell, the vesicle, or the cellular organelle; and when the reaction mixture is formed and contacted with the cellular membrane inside of a cell, a vesicle, or a cellular organelle, the chelate complex is transported across the cellular membrane and out of the cell, the vesicle, or the cellular organelle.

In one example embodiment, the contacting the reaction mixture with a cellular membrane occurs in a human or part thereof, or a non-human animal, a plant, or a part thereof, and wherein the method targets, diagnoses, treats, prevents, or any combination thereof, a current or potential disease, disease state, condition, disorder, side effect, or the like, or any combination thereof, of the human or the part thereof, or the non-human animal, the plant, or the part thereof. In one example embodiment, the current or potential disease, disease state, condition, disorder, side effect, or the like, or any combination thereof, is selected from a metal ion deficiency, a metal ion overload, a microbial infection, cancer, chronic kidney disease, Alzheimer's disease, ALS, Wilson disease, diabetes, and sickle cell anemia. In one example embodiment, the current or potential disease, disease state, condition, disorder, side effect, or the like, or any combination thereof, is a microbial infection, and wherein the method increases antimicrobial activity against the infection in the human or the part thereof, or the non-human animal, the plant, or the part thereof.

In one example embodiment, the method further comprises, prior to, concurrently with, or after forming the reaction mixture, administering the pharmaceutical composition of the present invention to the human or part thereof, or the non-human animal, the plant, or the part thereof.

In one example embodiment, the method occurs within the ion-selective membrane device of the present invention.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1F—(FIG. 1A) Chemical structure of zinc ionophores. (FIG. 1B) Relative fluorescence intensity of U2OS cells (n=3), wherein 10 μM of Zn(II) and 10 μM of compound were co-incubated for 1 hour, followed by the addition of DA-ZP1. (FIG. 1C) Representative images of DA-ZP1-treated U2OS cells under the FITC channel (top) measuring ZP1 release and DAPI staining (bottom). Scale bar=100 μm. (FIG. 1D) Relative fluorescent intensity of U2OS cells (n=3), wherein 10 μM of Zn(II) and 5 μM of compound were co-incubated for 1 hour, followed by the addition of DA-ZP1. (FIG. 1E) Representative images of DA-ZP1-treated U2OS cells under the FITC channel (top) measuring ZP1 release and DAPI staining (bottom). Scale bar =100 μm. (FIG. 1F) ICP-MS analysis of 1, 8, and 9, wherein 10 μM of Zn(II) and 5 μM of compound were co-incubated for 1 hour (n=5).

FIG. 2—Representative images of DA-ZP1-treated U2OS cells under the FITC channel (top) measuring ZP1 release and DAPI staining (bottom), wherein 10 μM of Zn(II and 10 μM of each compound were co-incubated for 1 hour, followed by the addition of DA-ZP1. ZI1: tetrabutylthiuram disulfide. ZI4: potassium hydrotris(N-tert-butyl-2-thioimidazolyl) borate. Pyr: pyrithione. CHQ: chloroquine. HQ: 8-hydroxy-quinoline. CLQ: clioquinol. Scale bar=100 μm.

FIG. 3A-3D—(FIG. 3A, 3B) X-ray structure of compound 1 (FIG. 3A) and compound 9 (FIG. 3B). (FIG. 3C) Kinetic study of DA-ZP1 fluorescence turn-on with co-incubation of 10 μM of various compounds and 10 μM of ZnII. (FIG. 3D) ¹H NMR of Zn: 1 and Zn: 9.

FIG. 4A-4B—(FIG. 4A) Relative fluorescence intensity of INS-1E cells (n=3), wherein DA-ZP1 and 20 μM of each compound were co-incubated for 1 hour. Pyr: pyrithione. (FIG. 4B) Representative images of DA-ZP1-treated INS-1E cells under the FITC channel (top) measuring ZP1 release and DAPI staining (bottom). Scale bar=100 μm.

FIG. 5A-5D—(FIG. 5A) The minimum inhibitory concentration (MIC) was determined in the absence (untreated) or presence of compounds in indicated concentrations. Cell survival is reflected in OD600 where a value <0.1 is indicated in white for determining the MIC. MIC analysis of zinc ionophores and natural polyether ionophores (n=3) in Staphylococcus epidermidis, Staphylococcus caprae, Bacillus subtilis, and Bacillus cereus. (FIG. 5B) MIC analysis of Staphylococcus epidermidis in the presence of EDTA with 9 (n=3). (FIG. 5C) MIC analysis of Staphylococcus epidermidis with the addition of Zn(II) with 9 (n=3). (FIG. 5D) U2OS cell viability assay with co-incubations in the presence of 5 μM of Zn(II) (n=3).

FIG. 6—Overview of the ionophore activity and antibacterial activity and selectivity of Zn(II) ionophores.

FIG. 7—Space filling model of compound 1 chelated to Zn(II).

FIG. 8A-8B—(FIG. 8A) Representative images of DA-ZP1-treated U2OS cells under the FITC channel (top) measuring ZP1 release, DAPI staining (middle), and merge (bottom). Before adding DA-ZP1, 10 μM of Zn(II) and 10 μM of compound were added. Scale bar=100 μm. (FIG. 8B) Representative images of DA-ZP1-treated U2OS cells under the FITC channel (top) measuring ZP1 release, DAPI staining (middle), and merge (bottom). Before adding DA-ZP1, 10 μM of Zn(II), and 5 μM of compound were added. Scale bar=100 μm.

FIG. 9—Representative images of FluoZin-3-treated U2OS cells under the FITC channel (top), DAPI staining (middle), and merge (bottom). Before adding DA-ZP1, 10 μM of Zn(II) and compound 8 at the indicated concentration were added. Scale bar=100 μm.

FIG. 10A-10B—(FIG. 10A) A) Representative images of DA-ZP1-treated U2OS cells under the FITC channel (top) measuring ZP1 release, DAPI staining (middle), and merge (bottom). Before adding DA-ZP1, 10 μM of Zn(II), 10 μM of indicated metal ion, and 10 μM of compound 8 were added. Scale bar=100 μm. (FIG. 10B) Relative fluorescence intensity of U2OS cells (n=3), metal ions (10 μM) were co-incubated with 10 μM of Zn(II) and 10 μM of 8 (n=3).

FIG. 11A-11B—(FIG. 11A) Representative images of DA-ZP1-treated U2OS cells under the FITC channel (top) measuring ZP1 release, DAPI staining (middle), and merge (bottom). Before adding DA-ZP1, 10 μM of Zn (11) and 10 μM of compound were added. Scale bar=100 μm. (FIG. 11B) Representative images of DA-ZP1-treated U2OS cells under the FITC channel (top) measuring ZP1 release, DAPI staining (middle), and merge (bottom). Before adding DA-ZP1, 10 μM of Zn (11) and 5 μM of compound were added. Scale bar=100 μm. ZI1: tetrabutylthiuram disulfide. ZI4: potassium hydrotris(N-tert-butyl-2-thioimidazolyl) borate. Pyr: pyrithione. CHQ: chloroquine. HQ: 8-hydroxy-quinoline. CLQ: clioquinol.

FIG. 12—UV/Vis spectra of compounds at different ZnCl₂ concentrations measured in acetonitrile/H₂O. Absorption at the indicated 2 of compounds at different ZnCl₂ concentrations. Stability constants for compounds were determined by UV/Vis titration using the TitrationFit software.

FIG. 13—(FIG. 13A) Perspective views showing 50% probability displacement. (FIG. 13B) Three-dimensional supramolecular architecture viewed along the α-axis direction. (FIG. 13C) Superimposed X-ray structure of Zn:1 and Zn:9.

FIG. 14A-14B—(FIG. 14A) Representative images of DA-ZP1-treated INS-1E cells under the FITC channel (top) measuring ZP1 release, DAPI staining (middle), and merge (bottom). Before adding DA-ZP1, 20 μM of compound was added. Scale bar 100 μm. (FIG. 14B) Representative images of DA-ZP1-treated INS-1E cells under the FITC channel (top) measuring ZP1 release, DAPI staining (middle), and merge (bottom). Before adding DA-ZP1, 10 μM of compound was added. Scale bar=100 μm. Pyr: pyrithione.

FIG. 15—The minimum inhibitory concentration (MIC) was determined in the absence (untreated) or presence of compounds in indicated concentrations. Cell survival is reflected in OD₆₀₀ where a value <0.1 is indicated in white for determining the MIC. MIC analysis of zinc ionophores and natural polyether ionophores (n=3) in Staphylococcus epidermidis.

FIG. 16—Cell viability assay with co-incubations in the absence (blue) or presence (red) of 5 μM of Zn(II) (red) (n=3). (FIG. 16A) Compound 1. (FIG. 16B) Compound 4. (FIG. 16C) Compound 6. (FIG. 16D) Compound 8. (FIG. 16E) Compound 9. (FIG. 16F) Pyrithione.

FIG. 17—A high-throughput assay to measure ionophore activity in cells using DA-ZP1.

FIG. 18—Proximally located bulky/hydrophobic substituents increased ionophore activity.

FIG. 19—Proximity of bulky/hydrophobic substituent to Zn(II) binding pocket is critical.

FIG. 20—Design principle for converting chelator to ionophore.

FIG. 21—Silicon offers several advantages over carbon for ionophore development.

FIG. 22—Modular synthesis of silicon containing ionophores.

FIG. 23—Silyl-substitution allowed development of ionophore with graded activity.

FIG. 24—Orthogonal measurement of ionophore activity with Inductively Coupled Plasma Mass Spectrometry (ICP-MS).

FIG. 25—Silyl-based ionophores selectively bind to Zn(II) over other metals.

FIG. 26—Silyl-based ionophores outperform known zinc ionophores.

FIG. 27—Validation of design principles. Silyl groups lower binding affinity to Zn(II).

FIG. 28—X-ray structure suggests shielding of ionophore's polar surface by silyl group.

FIG. 29—Silyl-based ionophore easily exchanges Zn(II) with DA-ZP1 while chelators don't.

FIG. 30—NMR peak broadening confirms fluxionality with ionophore: Zn(II) interaction.

FIG. 31—Silyl-based ionophore exhibit potent antibacterial activity.

FIG. 32—Ionophore L11 exhibited the best antibacterial activity of all Silyl-based ionophores.

FIG. 33—Sequestration of Zn(II) using EDTA reduced ionophore activity.

FIG. 34—Silyl-based ionophore possesses higher antibacterial selectivity over pyrithione.

FIG. 35—Silyl-based ionophores were more selective than pyrithione in other strains.

FIG. 36—Ionophore activity, enhanced antibacterial activity and selectivity, and generality of molecular design.

FIG. 37—Ionophores enhance fluorescent intensity of DA-ZP1 in β cells.

FIG. 38—Ionophores elevate Zn(II) concentration of cytoplasm in β cells.

FIG. 39—Development of copper ionophore-generalization of molecular design.

FIG. 40—Studies towards conversion of copper chelators to ionophores.

FIGS. 41-72—¹H NMR spectra and ¹³C NMR spectra of ionophores.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this invention pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this invention, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide engineered ionophores capable of increased transport of metal ions across hydrophobic membranes and/or reduced binding affinity with said metal ions. In one aspect, the present invention provides for an engineered ionophore comprising: a metal ion chelator group comprising a polar binding site, wherein one or more binding atom(s) of the polar binding site are capable of binding a metal ion to produce a chelate complex; and one or more shielding group(s) attached to the metal ion chelator group, wherein each shielding group is in proximity to one or more of the binding atom(s), and wherein the chelate complex exhibits increased hydrophobic membrane permeability and reduced metal ion binding affinity as compared to a counterpart chelate complex in the absence of the shielding group(s).

Embodiments disclosed herein further provide methods of increasing transport of metal ions across hydrophobic membranes, said methods comprising contacting said membranes with said engineered ionophores, metal ion chelate complexes thereof, pharmaceutical compositions thereof, the components of kits thereof, or the like, or any combination thereof. In example embodiments, said hydrophobic membranes are biological membranes, such as for example, cell membranes. In example embodiments, said methods comprise administering said engineered ionophores, metal ion chelate complexes thereof, pharmaceutical compositions thereof, the components of kits thereof, or the like, or any combination thereof, to a human or a non-human animal, a plant, or a part thereof. In various example embodiments, said methods target, diagnose, treat, prevent, or any combination thereof, a current or potential disease, disease state, condition, disorder, side effect, or the like, or any combination thereof. In example embodiments, said hydrophobic membranes are ion-selective membranes. In example embodiments, said methods occur within ion-selective membrane devices of the present invention.

### Ionophores and Chelate Complexes Thereof

As used herein, unless otherwise specified, the term “chelating agent” or “chelating group” or “chelator” refers to a compound or group which binds a metal ion by two or more points of contact, e.g. via two separate coordinate bonds at binding atoms of the compound or group, to form a chelate complex.

As used herein, unless otherwise specified, the term “ionophore” refers to a subset of chelating agents. Ionophores are generally more lipophilic than chelators. Ionophore chelate-complexes are known to enable transport of polar metal ions across hydrophobic membranes, e.g., cell membranes, such as, for example, into cells and/or out of cells, or ion-selective membranes, such as for examples, for ion-selective membrane electrodes.

Ionophores are known in the art (see Roy and Talukdar, “Recent Advances in Bioactive Artificial Ionophores”, ChemBioChem 2021, 22, 2925; Steinbruek et al., “Transition metal chelators, pro-chelators, and ionophores as small molecule cancer chemotherapeutic agents”, Chem. Soc. Rev. 2020, 49, 3726; Oliveri, “Biomedical applications of copper ionophores”, Coord. Chem. Rev. 2020, 422, 213474; Müller et al., “CD44 regulates epigenetic plasticity by mediating iron endocytosis”, Nat. Chem. 2020, 12, 929; Liu et al., “Chemical Syntheses and Chemical Biology of Carboxyl Polyether Ionophores: Recent Highlights”, Angew. Chem. Int. Ed. 2019, 58, 13630; Vaden et al., “A Cancer-Selective Zinc Ionophore Inspired by the Natural Product Naamidine A”, ACS Chem. Biol. 2019, 14, 106; Helsel and Franz, “Pharmacological activity of metal binding agents that alter copper bioavailability.” Dalton Transactions 2015, 44.19, 8760; Tardito et al., “Copper-Dependent Cytotoxicity of 8-Hydroxyquinoline Derivatives Correlates with Their Hydrophobicity and Does Not Require Caspase Activation”, J. Med. Chem. 2012, 55, 10448; Tardito et al., “Copper Binding Agents Acting as Copper Ionophores Lead to Caspase Inhibition and Paraptotic Cell Death in Human Cancer Cells”, Am. Chem. Soc. 2011, 133, 16, 6235; Kevin I I et al., Polyether ionophores: broad-spectrum and promising biologically active molecules for the control of drug-resistant bacteria and parasites, Expert Opin. Drug Discov. 2009, 4:2, 109; and Magda et al., “Synthesis and anticancer properties of water-soluble zinc ionophores”, Cancer Res. 2008, 68 (13), 5318).

### Engineered Ionophores and Metal Ion Chelate Complexes Thereof

In one aspect, the present invention provides an engineered ionophore capable of forming a chelate complex with a metal ion (e.g., an ionophore chelate complex). In one example embodiment, the engineered ionophore comprises: a metal ion chelator group comprising a polar binding site, wherein one or more binding atom(s) of the polar binding site are capable of binding a metal ion to produce a chelate complex; and one or more shielding group(s) attached to the metal ion chelator group, wherein each shielding group is in proximity to one or more of the binding atom(s).

In one example embodiment, the engineered ionophore is capable of forming a chelate complex exhibiting increased hydrophobic membrane permeability (e.g., permeability of a biological membrane (e.g., a cell membrane or the like) or a nonbiological membrane (e.g., a glass, a crystalline, a polymer membrane, or the like) as compared to a counterpart chelate complex in the absence of the shielding group(s). In one example embodiment, the engineered ionophore is capable of forming a chelate complex exhibiting reduced metal ion binding affinity as compared to a counterpart chelate complex in the absence of the shielding group(s).

### Metal Ions

In one example embodiment, a metal ion is chosen from alkali metals, alkaline earth metals, transition metals, post-transition (or basic) metals, metalloids (or semi-metals), lanthanides, and actinides. In one example embodiment, a metal ion has any possible charge (which may also be referred to herein as a valence or an oxidation state) of the metal. In one example embodiment, a metal ion is chosen from zinc (Zn), copper (Cu), iron (Fe), cobalt (Co), manganese (Mn) lead (Pb), gadolinium (Gd), lithium (Li), magnesium (Mg), aluminum (Al), calcium (Ca) and silver (Ag) ions.

### Metal Ion Chelators

In one example embodiment, an engineered ionophore has an affinity for a group of metal ions or a selectivity for a single metal ion. Suitable metal ion chelators are known in the art (see Steinbrueck et al., “Transition metal chelators, pro-chelators, and ionophores as small molecule cancer chemotherapeutic agents”, Chem. Soc. Rev. 2020, 49, 3726; Mautner et al., “Five-Coordinated Geometries from Molecular Structures to Solution in Copper (II) Complexes Generated from Polydentate-N-donor Ligands and Pseudohalides”, Molecules 2020, 25, 3376; Rakshit et al., “Cu²⁺ selective chelators relieve copper-induced oxidative stress in vivo”, Chem. Sci. 2018, 9, 7916; Vanasschen et al., “Novel CDTA-based, Bifunctional Chelators for Stable and Inert Mn^{II} Complexation: Synthesis and Physicochemical Characterization”, Inorg. Chem. 2017, 56, 7746; Hancock, “The pyridyl group in ligand design for selective metal ion complexation and sensing”, Chem. Soc. Rev. 2013, 42, 1500; Que et al., “Metals in Neurobiology: Probing Their Chemistry and Biology with Molecular Imaging”, Chem. Rev. 2008, 108, 1517; and Haas et al., “Application of Metal Coordination Chemistry To Explore and Manipulate Cell Biology”, Chem. Rev. 2009, 109, 4921).

Non-limiting examples of suitable metal ion chelator groups are shown in Table 1. Unless indicated as having selectivity for an indicted ion/ions, a metal ion chelator group shown in Table 1 has an affinity for binding with the indicated ion/ions but is not limited to (e.g., selective for) binding with said ion/ions. In one example embodiment, a metal ion and/or metal ion chelator group is/are chosen from Table 1.

TABLE 1

| Metal Ion | Metal Ion Chelator Group Structures |
|---|---|
| Affinity for Zn²⁺ | 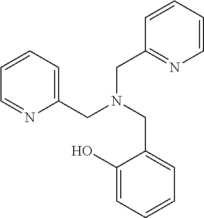 [SMILES: Oc1ccccc1CN(Cc1ccccn1)Cc1ccccn1] |
| | 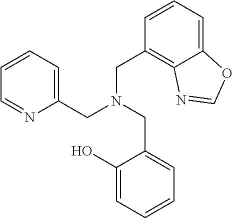 [SMILES: Oc1ccccc1CN(Cc1ccccn1)Cc1cccc2ocnc12] |
| | 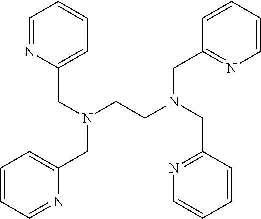 [SMILES: c1ccc(CN(CCN(Cc2ccccn2)Cc2ccccn2)Cc2ccccn2)nc1] |
| | 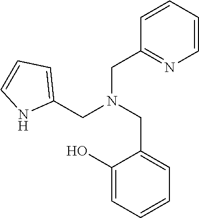 [SMILES: Oc1ccccc1CN(Cc1ccccn1)Cc1ccc[nH]1] |
| | 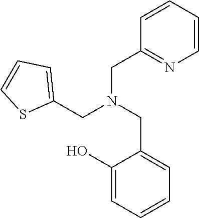 [SMILES: Oc1ccccc1CN(Cc1ccccn1)Cc1cccs1] |
| | 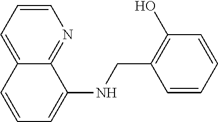 [SMILES: Oc1ccccc1CNc1cccc2cccnc12] |
| | 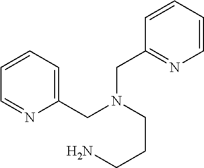 [SMILES: NCCCN(Cc1ccccn1)Cc1ccccn1] |
| | 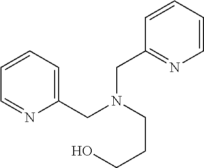 [SMILES: OCCCN(Cc1ccccn1)Cc1ccccn1] |
| | 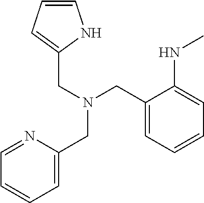 [SMILES: CNc1ccccc1CN(Cc1ccccn1)Cc1ccc[nH]1] |
| | 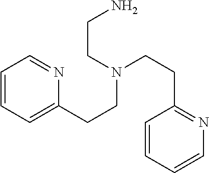 [SMILES: NCCN(CCc1ccccn1)CCc1ccccn1] |
| | 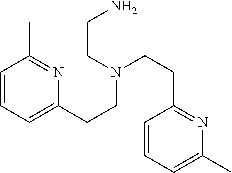 [SMILES: Cc1cccc(CCN(CCN)CCc2cccc(C)n2)n1] |
| | 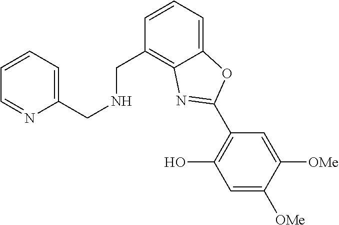 [SMILES: COc1cc(O)c(-c2nc3c(CNCc4ccccn4)cccc3o2)cc1OC] |
| | 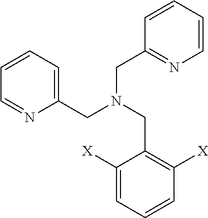 |
| | X = OH, NH₂ |
| | 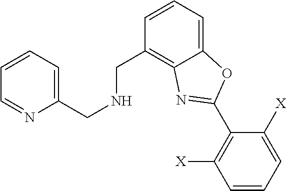 |
| | X = OH, NH₂ |
| | 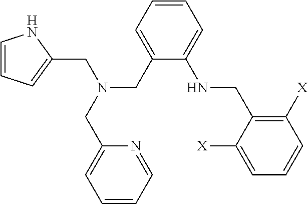 |
| | X = OH, NH₂ |
| | 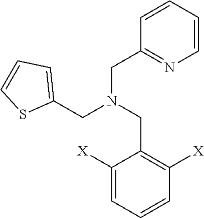 |
| | X = OH, NH₂ |
| | 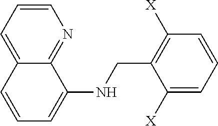 |
| | X = OH, NH₂ |
| | 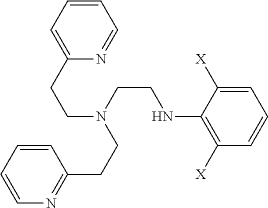 |
| | X = OH, NH₂ |
| | 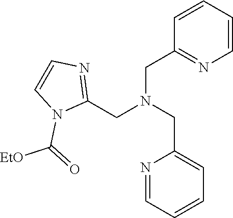 [SMILES: CCOC(=O)n1ccnc1CN(Cc1ccccn1)Cc1ccccn1] |
| Affinity for Cu²⁺ | 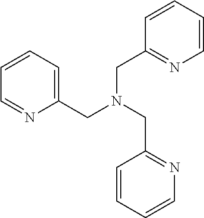 [SMILES: c1ccc(CN(Cc2ccccn2)Cc2ccccn2)nc1] |
| | 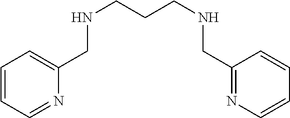 [SMILES: c1ccc(CNCCCNCc2ccccn2)nc1] |
| | 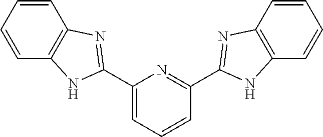 [SMILES: c1cc(-c2nc3ccccc3[nH]2)nc(-c2nc3ccccc3[nH]2)c1] |
| | 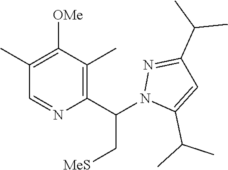 [SMILES: COc1c(C)cnc(C(CSC)n2nc(C(C)C)cc2C(C)C)c1C] |
| | 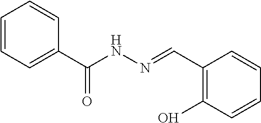 [SMILES: O=C(N/N=C/c1ccccc1O)c1ccccc1] |
| | 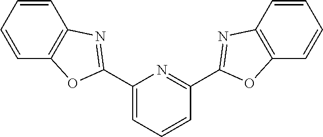 [SMILES: c1cc(-c2nc3ccccc3o2)nc(-c2nc3ccccc3o2)c1] |
| | 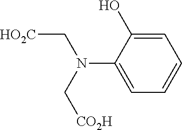 [SMILES: O=C(O)CN(CC(=O)O)c1ccccc1O] |
| | 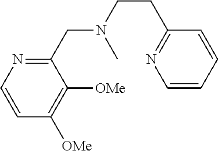 [SMILES: COc1ccnc(CN(C)CCc2ccccn2)c1OC] |
| | 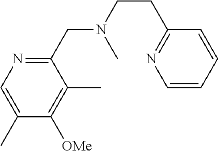 [SMILES: COc1c(C)cnc(CN(C)CCc2ccccn2)c1C] |
| | 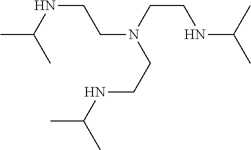 [SMILES: CC(C)NCCN(CCNC(C)C)CCNC(C)C] |
| | 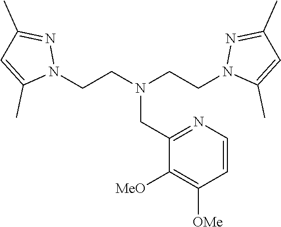 [SMILES: COc1ccnc(CN(CCn2nc(C)cc2C)CCn2nc(C)cc2C)c1OC] |
| | 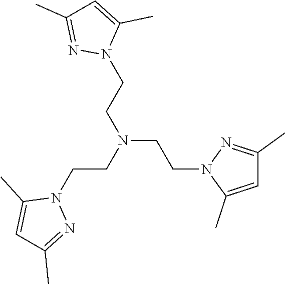 |
| | 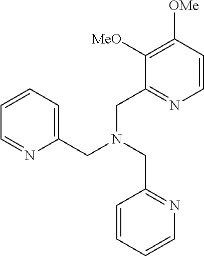 [SMILES: COc1ccnc(CN(Cc2ccccn2)Cc2ccccn2)c1OC] |
| Selective for Cu²⁺ | 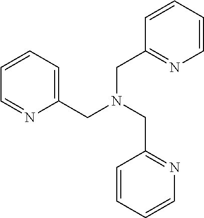 [SMILES: c1ccc(CN(Cc2ccccn2)Cc2ccccn2)nc1] |
| | 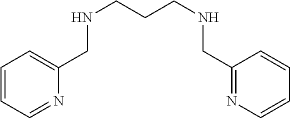 [SMILES: c1ccc(CNCCCNCc2ccccn2)nc1] |
| | 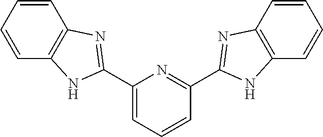 [SMILES: c1cc(-c2nc3ccccc3[nH]2)nc(-c2nc3ccccc3[nH]2)c1] |
| | 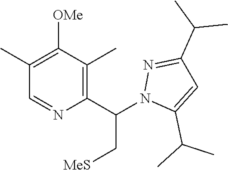 [SMILES: COc1c(C)cnc(C(CSC)n2nc(C(C)C)cc2C(C)C)c1C] |
| | 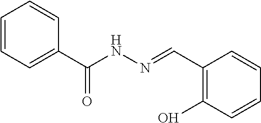 [SMILES: O=C(N/N=C/c1ccccc1O)c1ccccc1] |
| Affinity for Fe^{2+/3+} | 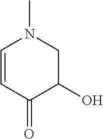 |
| | 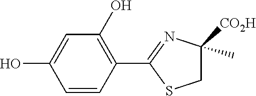 |
| | 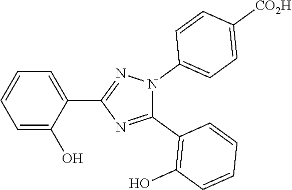 [SMILES: O=C(O)c1ccc(-n2nc(-c3ccccc3O)nc2-c2ccccc2O)cc1] |
| | 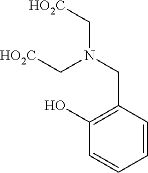 [SMILES: O=C(O)CN(CC(=O)O)Cc1ccccc1O] |
| | 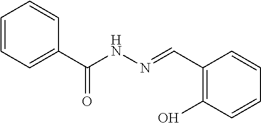 [SMILES: O=C(N/N=C/c1ccccc1O)c1ccccc1] |
| | 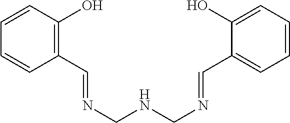 [SMILES: Oc1ccccc1/C=N/CNC/N=C/c1ccccc1O] |
| | 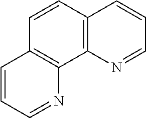 [SMILES: c1cnc2c(c1)ccc1cccnc12] |
| | 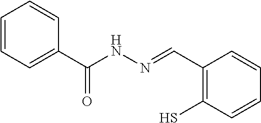 [SMILES: O=C(N/N=C/c1ccccc1S)c1ccccc1] |
| | 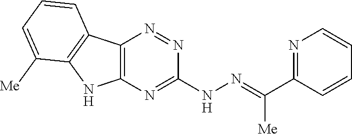 [SMILES: C/C(=N\Nc1nnc2c(n1)[nH]c1c(C)cccc12)c1ccccn1] |
| | 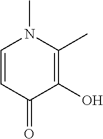 |
| | 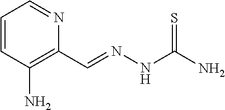 [SMILES: NC(=S)N/N=C/c1ncccc1N] |
| | 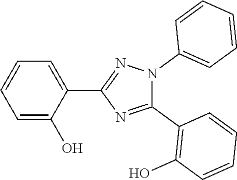 [SMILES: Oc1ccccc1-c1nc(-c2ccccc2O)n(-c2ccccc2)n1] |
| | 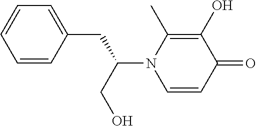 [SMILES: Cc1c(O)c(=O)ccn1[C@H](CO)Cc1ccccc1] |
| Selective for Fe^{2+/3+} | 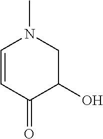 |
| | 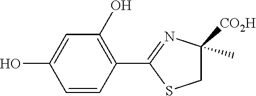 |
| | 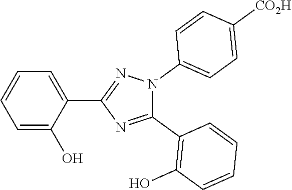 [SMILES: O=C(O)c1ccc(-n2nc(-c3ccccc3O)nc2-c2ccccc2O)cc1] |
| | 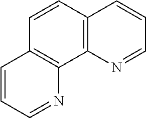 [SMILES: c1cnc2c(c1)ccc1cccnc12] |
| | 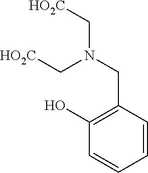 [SMILES: O=C(O)CN(CC(=O)O)Cc1ccccc1O] |
| | 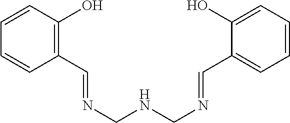 [SMILES: Oc1ccccc1/C=N/CNC/N=C/c1ccccc1O] |
| | 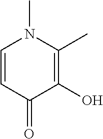 |
| | 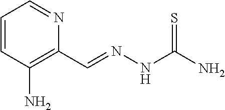 [SMILES: NC(=S)N/N=C/c1ncccc1N] |
| | 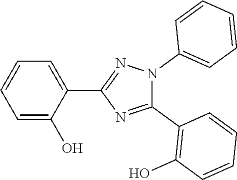 [SMILES: Oc1ccccc1-c1nc(-c2ccccc2O)n(-c2ccccc2)n1] |
| | 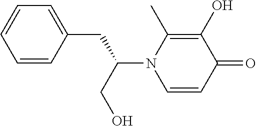 [SMILES: Cc1c(O)c(=O)ccn1[C@H](CO)Cc1ccccc1] |
| Affinity for Gd³⁺ | 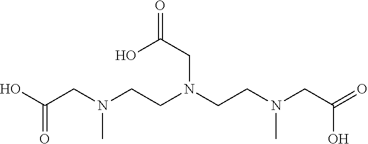 [SMILES: CN(CCN(CCN(C)CC(=O)O)CC(=O)O)CC(=O)O] |
| | 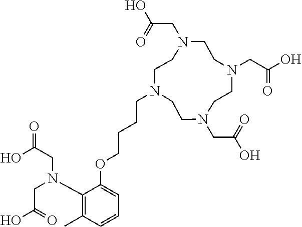 [SMILES: Cc1cccc(OCCCCN2CCN(CC(=O)O)CCN(CC(=O)O)CCN(CC(=O)O)CC2)c1N(CC(=O)O)CC(=O)O] |
| Affinity for Co²⁺ | 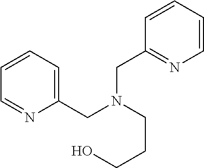 [SMILES: OCCCN(Cc1ccccn1)Cc1ccccn1] |
| Affinity for Mn²⁻ | 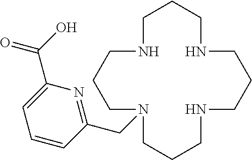 [SMILES: O=C(O)c1cccc(CN2CCCNCCCNCCCNCCC2)n1] |

### Shielding Groups

In one example embodiment, a shielding group independently at each occurrence comprises one or more carbon atoms, one or more silicon atoms, one or more germanium atoms, or any combination thereof. In one example embodiment, a shielding group is independently at each occurrence selected from alkyl, alkenyl, alkynyl, saturated cyclic hydrocarbon, unsaturated cyclic hydrocarbon, heteroalkyl, heterocyclic ring, aryl ring, and heteroaryl ring groups, and one or more fused rings thereof, preferably selected from alkyl, heteroalkyl, cycloalkyl, heterocyclic ring, aryl ring, and heteroaryl ring groups, more preferably selected from C₄ or greater alkyl groups.

In one example embodiment, a shielding group is independently at each occurrence selected from organosilyl groups, preferably selected from trialkyl organosilyl groups, alkyldiaryl organosilyl groups, dialkylaryl organosilyl groups, and triaryl organosilyl groups, more preferably, wherein each alkyl group is independently at each occurrence selected from C₁-C₄ alkyl groups.

In one example embodiment, one or more of the shielding group(s) is/are independently at each occurrence selected from organogermanyl group(s), preferably selected from trialkyl or triphenyl organogermanyl groups, more preferably, wherein each alkyl group is independently at each occurrence selected from C₁-C₆ alkyl groups.

Non-limiting examples of suitable shielding groups are shown in Table 2. In one example embodiment, a shielding group is independently at each occurrence chosen from Table 2.

TABLE 2
Shielding Groups
Organic Groups

| | |
|---|---|
| Alkyl: —R | R = ⁱPr, ^{t}Bu, ⁱBu, ⁿBu, sec-Bu |

Organo-Silane Groups

| | |
|---|---|
| Silyl: —SiR¹R²R³ | R¹ = R² = R³ = Me, Et, ⁱPr, ^{t}Hx, or Ph |
| | R¹ = R² = Me, R³ = Et, ⁱPr, ^{t}Bu, Et, |
| | ⁱPr, ^{t}Hx, or Ph |
| | R¹ = R² = Et, R³ = Me, ⁱPr, ^{t}Bu, Et, |
| | ⁱPr, ^{t}Hx, or Ph |
| | R¹ = R² = Ph, R³ = Me, Et, ⁱPr, ^{t}Bu, |
| | Et, ⁱPr, or ^{t}Hx |
| Cyclic Silyl: | R¹ = Me, Et, ⁱPr, ^{t}Hx, or Ph |
| 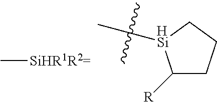 | |

Organo-Germanium Groups

| | |
|---|---|
| Germanyl: —GeR¹R²R³ | R¹ = R² = R³ = Me, Et or Ph |

In one example embodiment, a shielding group is independently at each occurrence in an ortho-position to one of the binding atoms.

Non-limiting examples of suitable combinations of metal ion chelator groups and shielding groups are shown in Table 3. In one example embodiment, a metal ion chelator group and/or a shielding group is independently at each occurrence chosen from Table 3.

TABLE 3

| | | | |
|---|---|---|---|
| 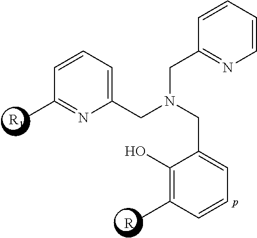 | R = H, R₁ = H R = Me, R₁ = H R = ⁱPr, R₁ = H R = ^{t}Bu, R₁ = H R = SiR₁, R₁ = H R′ = (Me)₃, (Et)₃, (ⁱPr)₃, Me₂^{t}Bu, Me₂Ph, Ph₂^{t}Bu, Ph₂Me R = R₁ = Me, ⁱPr, ^{t}Bu R = R₁ = SiR′ R′ = (Me)₃, (Et)₃, (ⁱPr)₃, Me₂^{t}Bu, Me₂Ph, Ph₂^{t}Bu, Ph₂Me | | |
| 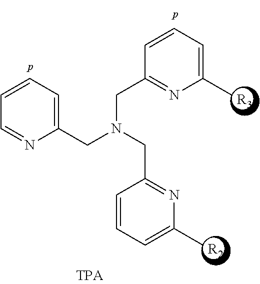 | 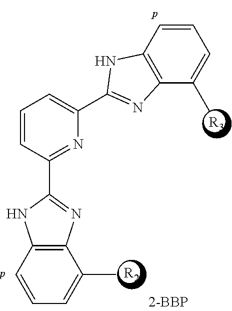 | 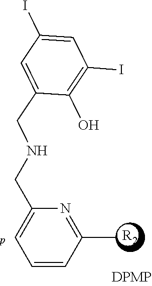 | R₂ = H, R₃ = Me, ⁱPr, ^{t}Bu R₂ = R₃ = Me, ⁱPr, ^{t}Bu R₂ = R₃ = ^{t}Bu(p) R₂ = H, R₃ = SiR′ R₂ = R₃ = SiR′ R′ = (Me)₃, (Et)₃, (ⁱPr)₃, Me₂^{t}Bu, Me₂Ph, Ph₂^{t}Bu, Ph₂Me |
| 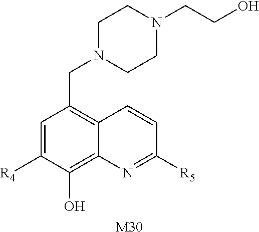 | 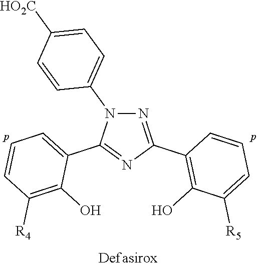 | R₄ = H, R₅ = ^{t}Bu R₄ = R₅ = ^{t}Bu R₄ = R₅ = ^{t}Bu(p) R₄ = H, R₅ = SiR′ R₄ = R₅ = SiR′ R′ = (Me)₃, (Et)₃, (ⁱPr)₃, Me₂^{t}Bu, Me₂Ph, Ph₂^{t}Bu, Ph₂Me | |

### Pharmaceutical Compositions of Engineered Ionophores

In one aspect, the present invention provides for pharmaceutical composition comprising at least one engineered ionophore of the present invention, and, optionally, at least metal ion capable of binding with the at least one engineered ionophore. In one example embodiment, no metal ion is chelated with an engineered ionophore. In one example embodiment, one or more or all metal ions is/are in the form of an ionophore metal ion chelate complex with an engineered ionophore.

### Pharmaceutical Formulation

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient, a CRISPR-Cas system or component thereof described in greater detail elsewhere herein. In some embodiments, the pharmaceutical formulation can include, such as an active ingredient, a CRISPR-Cas polynucleotide described in greater detail elsewhere herein. In some embodiments, the pharmaceutical formulation can include, such as anactive ingredient one or more modified cells, such as one or more modified cells described in greater detail elsewhere herein.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a hematopoietic disease or a symptom thereof. In some embodiments, the subject in need thereof has or is suspected of having, a neurobiological disease or disorder, a psychiatric disease or disorder, a cancer, an autoimmune disease or disorder, a thrombosis disease, a heart disease, a kidney disease, a lung disease, or a blood vessel disease, or a combination thereof.

As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological orphysiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects.

The effective amount, least effective amount, and/or therapeutically effective amount of the primary and optional secondary active agent described elsewhere herein contained in the pharmaceutical formulation can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges.

The therapeutically effective amount can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges.

In other embodiments, the effective amount, least effective amount, and/or therapeutically effective amount of the primary and optional secondary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent present in the pharmaceutical formulation can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49,

0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments where a cell population is present in the pharmaceutical formulation (e.g., as a primary and/or or secondary active agent), the effective amount of cells can range from about 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL.

In some embodiments, the amount or effective amount, particularly where an infective particle is being delivered (e.g. a virus particle having the primary or secondary agent as a cargo), the effective amount of virus particles can be expressed as a titer (plaque forming units per unit of volume) or as a MOI (multiplicity of infection). In some embodiments, the effective amount can be 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wlkins, Baltimore, MD, 2000, and “Pharmaceutical dosage forms and drug delivery systems”, 6th Edition, Ansel et al., (Media, PA: Wlliams and Wlkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof. Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g. micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, manitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein can be part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatoirenti-histamines, anti-infectives, chemotherapeutics, and combinations thereof.

### Administration of Pharmaceutical Formulations

The pharmaceutical formulations or dosage forms thereof described herein can be administered one or more times hourly, daily, monthly, or yearly (e.g. 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g. 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g. within seconds or just a few minutes apart), where the intent is that the formulations be administered together at the same time.

In embodiments, the treatment is for disease/disorder of an organ, including liver disease, eye disease, muscle disease, heart disease, blood disease, brain disease, kidney disease, or may comprise treatment for an autoimmune disease, central nervous system disease, cancer and other proliferative diseases, neurodegenerative disorders, inflammatory disease, metabolic disorder, musculoskeletal disorder and the like.

Methods for modifying a programmable nuclease of interest are also provided, the method comprising contacting the programmable nuclease of interest with a molecule or a composition disclosed herein. Methods for the treatment of a disease, disorder, or condition in a subject in need thereof can comprise administering a molecule or a composition disclosed herein to a subject.

### Kits of Engineered Ionophores

In one aspect, the present invention provides for a kit comprising at least one engineered ionophore of the present invention, and, optionally, at least metal ion capable of binding with the at least one engineered ionophore. In one example embodiment, no metal ion is chelated with an engineered ionophore. In one example embodiment, one or more or all metal ions is/are in the form of an ionophore metal ion chelate complex with an engineered ionophore.

### Ion-Selective Membrane Devices of Engineered Ionophores

In one aspect, the present invention provides for ion-selective membrane devices comprising at least one membrane, optionally a hydrophobic membrane, at least one engineered ionophore of the present invention, and at least metal ion capable of binding with the at least one engineered ionophore. In one example embodiment, at any point in time, no metal ion is chelated with an engineered ionophore. In one example embodiment, at any point in time one or more or all metal ions is/are in the form of an ionophore metal ion chelate complex with an engineered ionophore.

In one example embodiment, the hydrophobic membrane of an ion-selective membrane device comprises a glass, a crystalline, a polymer membrane, or the like. In an embodiment, the ion-selective electrode membranes are ionophore-doped membranes. In an aspect, electivity of the ion-sensing membrane is conferred by the selection of the ionophore molecule.

In an embodiment, the ion-selective membrane comprising one or more ionophores according to the present description can be utilizes as an electrode. See, e.g., Gurzyninjski et al., Lead (II)-selective ionophores for ion-selective electrodes: A review Analytica Chimica Acta, Volume 791, 12 Aug. 2013, Pages 1-12; Buhlmann and Chen, Ion-Selective Electrodes with Ionophore-Doped Sensing Membranes, Supramolecule ChemistryL From Molecules to Nanomaterials (2012); Papp et al., Volume57, Issue17 Apr. 16, 2018, pages 4752-4755 (describing use of hydrophilic ionophores to construct ion-selective electrodes); Bondar, A. V., Keresten, V. M. & Mikhelson, K. N. Ionophore-Based Ion-Selective Electrodes in Non-Zero Current Modes: Mechanistic Studies and the Possibilities of the Analytical Application. J Anal Chem 77, 145-154 (2022); doi: 10.1134/S1061934822020046.

### METHODS OF USE

### Methods of Transporting Metal Ions Across Hydrophobic Membranes

In one aspect, the present invention provides for methods of increasing hydrophobic membrane transport of metal ions. In one example embodiment, a method comprises: forming a reaction mixture comprising: a metal ion; and an engineered ionophore of the present invention, wherein a chelate complex is formed; and contacting a reaction mixture with a hydrophobic membrane, wherein a chelate complex is transported across a hydrophobic membrane and a metal ion is released from a transported chelate complex, and wherein a chelate complex expresses increased hydrophobic membrane transport and/or metal ion release as compared to a counterpart chelate complex in the absence of a shielding group(s).

### Methods of Transporting Metal Ions Across Biological Membranes

In one example embodiment, a hydrophobic membrane is a biological membrane. In one example embodiment, a biological membrane is selected from cellular membranes, mitochondrial membranes, granule membranes (such as, for example insulin granules), other membrane which are inside cells, or cell wall (such as for example, in plants or bacteria).

In one example embodiment, a biological membrane is a cellular membrane, wherein: when a reaction mixture is formed and contacted with a cellular membrane outside of a cell, a vesicle, or a cellular organelle, a chelate complex is transported across a cellular membrane into a cell, a vesicle, or a cellular organelle; and when a reaction mixture is formed and contacted with a cellular membrane inside of a cell, a vesicle, or a cellular organelle, a chelate complex is transported across a cellular membrane and out of a cell, the vesicle, or a cellular organelle.

### Methods of Treating Disease

In one example embodiment, the contacting the reaction mixture with a cellular membrane occurs in an human or a-human animal, a plant, or a part thereof, and wherein the method targets, diagnoses, treats, prevents, or any combination thereof, a current or potential disease, disease state, condition, disorder, side effect, or the like, or any combination thereof, of the human or a non-human animal, a plant, or a part thereof.

In one example embodiment, a current or potential disease, disease state, condition, disorder, side effect, or the like, or any combination thereof, is selected from a metal ion deficiency (e.g., iron deficient anemia), a metal ion overload (e.g., an iron overload disease), a microbial (e.g., bacterial, viral, fungal, parasitic, or any combination thereof) infection or imbalance (e.g. dandruff), cancer, chronic kidney disease, Alzheimer's disease, and amyotrophic lateral sclerosis (ALS, also known as Lou Gehrig's disease).

In one example embodiment, a current or potential disease, disease state, condition, disorder, side effect, or the like, or any combination thereof, is a microbial infection, and wherein the method increases antimicrobial activity against the infection in the individual. In one example embodiment, the antimicrobial activity is selected from antibacterial activity, antiviral activity, antifungal activity, antiparasitic activity, or any combination thereof.

In one example embodiment, a method further comprises, prior to, concurrently with, or after forming the reaction mixture, administering the pharmaceutical composition of the present invention to a human or the animal, a plant, or a part thereof.

### Methods of Transporting Metal Ions Across Non-Biological Membranes

### Ion-Selective Membranes

In one example embodiment, a method occurs within the ion-selective membrane device of the invention.

Further embodiments are illustrated in the following Examples which are given for illustrative purposes only and are not intended to limit the scope of the invention.

### EXAMPLES

### Example 1—Rational Design and Validation of Potent Zinc Ionophores

Ionophores transport ions across biological membranes and have wide-ranging applications, but a platform for their rapid development does not exist. Applicants report a platform for developing ionophores from metal-ion chelators, which are readily available with wide-ranging affinities and specificities, and structural data that can aid rational design. Specifically, Applicants fine-tuned the binding affinity and lipophilicity of a Zn^{II}-chelating ligand by introducing silyl groups proximal to the Zn^{II}-binding pocket, which generated ionophores that performed better than most of the currently known Zn^{II} ionophores. Furthermore, these silicon-based ionophores were specific for Zn^{II} over other metals and exhibited better antibacterial activity and less toxicity to mammalian cells than several known Zn^{II} ionophores, including pyrithione. These studies establish rational design principles for the rapid development of potent and specific ionophores and a new class of antibacterial agents.

Herein, Applicants describe an approach to rationally design Zn^{II} ionophores from chelators. Applicants examined crystal structures of bispicolyl-based Zn^{II}-chelators to rationally introduce hydrophobic groups (e.g., silyl) proximal to the Zn^{II}-binding pocket to shield Zn^{II} and lower the binding affinity. The large size and hydrophobicity of silicon (vs. carbon) together with the readily available, wide assortment of silyl groups enabled rapid generation of ionophores selective for Zn^{II} over other metals [e.g., MgII, CaII, FeII, MnII]. Furthermore, these silicon-based ionophores were more potent than the known ionophores and exhibited potent antibacterial activity with lower mammalian cell toxicity. For example, while pyrithione was more toxic to mammalian cells over bacterial cells (0.1-fold difference), the silyl-based ionophores exhibited 7-fold selective toxicity to bacterial cells over mammalian cells. Overall, the platform for rapidly generating potent ionophores should have wide-ranging applications as both tools for studying cellular biology and as therapeutics against several key indications.

A general approach to rationally design Zn^{II} ionophores from Zn^{II} chelator by incorporation of silyl-based groups is reported. These ionophores were more potent than several reported Zn^{II} ionophores. Furthermore, these Zn^{II} ionophores demonstrated selective antibacterial activity and lower mammalian cell toxicity compared to the known ionophore, pyrithione.

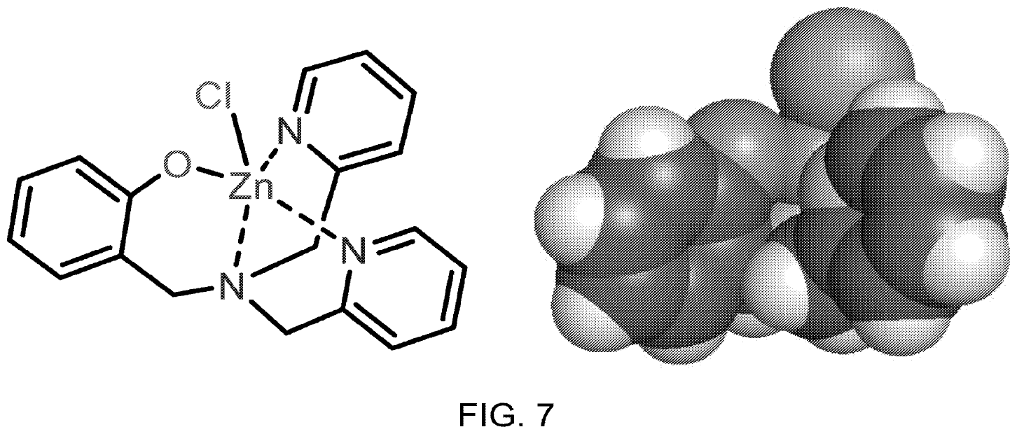

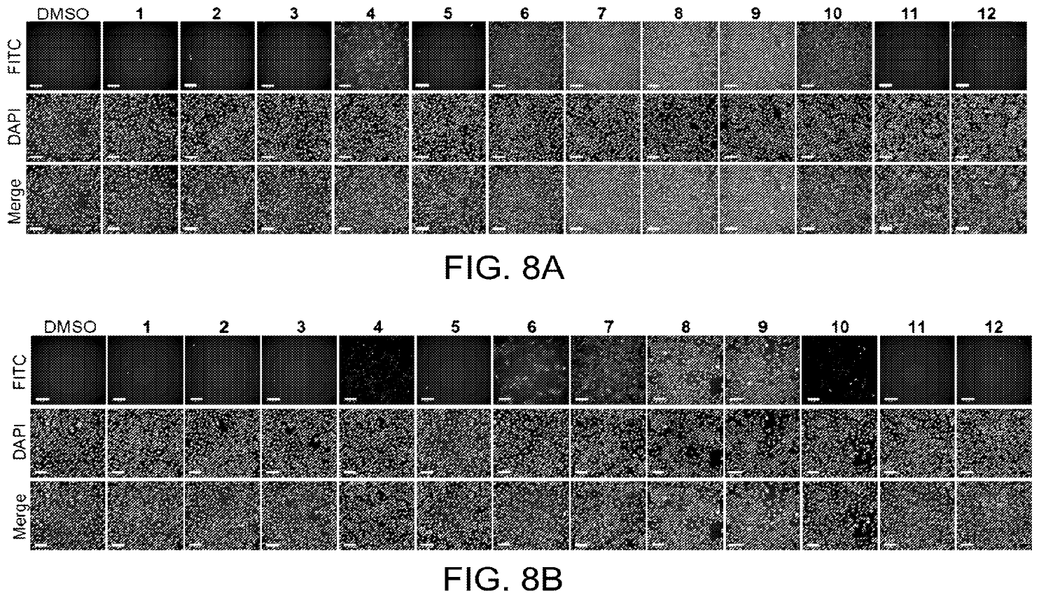

Crystal structure analysis of a bispicolyl-based Zn^{II} chelator (FIG. 7) [11] suggested that introducing a hydrophobic group proximal to the metal-ligand coordination pocket (e.g., ortho-position) would shield Zn^{II} and attenuate binding affinity.

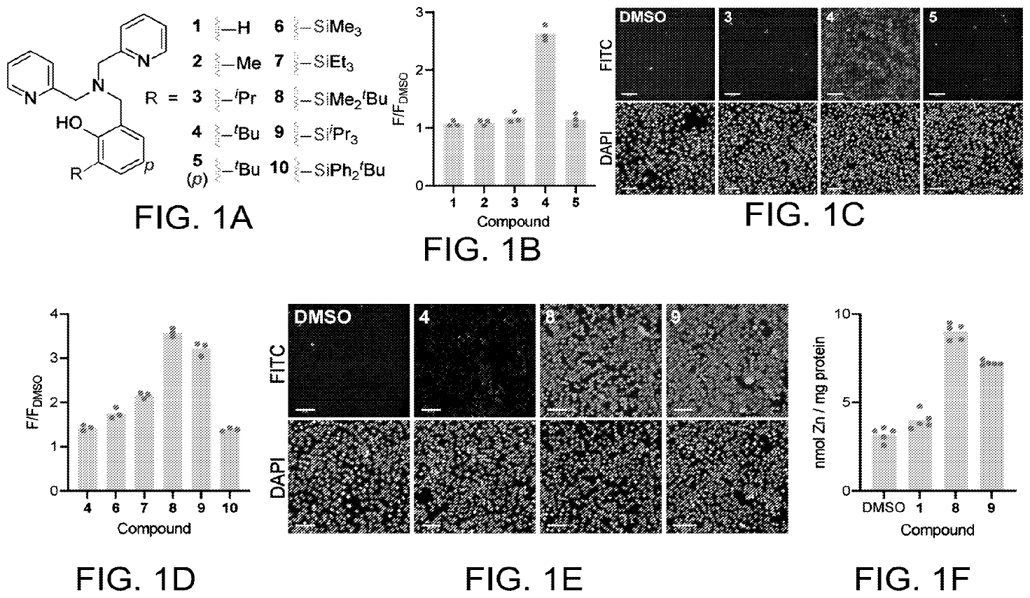

A number of bispicolyl-based Zn^{II}-chelators were synthesized with increasing size at the ortho-position of phenolic OH by a one-step reductive amination between bispicolyl-amine and commercially available 3-substituted salicylaldehydes. Applicants examined the ionophore activity of bispicolyl analogs with methyl (Me, 2), isopropyl (iPr, 3), and tert-butyl (tBu, 4) substitutions (FIG. 1A). Applicants tested the ionophore activity of these analogs using a Zn^{II}. selective sensor, Zinpyr-1 diacetate (DA-ZP1), [12] in U2OS cells whose cytosolic Zn^{II} concentration (˜400 pM) is below the detection limit of DA-ZP1. [13] The cells were first treated with ZnCl2 and ionophore candidates followed by DA-ZP1 treatment and fluorescence measurement. Applicants observed that only the tBu (4) analog efficiently transported Zn^{II} (FIG. 1B, C, and FIG. 8). Gratifyingly, the distal para-substituted tBu analog (5, FIG. 1A) that should not shield Zn^{II} or analogs with polar substitutions (e.g., OMe, Br) at the ortho position exhibited poor ionophore activity, as expected from the design principles (FIG. 1B, C, and FIG. 8).

Applicants hypothesized that Applicants could improve ionophore activity by introducing silyl groups at the ortho position. Due to the increased size, hydrophobicity, and lipophilicity of silicon relative to carbon, introducing a silyl group offers multiple advantages over its carbon isostere. Additionally, the widespread availability of different silyl-based protecting groups provides the option for fine-tuning the sizes and hydrophobicity of silyl-substitution. Applicants installed silyl groups at the ortho position via a two-step synthetic approach (Scheme S1 and S2). A number of 2-hydroxybenzaldehyde analogs containing 3-silyl groups were prepared by silylation of 2-bromophenol via retro-Brook rearrangement followed by Lewis-acid-mediated formylation using paraformaldehyde. [15]

Reductive amination of 3-silyl-substituted 2-hydroxy-benzaldehyde with bispicolylamine provided ionophores analogs with various silyl groups [TMS (6), TES (7), TBMS-(8), TIPS (9), TBDPS (10)].

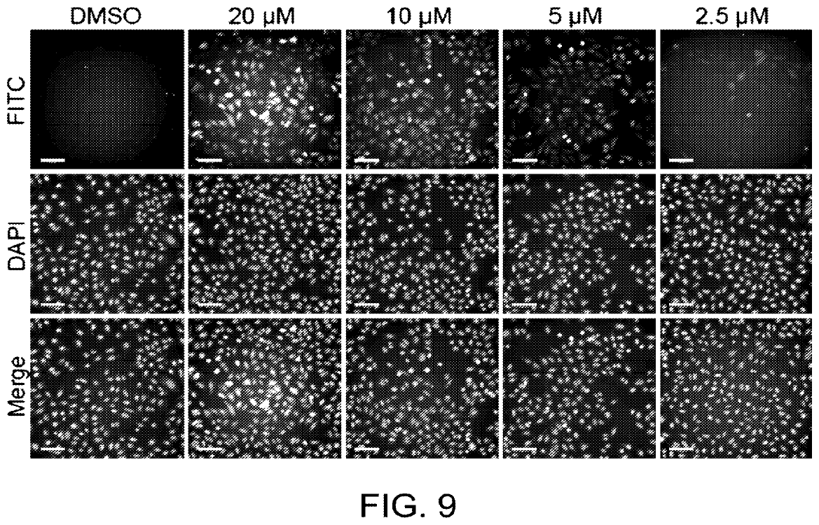

Applicants experimentally determined the ionophore activity of these silyl-based compounds using the aforementioned DAZ-P1 assay in U2OS cells. Gratifyingly, 6 containing a TMS group elicited detectable fluorescence at 5 μM, whereas its bioisosteric carbon-containing analog (tBu, 4) did not (FIG. 1D, E, and FIG. 8). Analogs with alkyl-containing silyl groups (e.g., TBMS in 8, TIPS in 9) improved ionophore activity, while phenyl-containing silyl groups (e.g., TDBPS in 10) decreased the activity. Applicants confirmed ionophore activity with another Zn^{II}-detecting dye, FluoZin-3, to ensure that the observed activity was not an artifact of the DA-ZP1-based assay (FIG. 9).

Next, Applicants used inductively coupled plasma mass spectrometry (ICP-MS) to validate the imaging assay and the ionophores activity (FIG. 1F). While the intracellular Zn^{II} levels where similar between DMSO and chelator 1, Zn^{II} concentrations inside cells were 3-fold higher when Applicants co-treated ionophores 8 and 9.

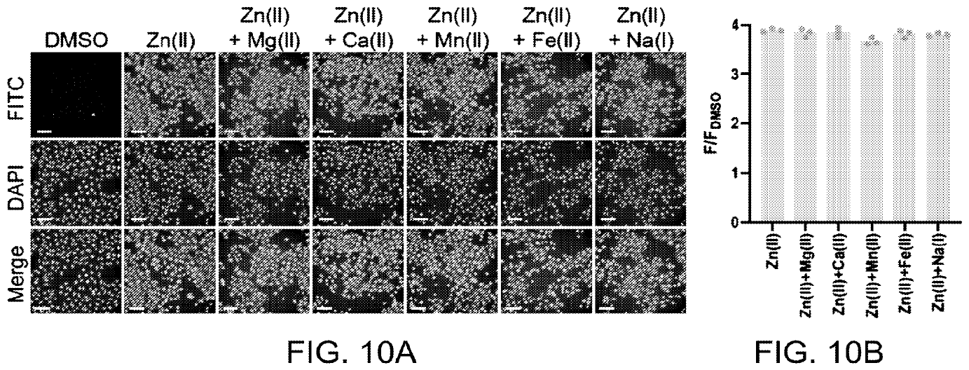

Applicants also determined metal-ion interference of the silyl-based ionophores with focused efforts on 8. Applicants designed a competition assay wherein Applicants pre-treated equimolar amounts of various metal ions, Mg^{II}, Ca^{II}, Mn^{II}, Fe^{II}, and Na^{I}, with 10 μM of Zn^{II} for 1 hour before treatment with DA-ZP1. Applicants did not observe significant drops in average fluorescence intensity for most metal ions in this competition assay, which indicates these metal ions did not interfere with ionophore activity (FIG. 10).

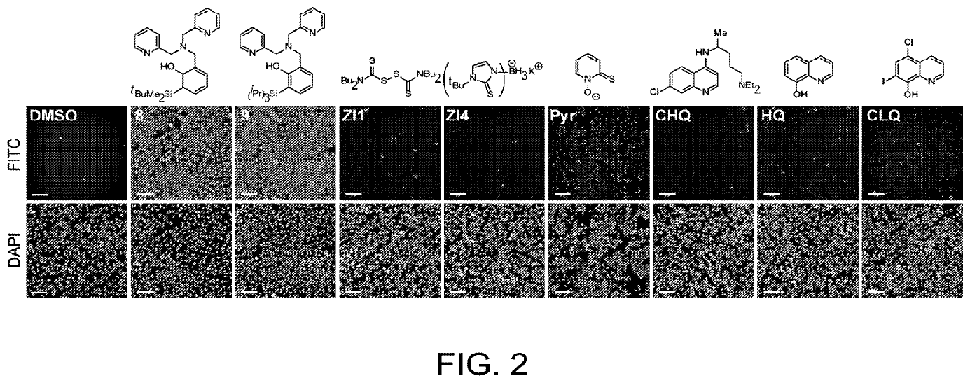

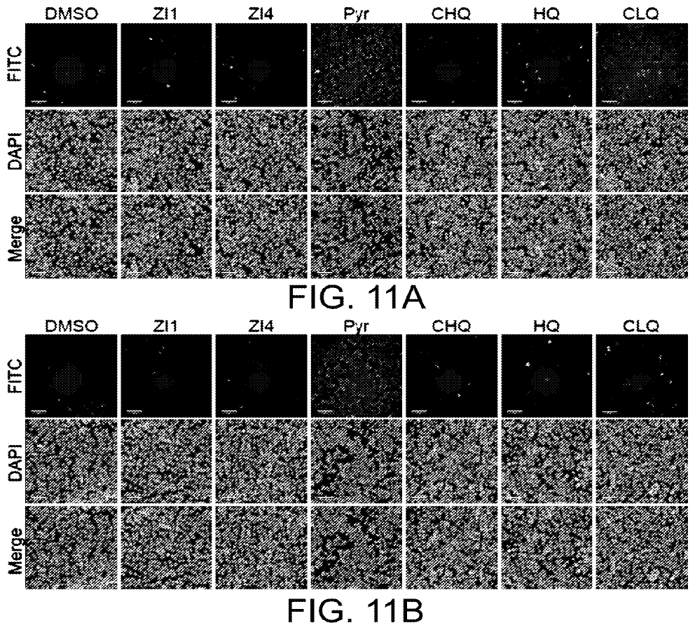

Finally, Applicants compared the activity of the designed compounds against known ionophores (FIGS. 2 and 11). [3,16] While the silyl-based ionophores showed activity in low micromolar concentrations (5 μM), only pyrithione and potassium hydrotris(N-tert-butyl-2-thioimidazolyl) borate also displayed activity at 5 μM, which was comparable to the two best ionophores, 8 and 9. Tetrabutylthiuram disulfide, clioquinol, and 8-hydroxyquinoline required >40 μM, and the activity of chloroquine was undetectable. [16]

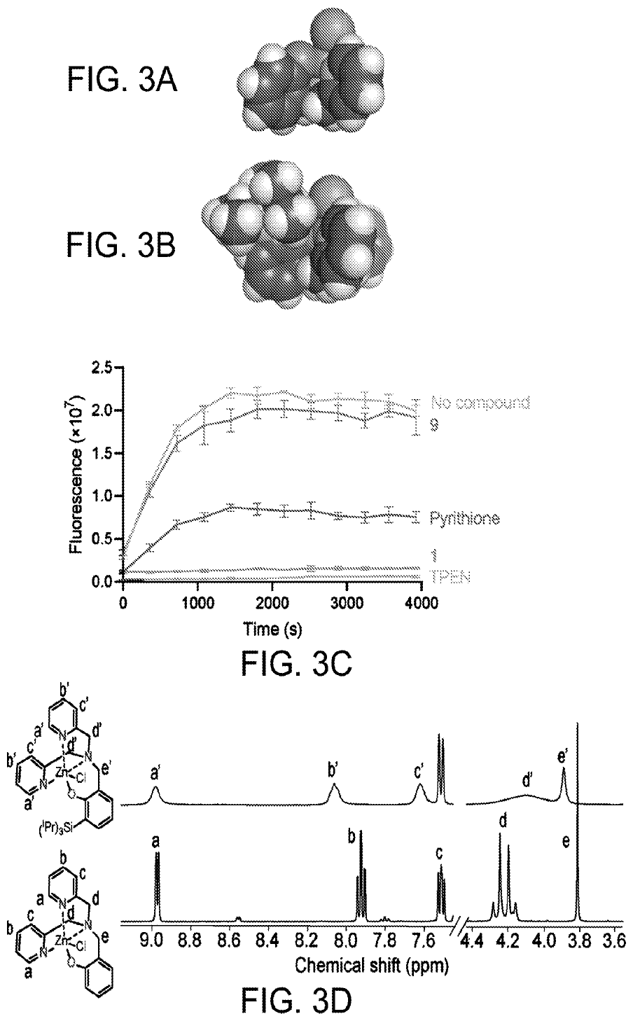

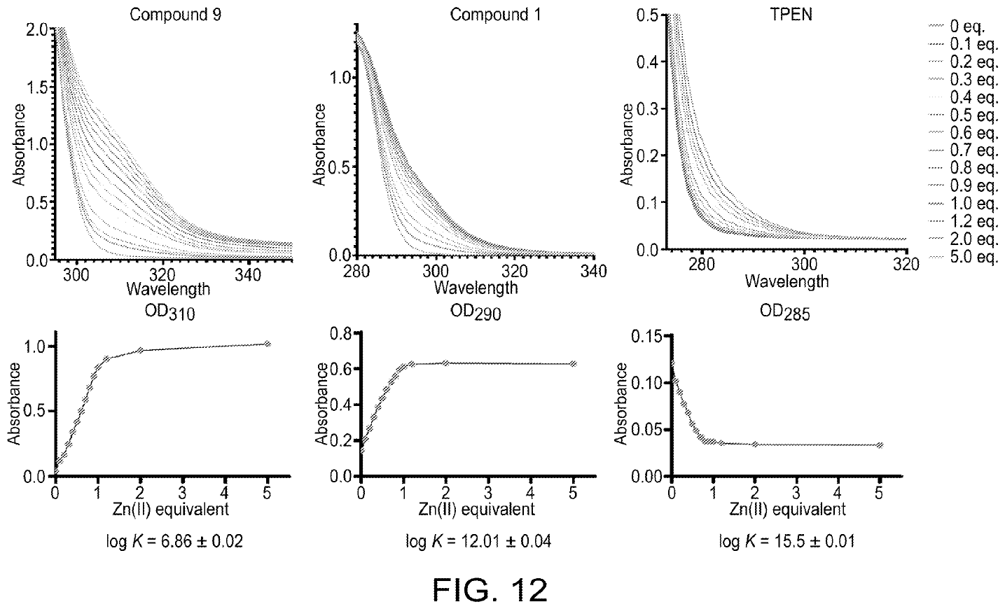

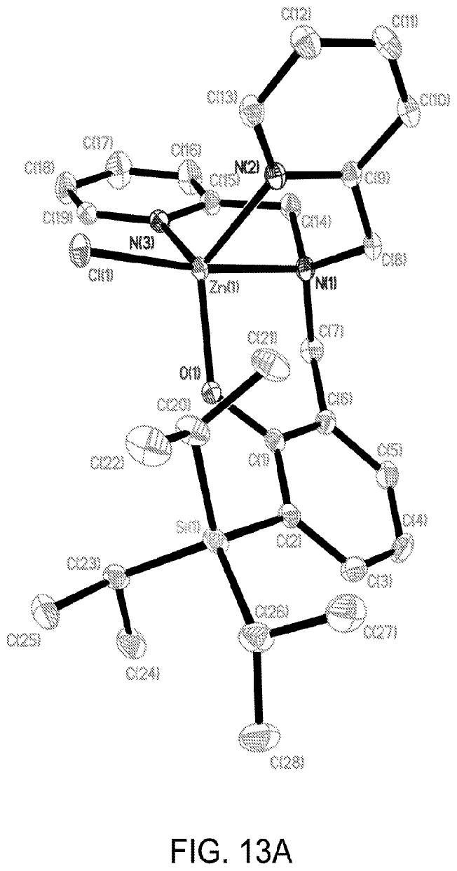

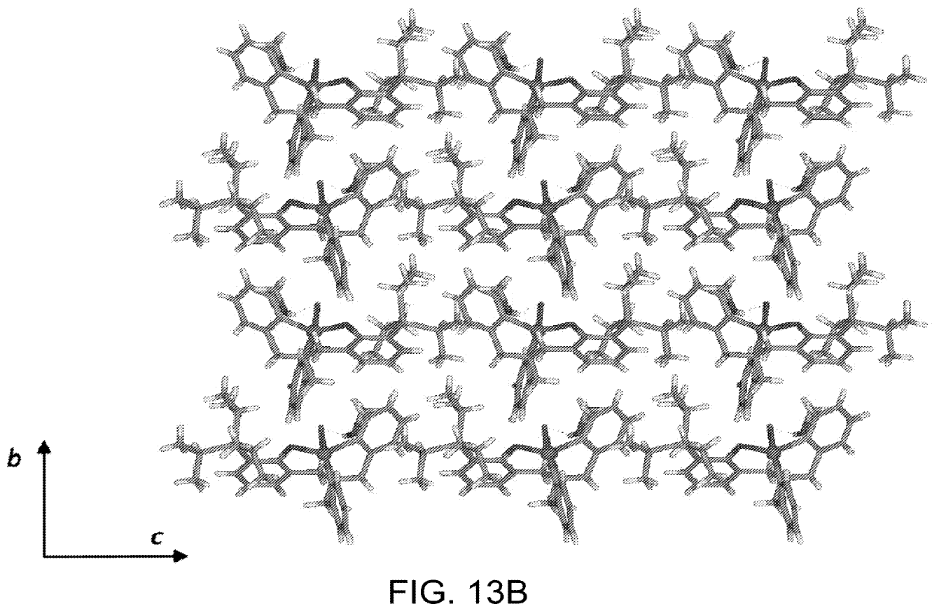

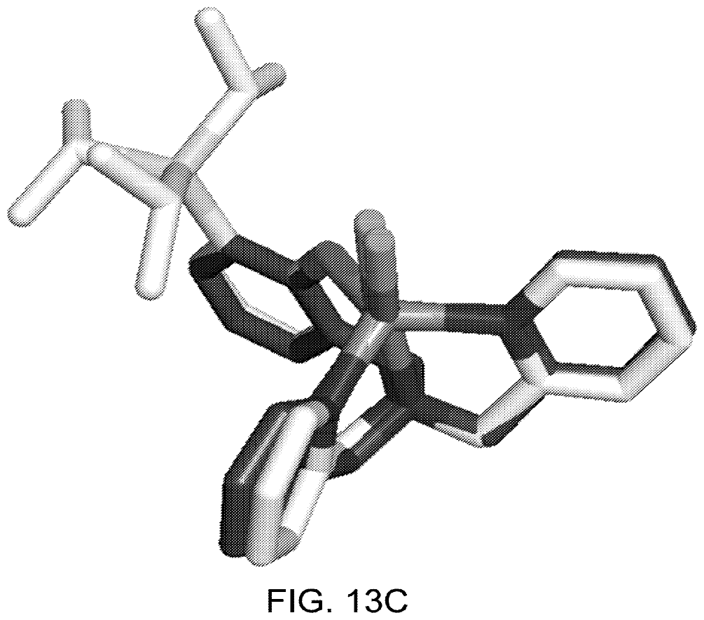

Applicants validated the design principles by confirming that the silyl group lowered the binding affinity and shielded the hydrophilic pocket. Using a UV/Visible spectroscopy-based titration assay, Applicants confirmed the silyl group lowered binding affinity: logK of 1 is 12.5, which is comparable to the known chelator TPEN (15.5) (FIG. 12). [17] The logK gradually decreased with the incorporation of a bulkier group, and logK values were calculated to be 7.5 and 6.9 in 8 and 9, respectively. Applicants confirmed that all compounds bound Zn^{II} with 1:1 stoichiometry (FIG. 12). An X-ray crystallography structure [18] of the Zn^{II}:9 showed greater shielding of the hydrophilic pocket that binds to Zn^{II} relative to the Zn^{II}:1 complex (FIG. 3A, B, and FIG. 13). Applicants superimposed the X-ray structure of the TIPS-containing Zn^{II}:9 complex with the literature precedented Zn^{II}:1 and observed significant structural distortion in the Zn^{II}:9 complex, which could explain the lower logK. [19]

Applicants also demonstrated that 9 released Zn^{II} more efficiently than 1, TPEN, or pyrithione. Briefly, Applicants co-incubated 10 μM of these compounds with 10 μM of Zn^{II} followed by the addition of 5 μM of DA-ZP1, which reacted rapidly with Zn^{II} (t_{1/2}=8.1 s) to generate fluorescence. Applicants then determined the relative rates of ligand exchange (FIG. 3C). Applicants observed a rapid increase in fluorescence for 9, which plateaued at the same level as that of 10 μM of Zn^{II}, but not for TPEN or 1, which suggested that 9 can more readily ligand exchange with DA-ZP1. Applicants also observed that pyrithione (log K=9.7) had a lower rate of ligand exchange. For more insight into the dynamics of Zn^{II}:1 or 9 complexation, Applicants performed an NMR experiment that showed a significant broadening of the 1H peak of Zn^{II}:9 as compared Zn^{II}:1 (FIG. 3D), suggesting higher fluxionality for the Zn^{II}:9 complex.

While these studies focused on the transport of extracellular zinc, Applicants hypothesized that these ionophores could also transport endogenous Zn^{II} between cellular compartments. Insulin granules in beta cells have an unusually high concentration of Zn^{II} (30 mM, 100 μM of which is “loosely bound”). [13,21] Applicants previously showed that DA-ZP1 selectively fluoresces inside beta cells relative to other cells in the islets of Langerhans and used this selective release of fluorescence to efficiently sort beta cells from human islets or other undesired cells that arise from directed differentiation of stem cells to beta cells. Applicants envisioned that the DA-ZP1 based fluorescence would increase if the ionophores could transport Zn^{II} from the insulin granule to the cytoplasm.

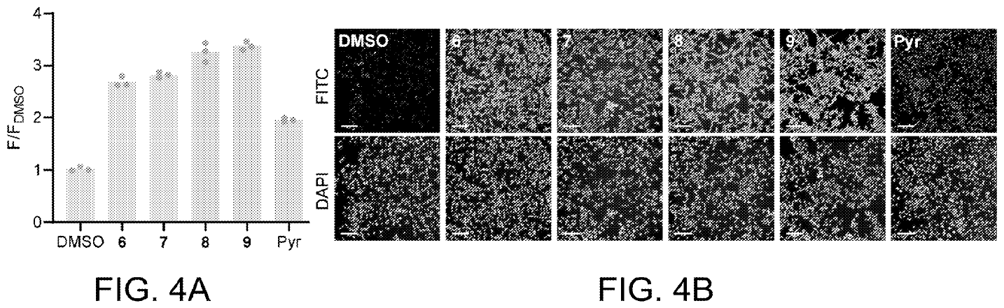

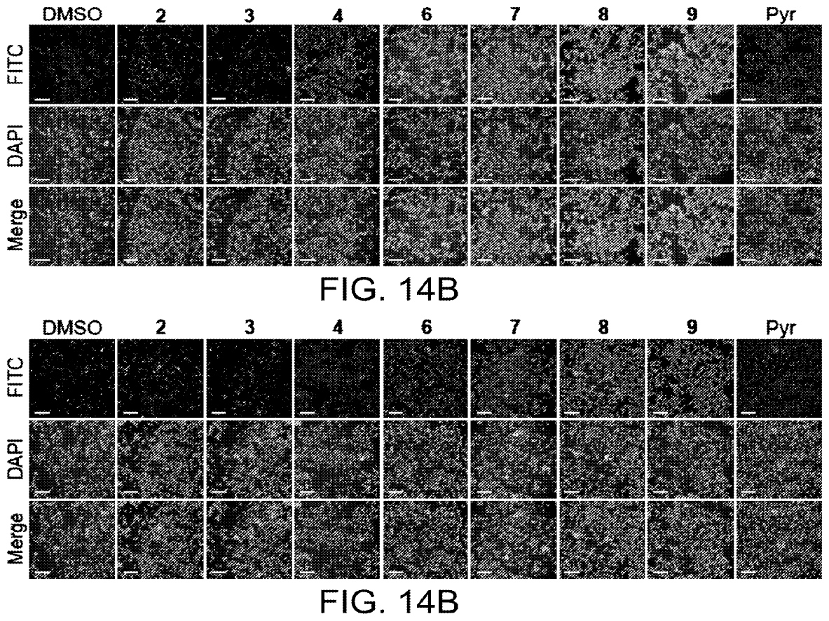

To confirm this, Applicants co-incubated the ionophores and DA-ZP1 for 1 hour before washing the beta cells and imaging. The ionophores 6 to 9 enhanced DA-ZP1 fluorescence intensity at a concentration of 10-20 μM (FIG. 4A, B, and FIG. 14), which is higher than that of pyrithione. Thus, these ionophores enhance the fluorescence of DA-ZP1 in beta cells and could further improve current beta-cell sorting protocols employed for both basic research and clinical purposes.

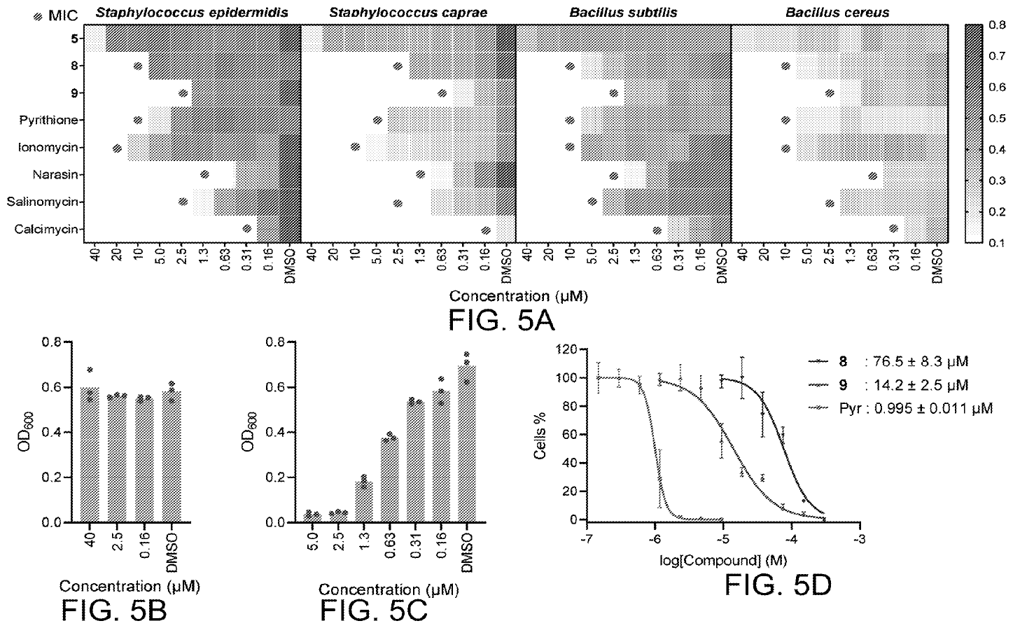

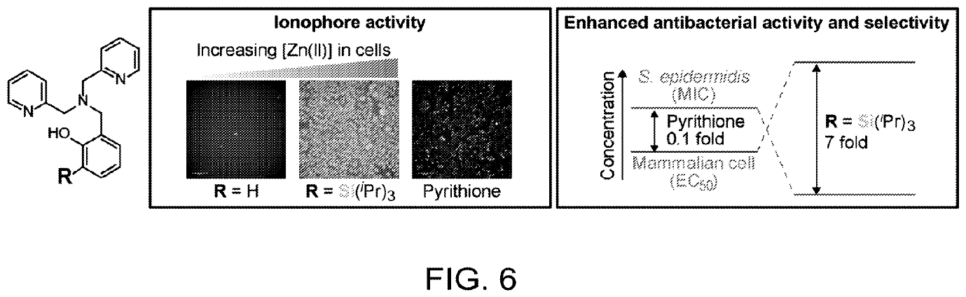

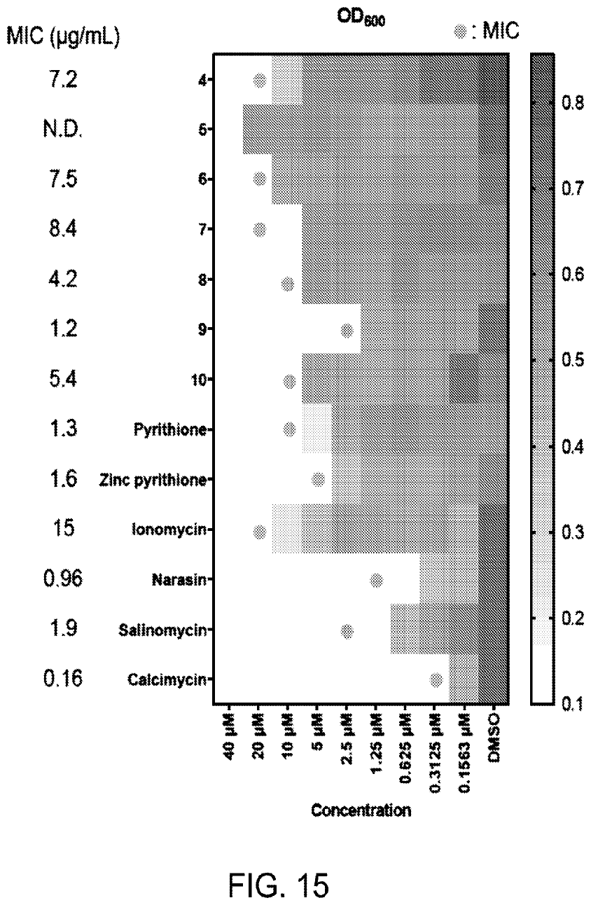

Next, Applicants evaluated the antibacterial activities of the ionophores. [7,8] First, Applicants determined their minimum inhibitory concentration (MIC) in the gram-positive bacterium that causes hospital-acquired infections, Staphylococcus epidermidis. [23] Compound 4 has potent antibacterial activity (MIC of 7.2 μgmL-1) (FIG. 5A and FIG. 15), which is better than the antibiotic ionomycin (MIC of 15 μg mL-1). Silyl-bearing analogs 6-10 exhibited higher antibiotic activity than 4 with estimated MICs of 7.5, 8.4, 4.2, 1.2, and 5.4 μg mL-1, respectively. The MIC of the best performing ionophore (9) was within the range of the known ionophores narasin (MIC 0.96 μgmL-1), salinomycin (MIC 1.88 μgmL-1), and pyrithione (MIC 1.3 μgmL-1). Overall, the known ionophore calcimycin had the highest antibacterial activity (MIC estimated at 0.16 μg mL-1). As a control, Applicants also evaluated 5, which showed no ionophore activity in U2OS cells or antibacterial activity. Applicants demonstrated antibacterial activity in multiple gram-positive strains (Staphylococcus caprae, Bacillus subtilis, and Bacillus cereus) with potencies comparable to those of known ionophores (FIG. 5A). The average MIC was 1.0 μg mL-1 and 1.1 μg mL-1 for 9 and pyrithione, respectively (Table 4).

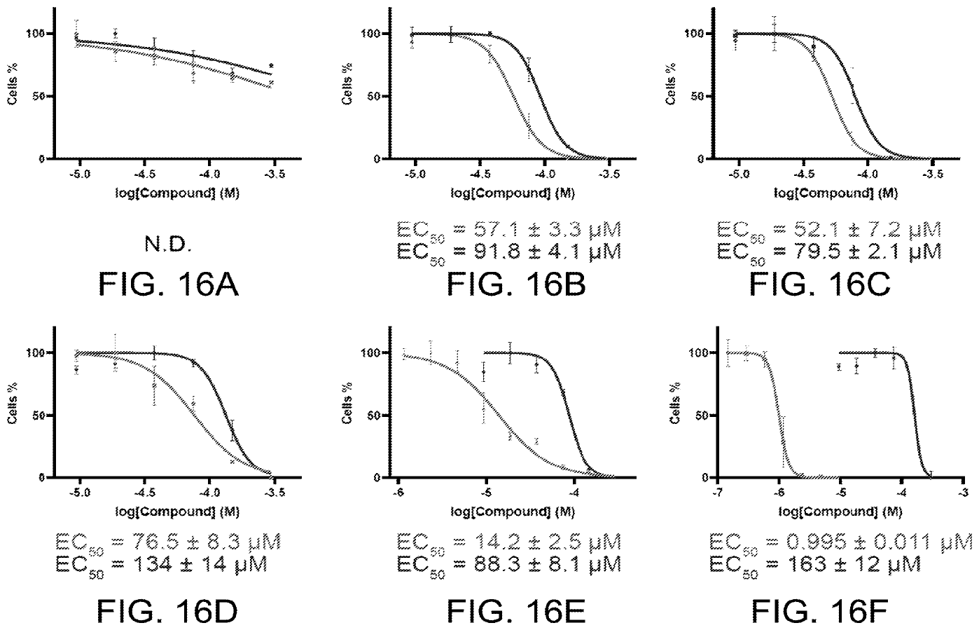

To confirm that the observed antibacterial activity was from Zn^{II} transport, Applicants performed experiments in the presence of the chelator EDTA. Applicants reasoned that limiting extracellular zinc with EDTA would abolish ionophore activity by depleting the available metal ions for membrane transport. Applicants co-incubated 50 μM of EDTA with various concentrations of 9 (FIG. 5B) and found that the MIC of 9 was dramatically increased with 50 μM of EDTA. Interestingly, the MIC was unaltered with additional Zn^{II} in the media, which suggested that the basal amount of Zn^{II} present was sufficient for antibacterial activity (FIG. 5C). [24] Applicants also observed this effect with pyrithione (FIG. 15). While the MIC values were comparable, the silyl-based ionophores exhibited significantly less toxicity to mammalian cells (Table 4, FIG. 5D and FIG. 16). The ionophores 8 and 9 had antibacterial selectivity of 9.4 and 6.8 times, respectively, compared to 0.11 for pyrithione.

TABLE 4
MICs of relevant ionophores against various bacteria and antibacterial selectivity.^{[a]}

| | | | | | | U2OS EC₅₀ + | |
|---|---|---|---|---|---|---|---|
| | Strain MIC [μg · mL⁻¹] | | | | Average MIC | 5 μM Zn^{II} | Antibacterial |
| Compound | S. epidermidis | S. caprae | B. subtilis | B. cereus | [μg · mL⁻¹] | [μM] | Selectivity |
| 8 | 4.2 | 1.1 | 4.2 | 4.2 | 3.4 (8.1 μM) | 76.5 | 9.4 |
| 9 | 1.2 | 0.3 | 1.2 | 1.2 | 0.98 (2.1 μM) | 14.2 | 6.8 |
| Pyrithione | 1.3 | 0.6 | 1.3 | 1.3 | 1.1 (8.7 μM) | 0.99 | 0.11 |

^{[a]}MIC data are the averages of two biological replicates, each performed in technical triplicates (distinct wells). The antibacterial selectivity is the ratio of the average molar MIC values in bacteria to the average EC₅₀ values in U2OS cells.

Overall, Applicants have generated a platform for the design of metal ionophores starting from metal chelators. Within this platform, Applicants used silicon to simultaneously fine-tune the binding affinity and lipophilicity of known metal chelators, a class of highly polar compounds, to endow them with cell permeability and metal-releasing capabilities (i.e., ionophore activity). The designed Zn^{II} ionophores exhibited potent antibacterial activity and selectivity over mammalian cells, comparable to known antibiotics and ionophores, despite their structural simplicity. Applicants envision the implementation of these design principles to other metal ions for which chelators with wide-ranging affinities and specificities are readily available. For example, several chelators of iron are available and iron ionophores can aid or establish homeostasis in disease conditions. [1a] On the whole, these studies expand the chemical space and concomitant biological applicability of ionophores.

### REFERENCES

[1] a) A. Steinbrueck, A. C. Sedgwick, J. T. Brewster II, K. C. Yan, Y. Shang, D. M. Knoll, G. I. Vargas-Zuniga, X. P. He, H. Tian, J. L. Sessler, Chem. Soc. Rev. 2020, 49, 3726-3747; b) H. Liu, S. Lin, K. M. Jacobsen, T. B. Poulsen, Angew. Chem. Int. Ed. 2019, 58, 13630-13642; Angew. Chem. 2019, 131, 13764-13777; c) V. Oliveri, Coord. Chem. Rev. 2020, 422, 213474-213498; d) M. E. Helsel, K. J. Franz, Dalton Trans. 2015, 44, 8760-8770; e) D. A. Kevin Ii, D. A. Meujo, M. T. Hamann, Exp. Opin. Drug Discovery 2009, 4, 109-146; f) A. Huczyński, Bioorg. Med. Chem. Lett. 2012, 22, 7002-7010; g) A. Roy, P. Talukdar, ChemBioChem 2021, 22, 2925-2940; h) M. S. An-toszczak, A. Huczyński, Euro. J. Med. Chem. 2019, 166, 32-47.
[2] a) S. Lin, H. Liu, E. B. Svenningsen, M. Wollesen, K. M. Jacobsen, F. D. Andersen, J. Moyano-Villameriel, C. N. Peder-sen, P. Norby, T. Torring, T. B. Poulsen, Nat. Chem. 2021, 13, 47-55; b) T. A. Su, D. S. Shihadih, W. Cao, T. C. Detomasi, M. C. Heffern, S. Jia, A. Stahl, C. J. Chang, J. Am. Chem. Soc. 2018, 140, 13764-13774; c) S. Tardito, I. Bassanetti, C. Bignardi, L. Elviri, M. Tegoni, C. Mucchino, O. Bussolati, R. Franchi-Gazzola, L. Marchio, J. Am. Chem. Soc. 2011, 133, 6235-6242; d) S. Tardito, A. Barilli, I. Bassanetti, M. Tegoni, O. Bussolati, R. Franchi-Gazzola, C. Mucchino, L. Marchio, J. Med. Chem. 2012, 55, 10448-10459; e) R. M. Vaden, K. P. Guillen, J. M. Salvant, C. B. Santiago, J. B. Gibbons, S. S. Pathi, S. Arunachalam, M. S. Sigman, R. E. Looper, B. E. Welm, ACS Chem. Biol. 2019, 14, 106-117; f) D. Magda, P. Lecane, Z. Wang, W. Hu, P. Thiemann, X. Ma, P. K. Dranchak, X. Wang, V. Lynch, W. Wei, V. Csokai, J. G. Hacia, J. L. Sessler, Cancer Res. 2008, 68, 5318-5325.
[3] P.-J. Sun, Q. Fernando, H. Freiser, Anal. Chem. 1964, 36, 2485-2488.
[4] P. Chandrangsu, C. Rensing, J. D. Helmann, Nat. Rev. Micro-biol. 2017, 15, 338-350.
[5] S. H. Choi, K. L. Lee, J. H. Shin, Y. B. Cho, S. S. Cha, J. H. Roe, Nat. Commun. 2017, 8, 15812.
[6] C. Andreini, L. Banci, I. Bertini, A. Rosato, J. Proteome Res. 2006, 5, 196-201.
[7] a) N. Harbison-Price, S. A. Ferguson, A. Heikal, G. Taiaroa, K. Hards, Y. Nakatani, D. Rennison, M. A. Brimble, I. M. El-Deeb, L. Bohlmann, C. A. McDevitt, M. von Itzstein, M. J. Walker, G. M. Cook, mSphere 2020, 5, e00157-20; b) L. Bohlmann, D. M. P. De Oliveira, I. M. El-Deeb, E. B. Brazel, N. Harbison-Price, C. Y. Ong, T. Rivera-Hernandez, S. A. Ferguson, A. J. Cork, M. D. Phan, A. T. Soderholm, M. R. Davies, G. R. Nimmo, G. Dougan, M. A. Schembri, G. M. Cook, A. G. McEwan, M. von Itzstein, C. A. McDevitt, M. J. Walker, mBio 2018, 9, e02391-18.
[8] D. M. P. De Oliveira, L. Bohlmann, T. Conroy, F. E. Jen, A. Everest-Dass, K. A. Hansford, R. Bolisetti, I. M. El-Deeb, B. M. Forde, M. D. Phan, J. A. Lacey, A. Tan, T. Rivera-Hernandez, S. Brouwer, N. Keller, T. J. Kidd, A. J. Cork, M. J. Bauer, G. M. Cook, M. R. Davies, S. A. Beatson, D. L. Paterson, A. G. McEwan, J. Li, M. A. Schembri, M. A. T. Blaskovich, M. P. Jennings, C. A. McDevitt, M. von Itzstein, M. J. Walker, Sci. Transl. Med. 2020, 12, eabb3791.
[9] P. N. Basa, C. A. Barr, K. M. Oakley, X. Liang, S. C. Burdette, J. Am. Chem. Soc. 2019, 141, 12100-12108.
[10] C. R. Goldsmith, S. J. Lippard, Inorg. Chem. 2006, 45, 555-561.
[11] H. Adams, N. A. Bailey, D. E. Fenton, Q.-Y. He, J. Chem. Soc. Dalton Trans. 1996, 2857-2865.
[12] W. Chyan, D. Y. Zhang, S. J. Lippard, R. J. Radford, Proc. Natl. Acad. Sci. USA 2014, 111, 143-148.
[13] M. C. Carpenter, M. N. Lo, A. E. Palmer, Arch. Biochem. Biophys. 2016, 611, 20-29.
[14] a) A. K. Franz, S. O. Wilson, J. Med. Chem. 2013, 56, 388-405; b) S. Fujii, MedChemComm 2016, 7, 1082-1092; c) R. Ramesh, D. S. Reddy, J. Med. Chem. 2018, 61, 3779-3798.
[15] a) T. Yoshimura, K. Tomohara, T. Kawabata, J. Am. Chem. Soc. 2013, 135, 7102-7105; b) T. N. T. Ikawa, T. Nosaki, A. Takagi, S. I. Akai, Org. Lett. 2011, 13, 1730-1733.
[16] J. Xue, A. Moyer, B. Peng, J. Wu, B. N. Hannafon, W. Q. Ding, PLOS One 2014, 9, e109180.
[17] M. L. Zastrow, R. J. Radford, W. Chyan, C. T. Anderson, D. Y. Zhang, A. Loas, T. Tzounopoulos, S. J. Lippard, ACS Sens. 2016, 1, 32-39.
[18] Deposition Number 2112403 contains the supplementary crys-tallographic data for this paper. These data are provided free of charge by the joint Cambridge Crystallographic Data Centre and Fachinformationszentrum Karlsruhe Access Structures service.
[19] a) F. Basolo, R. K. Murmann, J. Am. Chem. Soc. 1952, 74, 5243-5246; b) A. Miličević, N. Raos, J. Phys. Chem. A 2008, 112, 7745-7749.
[20] M. Park, Y. J. Cho, Y. W. Lee, W. H. Jung, Sci. Rep. 2018, 8, 12086.
[21] Y. V. Li, Endocrine 2014, 45, 178-189.
[22] a) M. Lee, B. Maji, D. Manna, S. Kahraman, R. M. Elgamal, J. Small, P. Kokkonda, A. Vetere, J. M. Goldberg, S. J. Lippard, R. N. Kulkarni, B. K. Wagner, A. Choudhary, J. Am. Chem. Soc. 2020, 142, 6477-6482; b) K. Yang, M. Lee, P. A. Jones, S. S. Liu, A. Zhou, J. Xu, V. Sreekanth, J. L. Y. Wu, L. Vo, E. A. Lee, R. Pop, Y. Lee, B. K. Wagner, D. A. Melton, A. Choudhary, J. F. Karp, Sci. Adv. 2020, 6, eabc3207; c) S. Kahraman, D. Manna, E. Dirice, B. Maji, J. Small, B. K. Wagner, A. Choudhary, R. N. Kulkarni, Life Sci. Alliance 2021, 4, e202000840.
[23] M. Otto, Nat. Rev. Microbiol. 2009, 7, 555-567.
[24] A. Bilinskaya, D. J. Buckheit, M. Gnoinski, T. E. Asempa, D. P. Nicolau, J. Clin. Microbiol. 2020, 58, e02019-20.
[25] a) R. D. Hancock, Chem. Soc. Rev. 2013, 42, 1500-1524; b) E. L. Que, D. W. Domaille, C. J. Chang, Chem. Rev. 2008, 108, 1517-1549; c) K. L. Haas, K. J. Franz, Chem. Rev. 2009, 109, 4921-4960.

### Materials and General Procedures

### Materials

All reagents were purchased and used as received from commercial sources without further purification. Reactions were performed in round-bottom flasks stirred with Teflon®-coated magnetic stir bars. Moisture and air-sensitive reactions were performed under a dry nitrogen/argon atmosphere. Moisture and air sensitive liquids or solutions were transferred via nitrogen-flushed syringes. As necessary, organic solvents were degassed by bubbling in nitrogen or argon. The reaction progress was monitored by thin-layer chromatography (TLC) and ultra-performance liquid chromatography mass spectrometry (UPLC-MS). Flash column chromatography was performed using silica gel (60 Å mesh, 20-40 μm) on a Teledyne IscoCombiFlash Rf system. Analytical TLC was performed using Merck Silica gel 60 F254 pre-coated plates (0.25 mm); illumination at 254 nm allowed the visualization of UV-active material, and a phosphomolybdic acid (PMA) stain was used to visualize UV-inactive material. UPLC-MS was performed on a Waters ACQUITY UPLC I-Class PLUS System with an ACQUITY SQ Detector 2. Nuclear magnetic resonance (NMR) spectra were recorded on a Bruker 400 Spectrometer (¹H NMR, 400 MHz; ¹³C, 101 MHz) at the Broad Institute of MIT and Harvard. ¹H and ¹³C chemical shifts are indicated in parts per million (ppm) relative to SiMe4 (δ=0.00 ppm) and internally referenced to residual solvent signals. NMR solvents were purchased from Cambridge Isotope Laboratories, Inc., and NMR data were obtained in CDCl3 or DMSO. Data for ¹H NMR are reported as follows: chemical shift value in ppm, multiplicity (s=singlet, d=doublet, t=triplet, dd=doublet of doublets, and m=multiplet), integration value, and coupling constant value in Hz. Tandem liquid chromatography-mass spectrometry (LCMS) was performed on a Waters 2795 separations module with a 3100-mass detector. High-resolution mass spectra were recorded on a JEOL AccuTOF LC-Plus 46 DART system at the department of chemistry instrumentation facility at the Massachusetts Institute of Technology and a Thermo Q Exactive Plus mass spectrometer system equipped with an HESI-II electrospray ionization source at Harvard Center for Mass Spectrometry at the Harvard FAS Division of Science Core Facility. Tetrabutylthiuram disulfide (zinc ionophore 1, catalog no. 96491), potassium hydrotris(N-tert-butyl-2-thioimidazolyl) borate (zinc ionophore 4, catalog no. 74196), chloroquine phosphate (catalog no. 1118000), ionomycin (catalog no. 19657), narasin (catalog no. N1271), salinomycin (catalog no. S4526), and calimycin (catalog no. C7522) were purchased from Sigma Aldrich. Clioquinol (catalog no. OR-0033) was purchased from Combi-Blocks, and 8-hydroxyquinoline (catalog no. 093034) was purchased from Oakwood Products.

### General Procedure A. UV/Vis Titration Experiment

A solution of ligand was prepared in acetonitrile. The starting concentration was chosen to be around 0.10 mM, based on the absorption maximum of the compound. This solution (2.5 mL) was pipetted into a quartz cuvette with a 1-cm optical path length. The titration was performed by the stepwise addition of a 10-mM ZnC12 solution in acetonitrile. The absorption spectra were measured over the range of 250-800 nm. The calculation of the stability constants was accomplished using TitrationFit.1

### General Procedure B. Co-Incubation Kinetic Studies on DA-ZP1 in the Presence of Compound and Zn(II)

The compound in phosphate-buffered saline (PBS) was added to a 96-well microplate (250 μL, 10 μM per well, Nunc 96-well plates) and incubated with 10 μM of ZnCl₂. After 15 min, 10 μM of DA-ZP1 was added. Reaction kinetics were monitored by measuring the fluorescence of the activated fluorescein dye (λₑₓ=490 nm, λₑₘ=522 nm) over 2 h.

### General Procedure C. Cell Culture

U20S and HEK293T cells were cultured in a humidified 5% CO2 atmosphere at 37° C. U2OS cells (ATCC) were cultured in Dulbecco's Modified Eagle's Medium (DMEM, Life Technologies) supplemented with 10% fetal bovine serum (FBS; Life Technologies), 1X penicillin/streptomycin (Life Technologies), and 1X sodium pyruvate (Life Technologies). INS-1E cells were cultured in RPMI 1640 medium (Life Technologies) supplemented with 10% FBS, 1X penicillin/streptomycin, 1X sodium pyruvate (Life Technologies), and 3.5 μL/L β-mercaptoethanol (Sigma). Cells were continuously maintained at <90% confluency. All cell lines were sourced commercially.

### General Procedure D. Zinc Imaging Assay in U2OS Cells

In a 96-well plate, 20,000 U2OS cells were plated per well and cultured for 24 h in the incubator set to 37° C. with 5% CO₂. Compounds were added at the indicated concentration along with 50 M of zinc chloride in the cell culture medium, and together they were kept for 1 h in the incubator. The medium containing the compound was removed, and the cells were washed with 3×100 μL of fresh medium, and finally another 100 μL of 2 μM of DA-ZP1 was added to the corresponding cell culture medium containing HCS NuclearMask Blue Stain. The cells were again kept for 1 h in the incubator. The medium containing compound was removed, and the cells were washed with 1×100 μL of PBS and 2×100 μL of fresh medium, and finally, another 100 μL of fresh, dye-free medium was added. The cells were immediately imaged using a high-content fluorescence microscope (Operetta Phenix) in both the DAPI and FITC channels at 20× magnification. Images were analyzed using the Harmony software, and data were plotted using GraphPad Prism 9.

### General Procedure E. ICP-MS Analysis

U2OS cells (˜200,000) were plated in a 6-well dish. After a day, the medium was aspirated, and Zn(II) (10 μM) and compounds (5 μM) containing media (2 mL) were added to each appropriate well. Cells were harvested by first washing each well with PBS containing EDTA (100 μM, 2×2.0 mL) and subsequently adding Milli-Q water (18.2 M (2 cm, 1 mL) to each well and incubating for 2-4 min. A portion of the resulting cell lysate (900 μL) was collected and transferred to an Eppendorf Safe-Lock tube, snap-frozen in N₂(l), and then thawed at room temperature (RT) for a total of three freeze-thaw cycles. The insoluble lysate was pelleted (10 min at 10,000 g), and the soluble protein concentration of each sample was determined using the Pierce 660 nm Protein Assay in a 96-well plate (200 μL of reagent, 14 μL of sample, bovine serum albumin standard curve). Samples were then frozen and lyophilized overnight, and the resulting white, flaky solid was taken up in concentrated nitric acid (200 μL, ultrapure for trace metal analysis, BDH Aristar) and heated at 70° C. for 1 h. A portion of each sample (142 μL) was then diluted with water (2 mL) and a terbium internal standard (Agilent 5190-8590; stock at 10,000 parts per billion [ppb] in 10% nitric acid diluted to 100 ppb in Milli-Q water for addition to samples, 2 ppb final concentration), after which the metal content of the samples was assessed by ICP-MS. To calculate the molarities and the corresponding molar amounts of metals in samples, a density of 1.00 g/mL was assumed for all solutions. Therefore, 1 ppb was taken to be equal to 1 μg/L, and the molarity of a particular solution was considered equal to the measured concentration of a particular ion in ppb, divided by the molecular weight of the ion, multiplied by 10-6.

### General Procedure F. Zinc Imaging Assay in INS-1E Cells

In a 96-well plate, 30,000 INS-1E cells were plated per well and cultured for 24 h at 37° C. with 5% CO₂. Compounds were added at the indicated concentrations along with 2 μM of DA-ZP1 in the corresponding cell culture medium containing HCS NuclearMask Blue Stain, and together they were kept for 1 h in the incubator. The medium containing the compound was removed, and the cells were washed with 1×100 μL of PBS and 2×100 μL of fresh medium, and finally, another 100 μL of fresh, dye-free medium was added. The cells were immediately imaged using a high-content fluorescence microscope (Operetta Phenix) in both the DAPI and FITC channels at 20× magnification. Images were analyzed using the Harmony software and data were plotted using GraphPad Prism 9.

### General Procedure G. Cell Viability Assay

U2OS cells were seeded in black 384-well plates at a density of 3,000 cells per well in 25 μL of medium per well. After seeding, the cells were left to adhere overnight. The compounds to be tested were added to the designated culture plates in 25 μL of medium in triplicate with a normalized DMSO concentration (0.05%) in all treatments, and the plates were placed at 37° C. in a humidified atmosphere containing 5% CO₂. After 48 h, 40 μl of CellTiter-Glo (Promega) was added to each well and incubated for 30 min, after which the plate luminescence was analyzed in a PerkinElmer EnVision Manager 1.13 (PerkinElmer). The average growth of treated cells was calculated by correcting the values for background luminescence and was subsequently normalized to the average of the two lowest treatments of the compound in question. Data were plotted and fitted to a dose-response curve using GraphPad Prism 9.

### General Procedure H. Bacterial Strains, Media, and Growth Conditions

Staphylococcus epidermidis ATCC® 12228™, Staphylococcus caprae ATCC® 51549™, Bacillus subtilis ATCC® 6051™, and Bacillus cereus ATCC® 14579™ were grown in cation-adjusted Mueller-Hinton broth (CA-MHB) (catalog no. 90922, Sigma) as per the Clinical and Laboratory Standards Institute guidelines.2 Bacteria were routinely grown at 37° C. under aerobic conditions.

### General Procedure I: Evaluation of Antibacterial Spectra of Compounds

The MIC assay was performed according to the Clinical and Laboratory Standards Institute protocols.³ The antibacterial activities of the compounds were measured using the broth microdilution method. Serial dilutions of compounds were prepared in cation-adjusted Müller Hinton Broth. Each dilution (100 μL) was dispensed to each well of a round-bottom plate to obtain final concentrations ranging from 0.07815-40 μM. Bacteria were grown at 37° C. overnight on cation-adjusted Müller Hinton Broth. The bacterial solution was prepared in 0.9% saline and adjusted to obtain an OD₆₀₀=0.1 using a SpectraMax M5 (Molecular Devices). The resultant suspension was diluted 1:20 with cation-adjusted Müller Hinton Broth. The resulting mixture (10 μL) was added to each well of the plates (approximately 5×10⁴ CFU/well), mixed, and incubated at 37° C. for 24 h. The MIC value was determined as the minimum concentration that inhibited the growth of bacteria.

### General Procedure J: 2,2′-Bipicolyl-Based Ligand Synthesis

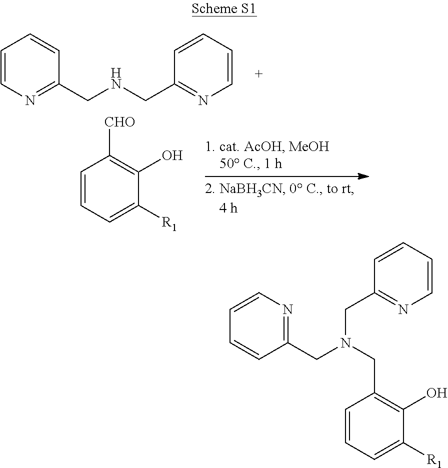

An oven-dried round-bottom with a stir bar was charged with 3-substituted salicylaldehyde derivatives (1 mmol) and di-picolylamine (100 mg, 1 mmol) in 20 mL of methanol followed by 100 μL of glacial acetic acid. The reaction mixture was heated at 50° C. for 1 h, and cooled down to 0° C. using an ice bath. NaBH₃CN (186 mg, 3 mmol, 3 equivalent) was carefully delivered to the reaction mixture, which was further stirred for 4 hours at room temperature. The reaction was quenched with water (20 mL), and after removing the volatiles, the residue was further dissolved in CH₂Cl₂ (3×50 mL) and water (50 mL). The recovered organic layer was dried over anhydrous Na₂SO₄, filtered, and concentrated. Silica gel column purification was performed with CH₂Cl₂:CH₃OH=19:1 to 9:1 (v/v) to afford the ligands 1-10.

2-((bis(pyridin-2-ylmethyl)amino)methyl)phenol (1) was prepared by following the general procedure J with 2-hydroxybenzaldehyde (122 mg, 1 mmol). The final product was isolated by flash column chromatography (DCM:MeOH 95:5, R_{f}=0.5) in 51% (156 mg) yield.₄

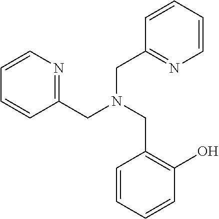 [SMILES: Oc1ccccc1CN(Cc1ccccn1)Cc1ccccn1]

2-((bis(pyridin-2-ylmethyl)amino)methyl)-6-methylphenol (2) was prepared following the general procedure J with 2-hydroxy-3-methylbenzaldehyde (136 mg, 1 mmol). The final product was isolated by flash column chromatography (DCM:MeOH 95:5, R_{f}=0.5) in 58% (185 mg) yield. ¹H NMR (400 MHZ, MeOH-d₄) δ 8.54-8.41 (m, 2H), 7.68 (td, J=7.7, 1.8 Hz, 2H), 7.37 (d, J=7.8 Hz, 2H), 7.21 (dd, J=7.5, 5.1 Hz, 2H), 6.97 (d, J=7.4 Hz, 1H), 6.88 (dd, J=7.5, 1.6 Hz, 1H), 6.63 (t, J=7.5 Hz, 1H), 3.78 (s, 4H), 3.70 (s, 2H), 2.19 (s, 3H). ¹³C NMR (101 MHZ, MeOH-d₄) δ 159.29, 156.36, 149.63, 138.48, 131.28, 128.80, 125.97, 124.76, 123.76, 123.25, 119.83, 60.02, 57.95, 16.19. HRMS m/z calcd for [M-H]⁻ 318.1612, obsd 318.1611.

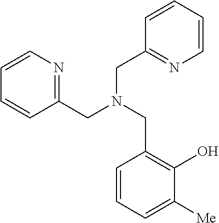 [SMILES: Cc1cccc(CN(Cc2ccccn2)Cc2ccccn2)c1O]

2-((bis(pyridin-2-ylmethyl)amino)methyl)-6-isopropylphenol (3) was prepared following the general procedure J with 2-hydroxy-3-isopropylbenzaldehyde (164 mg, 1 mmol). The final product was isolated by flash column chromatography (DCM:MeOH 95:5, R_{f}=0.5) in 52% (180 mg) yield. ¹H NMR (400 MHZ, MeOH-d₄) δ 8.60-8.39 (m, 2H), 7.67 (td, J=7.7, 1.8 Hz, 2H), 7.37 (d, J=7.8 Hz, 2H), 7.20 (dd, J=7.5, 5.0 Hz, 2H), 7.05 (dd, J=7.7, 1.7 Hz, 1H), 6.87 (dd, J=7.4, 1.7 Hz, 1H), 6.70 (t, J=7.5 Hz, 1H), 3.79 (s, 4H), 3.72 (s, 2H), 3.31 (m, 1H), 1.18 (d, J=7.0 Hz, 6H). ¹³C NMR (101 MHZ, MeOH-d₄) δ 159.28, 155.42, 149.68, 138.52, 136.47, 128.51, 126.61, 124.89, 123.83, 123.45, 120.09, 60.20, 58.31, 27.81, 23.08. HRMS m/z calcd for [M-H]⁻ 346.1925, obsd 346.1924.

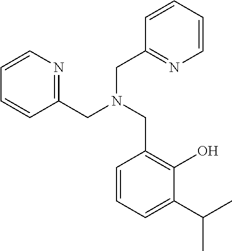 [SMILES: CC(C)c1cccc(CN(Cc2ccccn2)Cc2ccccn2)c1O]

2-((bis(pyridin-2-ylmethyl)amino)methyl)-6-(tert-butyl)phenol (4) was prepared following the general procedure J with 3-(tert-butyl)-2-hydroxybenzaldehyde (178 mg, 1 mmol). The final product was isolated by flash column chromatography (DCM:MeOH 95:5, R_{f}=0.6) in 54% (195 mg) yield. ¹H NMR (400 MHZ, CDCl₃) δ 8.56 (ddd, J=5.0, 1.9, 0.9 Hz, 2H), 7.63 (td, J=7.6, 1.8 Hz, 2H), 7.35 (dt, J=7.8, 1.1 Hz, 2H), 7.23-7.10 (m, 4H), 6.91 (dd, J=7.4, 1.6 Hz, 1H), 6.71 (t, J=7.6 Hz, 1H), 3.86 (s, 4H), 3.82 (s, 2H), 1.46 (s, 10H). ¹³C NMR (101 MHZ, CDCl₃) δ 158.26, 156.66, 149.19, 136.86, 128.18, 126.40, 123.76, 122.94, 122.45, 118.42, 59.53, 58.02, 35.01, 29.75. HRMS m/z calcd for [M-H]⁻ 360.2081, obsd 360.2081.

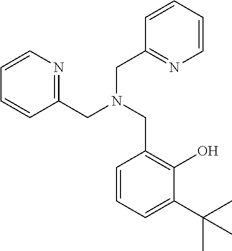 [SMILES: CC(C)(C)c1cccc(CN(Cc2ccccn2)Cc2ccccn2)c1O]

2-((bis(pyridin-2-ylmethyl)amino)methyl)-4-(tert-butyl)phenol (5) was prepared following the general procedure J with 5-(tert-butyl)-2-hydroxybenzaldehyde (178 mg, 1 mmol). The final product was isolated by flash column chromatography (DCM:MeOH 95:5, R_{f}=0.6) in 57% (205 mg) yield. ¹H NMR (400 MHZ, CDCl₃) δ 8.57 (ddd, J=4.9, 1.8, 0.9 Hz, 1H), 7.62 (td, J=7.6, 1.8 Hz, 2H), 7.36 (d, J=7.8 Hz, 2H), 7.23-7.09 (m, 3H), 7.03 (d, J=2.5 Hz, 1H), 6.83 (d, J=8.4 Hz, 1H), 3.88 (s, 4H), 3.78 (s, 2H), 1.27 (s, 9H). ¹³C NMR (101 MHZ, CDCl₃) δ 158.56, 155.27, 149.14, 141.71, 136.94, 127.08, 125.94, 123.47, 122.40, 122.10, 116.04, 77.23, 59.49, 57.71, 34.11, 31.81. HRMS m/z calcd for [M-H]⁻ 360.2081, obsd 360.2080.

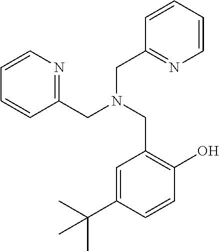 [SMILES: CC(C)(C)c1ccc(O)c(CN(Cc2ccccn2)Cc2ccccn2)c1]

2-((bis(pyridin-2-ylmethyl)amino)methyl)-6-(trimethylsilyl)phenol (6) was prepared following the general procedure J with 2-hydroxy-3-(trimethylsilyl)benzaldehyde (194 mg, 1 mmol). The final product was isolated by flash column chromatography (DCM:MeOH 95:5, R_{f}=0.6) in 61% (230 mg) yield. ¹H NMR (400 MHZ, CDCl₃) δ 10.87 (s, 1H), 8.55 (ddd, J=5.0, 1.8, 0.9 Hz, 2H), 7.62 (td, J=7.7, 1.8 Hz, 2H), 7.34 (dd, J=7.9, 1.1 Hz, 2H), 7.29 (dd, J=7.2, 1.8 Hz, 1H), 7.15 (ddd, J=7.6, 4.9, 1.1 Hz, 2H), 7.06 (dd, J=7.4, 1.7 Hz, 1H), 6.77 (t, J=7.3 Hz, 1H), 3.86 (s, 4H), 3.79 (s, 2H), 0.34 (s, 9H). ¹³C NMR (101 MHZ, CDCl₃) δ 162.78, 158.49, 149.11, 136.85, 134.63, 131.69, 126.29, 123.66, 122.38, 121.82, 118.73, 77.55, 77.23, 76.91, 59.45, 57.61, −0.70. HRMS m/z calcd for [M-H]⁻ 376.1851, obsd 376.1852.

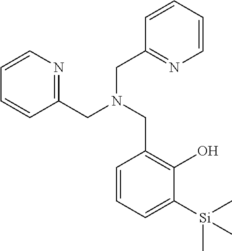 [SMILES: C[Si](C)(C)c1cccc(CN(Cc2ccccn2)Cc2ccccn2)c1O]

2-((bis(pyridin-2-ylmethyl)amino)methyl)-6-(triethylsilyl)phenol (7) was prepared following the general procedure J with 2-hydroxy-3-(triethylsilyl)benzaldehyde (236 mg, 1 mmol). The final product was isolated by flash column chromatography (DCM:MeOH 95:5, R_{f}=0.6) in 57% (222 mg) yield. ¹H NMR (400 MHZ, CDCl₃) δ 10.88 (s, 1H), 8.54 (ddd, J=4.9, 1.8, 0.9 Hz, 2H), 7.62 (td, J=7.7, 1.8 Hz, 2H), 7.34 (dt, J=7.9, 1.1 Hz, 2H), 7.26 (m, 1H), 7.15 (ddd, J=7.4, 4.9, 1.2 Hz, 2H), 7.06 (dd, J=7.4, 1.7 Hz, 1H), 6.76 (t, J=7.3 Hz, 1H), 3.86 (s, 4H), 3.77 (s, 2H), 0.98-0.95 (m, 9H), 0.94-0.89 (m, 6H). ¹³C NMR (101 MHz, CDCl₃) δ 162.98, 158.48, 149.04, 136.83, 135.73, 131.59, 123.56, 123.46, 122.36, 121.66, 118.56, 77.55, 77.23, 76.91, 59.32, 57.58, 7.96, 3.68. HRMS m/z calcd for [M-H]⁻ 418.2320, obsd 418.2324.

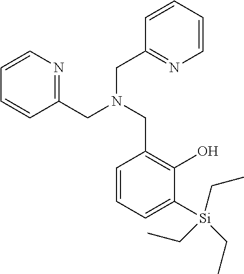 [SMILES: CC[Si](CC)(CC)c1cccc(CN(Cc2ccccn2)Cc2ccccn2)c1O]

2-((bis(pyridin-2-ylmethyl)amino)methyl)-6-(tert-butyldimethylsilyl)phenol (8) was prepared by following the general procedure J with 3-(tert-butyldimethylsilyl)-2-hydroxybenzaldehyde (236 mg, 1 mmol). The final product was isolated by flash column chromatography (DCM:MeOH 95:5, R_{f}=0.7) in 51% (213 mg) yield. ¹H NMR (400 MHZ, CDCl₃) δ 8.54 (ddd, J=4.9, 1.9, 0.9 Hz, 2H), 7.59 (td, J=7.7, 1.8 Hz, 2H), 7.34 (dt, J=7.8, 1.1 Hz, 2H), 7.30 (dd, J=7.3, 1.8 Hz, 1H), 7.13 (ddd, J=7.5, 4.9, 1.2 Hz, 2H), 7.06 (dd, J=7.3, 1.7 Hz, 1H), 6.76 (t, J=7.3 Hz, 1H), 3.88 (s, 4H), 3.78 (s, 2H), 0.96 (s, 9H), 0.37 (s, 6H). ¹³C NMR (101 MHZ, CDCl₃) δ 162.93, 158.38, 148.96, 136.72, 136.10, 131.58, 123.88, 123.46, 122.28, 121.78, 118.33, 77.23, 59.25, 57.64, 27.46, 17.83, −4.35. HRMS m/z calcd for [M-H]. 418.2320, obsd 418.2321.

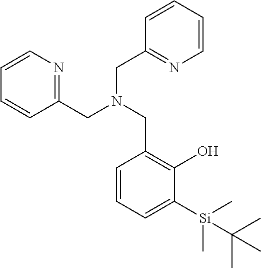 [SMILES: CC(C)(C)[Si](C)(C)c1cccc(CN(Cc2ccccn2)Cc2ccccn2)c1O]

2-((bis(pyridin-2-ylmethyl)amino)methyl)-6-(triisopropylsilyl)phenol (9) was prepared following the general procedure J with 2-hydroxy-3-(triisopropylsilyl)benzaldehyde (278 mg, 1 mmol). The final product was isolated by flash column chromatography (DCM:MeOH 95:5, R_{f}=0.7) in 58% (267 mg) yield. ¹H NMR (400 MHZ, CDCl₃) δ 10.98 (s, 1H), 8.54 (ddd, J=4.8, 1.8, 0.9 Hz, 2H), 7.61 (td, J=7.7, 1.8 Hz, 2H), 7.36 (dt, J=7.9, 1.1 Hz, 2H), 7.31 (dd, J=7.4, 1.7 Hz, 1H), 7.15 (ddd, J=7.6, 4.9, 1.2 Hz, 2H), 7.05 (dd, J=7.3, 1.7 Hz, 1H), 6.75 (t, J=7.3 Hz, 1H), 3.87 (s, 4H), 3.76 (s, 2H), 1.55 (p, J=7.5 Hz, 3H), 1.11 (d, J=7.4 Hz, 18H). ¹³C NMR (101 MHz, CDCl₃) δ 163.28, 158.49, 149.03, 136.83, 136.64, 131.41, 123.46, 122.36, 121.67, 121.65, 118.26, 77.55, 77.23, 76.91, 59.27, 57.71, 19.31, 11.94. HRMS m/z calcd for [M-H]⁻ 460.2790, obsd 460.2799.

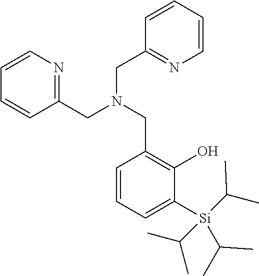 [SMILES: CC(C)[Si](c1cccc(CN(Cc2ccccn2)Cc2ccccn2)c1O)(C(C)C)C(C)C]

2-((bis(pyridin-2-ylmethyl)amino)methyl)-6-(tert-butyldiphenylsilyl)phenol (10) was prepared following the general procedure J with 3-(tert-butyldiphenylsilyl)-2-hydroxybenzaldehyde (360 mg, 1 mmol). The final product was isolated by flash column chromatography (DCM:MeOH 95:5, R_{f}=0.5) in 46% (249 mg) yield. ¹H NMR (400 MHZ, CDCl₃) δ 8.49 (dt, J=4.9, 1.2 Hz, 2H), 7.71-7.50 (m, 6H), 7.42-7.33 (m, 2H), 7.29 (td, J=9.4, 8.5, 4.6 Hz, 7H), 7.17-7.09 (m, 3H), 7.04 (dd, J=7.4, 1.7 Hz, 1H), 6.68 (t, J=7.4 Hz, 1H), 3.89 (s, 4H), 3.83 (s, 2H), 1.27 (s, 9H).¹³C NMR (101 MHz, CDCl₃) δ 163.06, 158.43, 148.93, 138.82, 136.96, 136.64, 136.57, 136.51, 132.44, 128.72, 127.50, 123.42, 122.35, 121.83, 121.63, 118.58, 59.11, 57.69, 30.15, 18.81. HRMS m/z calcd for [M-H]⁻ 542.2633, obsd 542.2636.

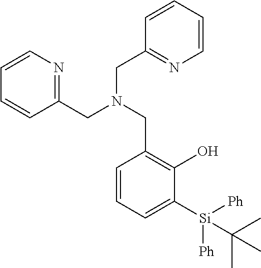 [SMILES: CC(C)(C)[Si](c1ccccc1)(c1ccccc1)c1cccc(CN(Cc2ccccn2)Cc2ccccn2)c1O]

2-((bis(pyridin-2-ylmethyl)amino)methyl)-6-bromophenol (11) was prepared following the general procedure J with 3-bromo-2-hydroxybenzaldehyde (200 mg, 1 mmol). The final product was isolated by flash column chromatography (DCM:MeOH 95:5, R_{f}=0.5) in 52% (200 mg) yield. ¹H NMR (400 MHZ, MeOH-d₄) δ 8.49 (ddd, J=5.0, 1.7, 0.9 Hz, 2H), 7.71 (td, J=7.7, 1.8 Hz, 2H), 7.42-7.30 (m, 3H), 7.24 (ddd, J=7.5, 4.9, 1.1 Hz, 2H), 7.05 (dd, J=7.5, 1.6 Hz, 1H), 6.64 (t, J=7.7 Hz, 1H), 3.82 (s, 4H), 3.77 (s, 2H). ¹³C NMR (101 MHZ, MeOH-d₄) δ 159.17, 155.26, 149.53, 138.63, 133.58, 130.68, 125.96, 124.63, 123.84, 120.96, 111.50, 59.85, 57.73. HRMS m/z calcd for [M-H]⁻ 382.0560, obsd 382.0564.

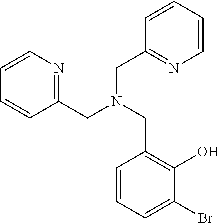 [SMILES: Oc1c(Br)cccc1CN(Cc1ccccn1)Cc1ccccn1]

2-((bis(pyridin-2-ylmethyl)amino)methyl)-6-methoxyphenol (12) was prepared following the general procedure J with 2-hydroxy-3-methoxybenzaldehyde (152 mg, 1 mmol). The final product was isolated by flash column chromatography (DCM:MeOH 95:5, R_{f}=0.5) in 56% (188 mg) yield. ¹H NMR (400 MHZ, MeOH-d₄) δ 8.41 (ddd, J=4.9, 1.7, 0.9 Hz, 2H), 7.63 (td, J=7.7, 1.8 Hz, 2H), 7.40 (dt, J=7.9, 1.1 Hz, 2H), 7.15 (ddd, J=7.5, 5.0, 1.2 Hz, 2H), 6.80 (dd, J=7.7, 1.9 Hz, 1H), 6.76-6.66 (m, 2H), 3.78 (s, 3H), 3.75 (s, 4H), 3.68 (s, 2H). ¹³C NMR (101 MHz, MeOH-d₄) δ 159.67, 149.38, 149.29, 147.29, 138.43, 124.78, 124.46, 123.61, 123.56, 119.92, 112.59, 59.91, 56.53, 56.42. HRMS m/z calcd for [M-H]⁻ 334.1561, obsd 318.1564.

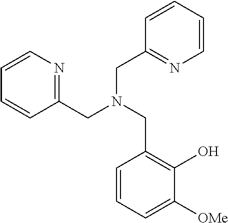 [SMILES: COc1cccc(CN(Cc2ccccn2)Cc2ccccn2)c1O]

General Procedure K: 3-Silylated 2-hydroxybenzaldehyde synthesis

### Scheme S2

An oven-dried round-bottom flask fitted with a stir bar was charged with 2-bromophenol (519 mg, 3 mmol). The flask was capped with a rubber septum, evacuated, and back-filled with nitrogen. This process was repeated an additional three times to ensure an inert atmosphere. Tetrahydrofuran (THF, 20 mL), followed by triethylamine (Et₃N, 626 μL mL, 4.5 mmol) were sequentially added into the flask via syringe. To this mixture, R₃SiCl (4.5 mmol) was added dropwise at 0° C. The mixture was warmed to RT and stirred for 2 h. The reaction mixture was evaporated under reduced pressure, and the residue was filtered through a celite filter and washed with hexane to yield 2-bromophenyl trimethylsilyl ether, which can be used without further purification. The obtained silyl ether was dissolved in dry THF (20 mL) under nitrogen. After the mixture was cooled to −78° C., a solution of n-BuLi (2.6 M in hexane, 1.3 mL, 3 mmol) was added dropwise into the mixture. The flask was warmed to RT and stirred for 2 h. The reaction was quenched with a saturated NH₄Cl solution (15 mL) and the mixture was extracted three times with ethyl acetate. The combined organic extracts were dried over anhydrous Na₂SO₄, filtered, and concentrated under reduced pressure. The residue was purified by flash column chromatography on silica gel (hexane/ethyl acetate [EtOAc]) to proceed to the next step.⁵

To a solution of 2-silylated phenol (1.0 eq.) and MgCl₂ (2.0 eq.) in acetonitrile (10 mL) was added Et₃N (3.75 eq.) at RT and stirred for 15 min. Paraformaldehyde (6.75 eq.) was added to the mixture, and the resulting mixture was stirred under reflux until complete consumption of the starting material (monitored by TLC; ˜3 hr). The reaction mixture was poured into 1 M aqueous HCl. After removing the volatiles, the residue was further dissolved in EtOAc and filtered through a pad of Celite. The filtrate was extracted with EtOAc (3×20 mL) and was washed with brine (1×20 mL). The combined organic layer was dried over anhydrous Na₂SO₄, filtered, and concentrated. The crude mixture was purified by flash column chromatography on silica gel to yield the desired aldehyde.⁶

2-hydroxy-3-(trimethylsilyl)benzaldehyde (S6) was prepared following general procedure K using Mes SiCl (489 mg, 4.5 mmol) as the silyl source. The final product was isolated by flash column chromatography (Hexane:EtOAc 90:10, R_{f}=0.8) in 72% overall yield (419 mg). ¹H NMR (400 MHZ, CDCl₃) δ 11.31 (s, 1H), 9.88 (s, 1H), 7.62 (dd, J=7.1, 1.8 Hz, 1H), 7.54 (dd, J=7.6, 1.8 Hz, 1H), 7.01 (t, J=7.4 Hz, 1H), 0.33 (s, 9H). ¹³C NMR (101 MHZ, CDCl₃) δ 197.05, 166.53, 142.84, 135.09, 128.94, 119.80, 119.75, 77.23, −1.07.

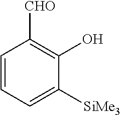 [SMILES: C[Si](C)(C)c1cccc(C=O)c1O]

2-hydroxy-3-(triethylsilyl)benzaldehyde (S7) was prepared following general procedure K using Et₃SiCl (678 mg, 4.5 mmol) as the silyl source. The final product was isolated by flash column chromatography (Hexane:EtOAc 90:10, R_{f}=0.8) in 63% overall yield. ¹H NMR (400 MHZ, DMSO-d₆) δ 11.35 (s, 1H), 9.97 (s, 1H), 7.79 (dd, J=7.7, 1.8 Hz, 1H), 7.61 (dd, J=7.2, 1.8 Hz, 1H), 7.09 (t, J=7.4 Hz, 1H), 0.94-0.86 (m, 9H), 0.86-0.77 (m, 6H). ¹³C NMR (101 MHz, DMSO-d₆) δ 198.12, 165.38, 143.31, 135.35, 124.39, 119.93, 119.72, 7.36, 2.72. HRMS m/z calcd for [M-H]⁻ 235.1160, obsd 235.1162.

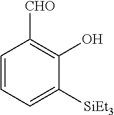

3-(tert-butyldimethylsilyl)-2-hydroxybenzaldehyde (S8) was prepared following general procedure K using MeztBuSiCl (678 mg, 4.5 mmol) as the silyl source. The final product was isolated by flash column chromatography (Hexane:EtOAc 90:10, R_{f}=0.9) in 58% overall yield. ¹H NMR (400 MHZ, CDCl₃) δ 11.38 (s, 1H), 9.87 (s, 1H), 7.63 (dt, J=7.2, 1.4 Hz, 1H), 7.54 (dd, J=7.6, 1.8 Hz, 1H), 7.01 (t, J=7.4 Hz, 1H), 0.92 (s, 9H), 0.33 (s, 6H). ¹³C NMR (101 MHz, CDCl₃) δ 197.05, 166.80, 144.40, 135.29, 126.75, 119.91, 119.56, 27.15, 17.77, −4.70. HRMS m/z calcd for [M-H]⁻ 235.1160, obsd 235.1168.

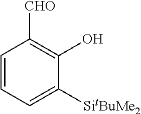

2-hydroxy-3-(triisopropylsilyl)benzaldehyde (S9) was prepared following general procedure K using iPr₃SiCl (868 mg, 4.5 mmol) as the silyl source. The final product was isolated by flash column chromatography (Hexane:EtOAc 90:10, R_{f}=0.9) in 62% overall yield. ¹H NMR (400 MHZ, CDCl₃) δ 11.44 (s, 1H), 9.87 (s, 1H), 7.66 (dd, J=7.2, 1.8 Hz, 1H), 7.54 (dd, J=7.6, 1.8 Hz, 1H), 7.02 (t, J=7.4 Hz, 1H), 1.54 (td, J=15.0, 14.2, 6.7 Hz, 3H), 1.10 (d, J=7.7 Hz, 18 H). ¹³C NMR (101 MHz, CDCl₃) δ 197.08, 167.17, 144.86, 135.08, 124.65, 119.96, 119.60, 19.02, 11.73. HRMS m/z calcd for [M-H]⁻ 277.1629, obsd 277.1629.

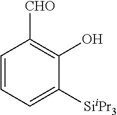

3-(tert-butyldiphenylsilyl)-2-hydroxybenzaldehyde (S10) was prepared following general procedure K using Ph₂tBuSiCl (1.237 g, 4.5 mmol) as the silyl source. The final product was isolated by flash column chromatography (Hexane:EtOAc 90:20, R_{f}=0.7) in 54% overall yield. ¹H NMR (400 MHZ, CDCl₃) δ 11.63 (s, 1H), 9.93 (s, 1H), 7.62 (dd, J=7.6, 1.8 Hz, 1H), 7.59-7.53 (m, 4H), 7.46-7.34 (m, 8H), 6.95 (t, J=7.4 Hz, 1H), 1.23 (s, 9H). ¹³C NMR (101 MHZ, CDCl₃) δ 163.06, 158.43, 148.93, 138.82, 136.96, 136.64, 136.57, 136.51, 132.44, 128.72, 127.50, 123.42, 122.35, 121.83, 121.63, 118.58, 59.11, 57.69, 30.15, 18.81. HRMS m/z calcd for [M-H]⁻ 359.1473, obsd 359.1476.

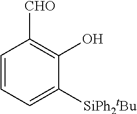

General Procedure L. Zn:9 complexation

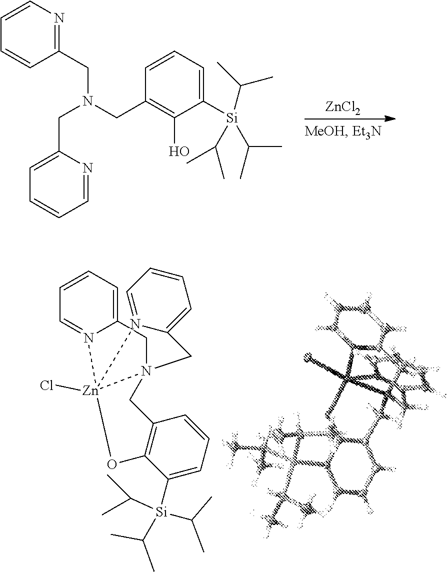

An oven-dried 25 mL round-bottom flask fitted with a stir bar was charged with 9 (138 mg, 0.3 mmol). The flask was capped with a rubber septum, evacuated, and back-filled with nitrogen. This process was repeated for additional three times to achieve an inert atmosphere. Methanol (5 mL) followed by Et₃N (42 μL, 0.3 mmol) and anhydrous ZnCl₂ (41 mg, 0.3 mmol) were added sequentially into the flask. The mixture was stirred at RT for 48 h. The resultant white turbid solution was filtered and decanted into a 20-mL vial. The vial with filtered solution was submerged in an Erlenmeyer flask containing a small amount of pentane (10 mL). The flask was sealed with parafilm, and the Zn: 9 was crystalized from the saturated methanol solution.

X-ray Crystallography: A crystal mounted on a diffractometer collected data at 100 K. The intensities of the reflections were collected by means of a Bruker APEX II CCD diffractometer (Moka radiation, λ=0.71073 Å) equipped with an Oxford Cryosystems nitrogen flow apparatus. The collection method involved 0.5° scans in ω at 28° in 2θ. Data integration down to 0.77 Å resolution was carried out using SAINT V8.37A¹ with reflection spot size optimization. Absorption corrections were made with the program SADABS.^{1,2} The structure was solved using the Intrinsic Phasing methods and refined by least-squares methods against F² using SHELXT-2014³ and SHELXL-2014⁴ with the OLEX 2 interface.⁵ Non-hydrogen atoms were refined anisotropically, and hydrogen atoms were allowed to ride on the respective atoms. Crystal data as well as data collection and refinement details are summarized in Table 5, geometric parameters are shown in Table 6, and hydrogen-bond parameters are listed in Table 7. The Ortep plots were produced with the SHELXL-2014 program, and the three-dimensional supramolecular architecture drawing was produced with Accelrys DS Visualizer 2.06.⁶

### References for Materials Section

1. S. Akine, TitrationFit, program for analyses of host-guest complexation, Kanazawa University, Kanazawa, Japan, 2013.
2. Clinical and Laboratory Standards Institute. M100 Performance Standards for Antimicrobial Susceptibility Testing (Clinical and Laboratory Standards Institute, ed. 27, 2017).
3. Clinical and Laboratory Standards Institute. Methods for Dilution Antimicrobial Susceptibility Tests for Bacteria that Grow Aerobically; Approved Standard-Eighth Edition (CLSI document M07-A8). Clinical and Laboratory Standards Institute, Wayne, PA (2009). 4. D. Song, J. M. Lim, S. Cho, S. J. Park, D. Kang, S. G. Rhee, Y. You, W. Nam, Chem. Commun. 2012, 48, 5449.
5 T. Ikawa, T. Nishiyama, T. Nosaki, A. Takagi, S. Akai, Org. Lett. 2011, 13, 1730-1733.
6. T. Yoshimura, K. Tomohara, T. Kawabata, J. Am. Chem. Soc. 2013, 135, 7102-7105.
7. Bruker AXS APEX3, Bruker AXS, Madison, Wisconsin, 2015.
8. L. Krause, R. Herbst-Irmer, G. M. Sheldrick, D. Stalke, J. Appl. Crystallogr. 2015, 48, 3-10.
9. G. M. Sheldrick, Acta Cryst. 2015. A71, 3-8.
10. G. M. Sheldrick, Acta Cryst. 2015, C71, 3-8.
11. O. V. Dolomanov, L. J. Bourhis, R. J. Gildea, J. A. K. Howard and H. Puschmann J. Appl. Cryst. 2009, 42, 339-341.
12. Accelrys DS Visualizer v2.0.1, Accelrys Software. Inc., 2007.

TABLE 5
X-ray Experimental details

Compound 9

Crystal data

| Chemical formula | C₂₉H₄₂ClN₃O₂SiZn | |
|---|---|---|
| Mᵣ | 593.56 | |
| Crystal system, | Monoclinic, P2₁ | |
| space group | | |
| Temperature (K) | 100 | |
| a, b, c (Å) | 9.2455 (8), 13.7881 (12), | |
| | 11.9354 (10) | |
| (°) | 103.5091 | (14) |
| V (Å³) | 1479.4 | (2) |

| | |
|---|---|
| Z | 2 |
| Radiation type | Mo K |
| (mm⁻¹) | 0.99 |
| Crystal size (mm) | 0.18 × 0.14 × 0.10 |

Data collection

| | |
|---|---|
| Diffractometer | Bruker D8 goniometer with |
| | CCD area detector |
| Absorption | Multi-scan |
| correction | SADABS |
| Tₘᵢₙ, Tₘₐₓ | 0.625, 0.746 |
| No. of measured, | 36015, 6702, 6524 |
| independent and | |
| observed [I > 2 (I)] | |
| reflections | |
| Rᵢₙₜ | 0.025 |
| (sin/)ₘₐₓ (Å⁻¹) | 0.650 |

Refinement

| R[F² > 2 (F²)], | 0.020, 0.054, 1.06 | |
|---|---|---|
| wR(F²), S | | |
| No. of reflections | 6702 | |
| No. of parameters | 342 | |
| No. of restraints | 1 | |
| H-atom treatment | H-atom parameters constrained | |
| rₘₐₓ, rₘᵢₙ (e Å⁻³) | 0.42, −0.24 | |
| Absolute structure | Flack x determined using 3007 quotients | |
| | [(I+) − (I−)]/[(I+) + (I−)] (Parsons, Flack | |
| | and Wagner, Acta Cryst. B69 (2013) 249-259). | |
| Absolute structure | −0.012 | (2) |
| parameter | | |

Computer programs: SAINT 8.37A (Bruker-AXS, 2015), SHELXT2014 (Sheldrick, 2015), SHELXL2014 (Sheldrick, 2015), Bruker SHELXTL (Sheldrick, 2015).

TABLE 6
Geometric parameters (Å, °)

| Zn1—O1 | 1.9403 | (15) | C14—H14B | 0.9900 | |
|---|---|---|---|---|---|
| Zn1—N2 | 2.1024 | (19) | C15—C16 | 1.390 | (3) |
| Zn1—N3 | 2.1078 | (18) | C16—C17 | 1.381 | (4) |
| Zn1—N1 | 2.2667 | (18) | C16—H16 | 0.9500 | |
| Zn1—C11 | 2.3231 | (6) | C17—C18 | 1.382 | (4) |
| Si1—C2 | 1.888 | (2) | C17—H17 | 0.9500 | |
| Si1—C23 | 1.904 | (2) | C18—C19 | 1.387 | (3) |
| Si1—C20 | 1.906 | (2) | C18—H18 | 0.9500 | |
| Si1—C26 | 1.908 | (2) | C19—H19 | 0.9500 | |
| O1—C1 | 1.328 | (3) | C20—C21 | 1.533 | (4) |
| N1—C8 | 1.465 | (3) | C20—C22 | 1.545 | (3) |
| N1—C14 | 1.471 | (3) | C20—H20 | 1.0000 | |
| N1—C7 | 1.486 | (3) | C21—H21A | 0.9800 | |
| N2—C9 | 1.346 | (3) | C21—H21B | 0.9800 | |
| N2—C13 | 1.349 | (3) | C21—H21C | 0.9800 | |
| N3—C19 | 1.341 | (3) | C22—H22A | 0.9800 | |
| N3—C15 | 1.344 | (3) | C22—H22B | 0.9800 | |
| C1—C6 | 1.419 | (3) | C22—H22C | 0.9800 | |
| C1—C2 | 1.426 | (3) | C23—C24 | 1.537 | (3) |
| C2—C3 | 1.400 | (3) | C23—C25 | 1.539 | (3) |
| C3—C4 | 1.392 | (4) | C23—H23 | 1.0000 | |
| C3—H3 | 0.9500 | | C24—H24A | 0.9800 | |
| C4—C5 | 1.387 | (4) | C24—H24B | 0.9800 | |
| C4—H4 | 0.9500 | | C24—H24C | 0.9800 | |
| C5—C6 | 1.405 | (3) | C25—H25A | 0.9800 | |
| C5—H5 | 0.9500 | | C25—H25B | 0.9800 | |
| C6—C7 | 1.505 | (3) | C25—H25C | 0.9800 | |
| C7—H7A | 0.9900 | | C26—C28 | 1.538 | (3) |
| C7—H7B | 0.9900 | | C26—C27 | 1.542 | (4) |
| C8—C9 | 1.516 | (3) | C26—H26 | 1.0000 | |
| C8—H8A | 0.9900 | | C27—H27A | 0.9800 | |
| C8—H8B | 0.9900 | | C27—H27B | 0.9800 | |
| C9—C10 | 1.388 | (3) | C27—H27C | 0.9800 | |
| C10—C11 | 1.388 | (3) | C28—H28A | 0.9800 | |
| C10—H10 | 0.9500 | | C28—H28B | 0.9800 | |
| C11—C12 | 1.384 | (4) | C28—H28C | 0.9800 | |
| C11—H11 | 0.9500 | | O1S—C1S | 1.411 | (4) |
| C12—C13 | 1.382 | (3) | O1S—H1S | 0.8400 | |
| C12—H12 | 0.9500 | | C1S—H1SA | 0.9800 | |
| C13—H13 | 0.9500 | | C1S—H1SB | 0.9800 | |
| C14—C15 | 1.506 | (3) | C1S—H1SC | 0.9800 | |
| C14—H14A | 0.9900 | | | | |
| O1—Zn1—N2 | 123.11 | (7) | C15—C14—H14B | 109.3 | |
| O1—Zn1—N3 | 119.93 | (7) | H14A—C14—H14B | 107.9 | |
| N2—Zn1—N3 | 110.38 | (7) | N3—C15—C16 | 121.4 | (2) |
| O1—Zn1—N1 | 89.15 | (6) | N3—C15—C14 | 116.47 | (18) |
| N2—Zn1—N1 | 77.05 | (7) | C16—C15—C14 | 122.0 | (2) |
| N3—Zn1—N1 | 77.38 | (7) | C17—C16—C15 | 119.4 | (2) |
| O1—Zn1—Cl1 | 97.90 | (5) | C17—C16—H16 | 120.3 | |
| N2—Zn1—Cl1 | 100.90 | (5) | C15—C16—H16 | 120.3 | |
| N3—Zn1—Cl1 | 96.85 | (5) | C16—C17—C18 | 119.2 | (2) |
| N1—Zn1—Cl1 | 172.53 | (5) | C16—C17—H17 | 120.4 | |
| C2—Si1—C23 | 106.83 | (10) | C18—C17—H17 | 120.4 | |
| C2—Si1—C20 | 110.58 | (10) | C17—C18—C19 | 118.4 | (2) |
| C23—Si1—C20 | 108.25 | (12) | C17—C18—H18 | 120.8 | |
| C2—Si1—C26 | 112.79 | (11) | C19—C18—H18 | 120.8 | |
| C23—Si1—C26 | 110.61 | (12) | N3—C19—C18 | 122.6 | (2) |
| C20—Si1—C26 | 107.71 | (10) | N3—C19—H19 | 118.7 | |
| C1—O1—Zn1 | 129.22 | (13) | C18—C19—H19 | 118.7 | |
| C8—N1—C14 | 111.39 | (18) | C21—C20—C22 | 109.1 | (2) |
| C8—N1—C7 | 113.56 | (17) | C21—C20—Si1 | 114.30 | (18) |
| C14—N1—C7 | 109.94 | (16) | C22—C20—Si1 | 114.84 | (17) |
| C8—N1—Zn1 | 105.67 | (12) | C21—C20—H20 | 105.9 | |
| C14—N1—Zn1 | 106.37 | (13) | C22—C20—H20 | 105.9 | |
| C7—N1—Zn1 | 109.57 | (13) | Si1—C20—H20 | 105.9 | |
| C9—N2—C13 | 118.36 | (19) | C20—C21—H21A | 109.5 | |
| C9—N2—Zn1 | 116.78 | (14) | C20—C21—H21B | 109.5 | |
| C13—N2—Zn1 | 124.71 | (15) | H21A—C21—H21B | 109.5 | |
| C19—N3—C15 | 118.89 | (19) | C20—C21—H21C | 109.5 | |
| C19—N3—Zn1 | 123.87 | (15) | H21A—C21—H21C | 109.5 | |
| C15—N3—Zn1 | 117.19 | (15) | H21B—C21—H21C | 109.5 | |
| O1—C1—C6 | 121.28 | (19) | C20—C22—H22A | 109.5 | |
| O1—C1—C2 | 119.01 | (19) | C20—C22—H22B | 109.5 | |
| C6—C1—C2 | 119.7 | (2) | H22A—C22—H22B | 109.5 | |
| C3—C2—C1 | 118.3 | (2) | C20—C22—H22C | 109.5 | |
| C3—C2—Si1 | 123.67 | (17) | H22A—C22—H22C | 109.5 | |
| C1—C2—Si1 | 118.02 | (16) | H22B—C22—H22C | 109.5 | |
| C4—C3—C2 | 122.2 | (2) | C24—C23—C25 | 110.4 | (2) |
| C4—C3—H3 | 118.9 | | C24—C23—Si1 | 113.59 | (17) |
| C2—C3—H3 | 118.9 | | C25—C23—Si1 | 113.72 | (17) |
| C5—C4—C3 | 119.3 | (2) | C24—C23—H23 | 106.2 | |
| C5—C4—H4 | 120.4 | | C25—C23—H23 | 106.2 | |
| C3—C4—H4 | 120.4 | | Si1—C23—H23 | 106.2 | |
| C4—C5—C6 | 121.1 | (2) | C23—C24—H24A | 109.5 | |
| C4—C5—H5 | 119.5 | | C23—C24—H24B | 109.5 | |
| C6—C5—H5 | 119.5 | | H24A—C24—H24B | 109.5 | |
| C5—C6—C1 | 119.4 | (2) | C23—C24—H24C | 109.5 | |
| C5—C6—C7 | 119.4 | (2) | H24A—C24—H24C | 109.5 | |
| C1—C6—C7 | 121.17 | (19) | H24B—C24—H24C | 109.5 | |
| N1—C7—C6 | 113.47 | (17) | C23—C25—H25A | 109.5 | |
| N1—C7—H7A | 108.9 | | C23—C25—H25B | 109.5 | |
| C6—C7—H7A | 108.9 | | H25A—C25—H25B | 109.5 | |
| N1—C7—H7B | 108.9 | | C23—C25—H25C | 109.5 | |
| C6—C7—H7B | 108.9 | | H25A—C25—H25C | 109.5 | |
| H7A—C7—H7B | 107.7 | | H25B—C25—H25C | 109.5 | |
| N1—C8—C9 | 110.26 | (18) | C28—C26—C27 | 110.0 | (2) |
| N1—C8—H8A | 109.6 | | C28—C26—Si1 | 117.14 | (18) |
| C9—C8—H8A | 109.6 | | C27—C26—Si1 | 113.19 | (18) |
| N1—C8—H8B | 109.6 | | C28—C26—H26 | 105.1 | |
| C9—C8—H8B | 109.6 | | C27—C26—H26 | 105.1 | |
| H8A—C8—H8B | 108.1 | | Si1—C26—H26 | 105.1 | |
| N2—C9—C10 | 122.5 | (2) | C26—C27—H27A | 109.5 | |
| N2—C9—C8 | 116.20 | (19) | C26—C27—H27B | 109.5 | |
| C10—C9—C8 | 121.3 | (2) | H27A—C27—H27B | 109.5 | |
| C9—C10—C11 | 118.4 | (2) | C26—C27—H27C | 109.5 | |
| C9—C10—H10 | 120.8 | | H27A—C27—H27C | 109.5 | |
| C11—C10—H10 | 120.8 | | H27B—C27—H27C | 109.5 | |
| C12—C11—C10 | 119.5 | (2) | C26—C28—H28A | 109.5 | |
| C12—C11—H11 | 120.3 | | C26—C28—H28B | 109.5 | |
| C10—C11—H11 | 120.3 | | H28A—C28—H28B | 109.5 | |
| C13—C12—C11 | 118.8 | (2) | C26—C28—H28C | 109.5 | |
| C13—C12—H12 | 120.6 | | H28A—C28—H28C | 109.5 | |
| C11—C12—H12 | 120.6 | | H28B—C28—H28C | 109.5 | |
| N2—C13—C12 | 122.4 | (2) | C1S—O1S—H1S | 109.5 | |
| N2—C13—H13 | 118.8 | | O1S—C1S—H1SA | 109.5 | |
| C12—C13—H13 | 118.8 | | O1S—C1S—H1SB | 109.5 | |
| N1—C14—C15 | 111.83 | (18) | H1SA—C1S—H1SB | 109.5 | |
| N1—C14—H14A | 109.3 | | O1S—C1S—H1SC | 109.5 | |
| C15—C14—H14A | 109.3 | | H1SA—C1S—H1SC | 109.5 | |
| N1—C14—H14B | 109.3 | | H1SB—C1S—H1SC | 109.5 | |
| Zn1—O1—C1—C6 | −40.5 | (3) | C13—N2—C9—C10 | −0.2 | (3) |
| Zn1—O1—C1—C2 | 141.31 | (16) | Zn1—N2—C9—C10 | 175.57 | (17) |
| O1—C1—C2—C3 | 175.20 | (19) | C13—N2—C9—C8 | 177.45 | (19) |
| C6—C1—C2—C3 | −3.0 | (3) | Zn1—N2—C9—C8 | −6.7 | (2) |
| O1—C1—C2—Si1 | −3.0 | (3) | N1—C8—C9—N2 | 33.2 | (3) |
| C6—C1—C2—Si1 | 178.74 | (15) | N1—C8—C9—C10 | −149.1 | (2) |
| C23—Si1—C2—C3 | −107.1 | (2) | N2—C9—C10—C11 | 0.4 | (3) |
| C20—Si1—C2—C3 | 135.3 | (2) | C8—C9—C10—C11 | −177.2 | (2) |
| C26—Si1—C2—C3 | 14.7 | (2) | C9—C10—C11—C12 | −0.2 | (4) |
| C23—Si1—C2—C1 | 71.07 | (18) | C10—C11—C12—C13 | −0.1 | (4) |
| C20—Si1—C2—C1 | −46.5 | (2) | C9—N2—C13—C12 | −0.1 | (3) |
| C26—Si1—C2—C1 | −167.19 | (17) | Zn1—N2—C13—C12 | −175.54 | (18) |
| C1—C2—C3—C4 | 2.4 | (3) | C11—C12—C13—N2 | 0.3 | (4) |
| Si1—C2—C3—C4 | −179.47 | (18) | C8—N1—C14—C15 | −149.77 | (18) |
| C2—C3—C4—C5 | −0.3 | (4) | C7—N1—C14—C15 | 83.5 | (2) |
| C3—C4—C5—C6 | −1.2 | (4) | Zn1—N1—C14—C15 | −35.1 | (2) |
| C4—C5—C6—C1 | 0.5 | (3) | C19—N3—C15—C16 | −0.1 | (3) |
| C4—C5—C6—C7 | −177.5 | (2) | Zn1—N3—C15—C16 | 177.53 | (18) |
| O1—C1—C6—C5 | −176.56 | (19) | C19—N3—C15—C14 | 175.89 | (19) |
| C2—C1—C6—C5 | 1.6 | (3) | Zn1—N3—C15—C14 | −6.5 | (2) |
| O1—C1—C6—C7 | 1.5 | (3) | N1—C14—C15—N3 | 29.7 | (3) |
| C2—C1—C6—C7 | 179.6 | (2) | N1—C14—C15—C16 | −154.3 | (2) |
| C8—N1—C7—C6 | 55.8 | (2) | N3—C15—C16—C17 | 0.0 | (4) |
| C14—N1—C7—C6 | −178.62 | (19) | C14—C15—C16—C17 | −175.8 | (2) |
| Zn1—N1—C7—C6 | −62.1 | (2) | C15—C16—C17—C18 | −0.4 | (4) |
| C5—C6—C7—N1 | −127.5 | (2) | C16—C17—C18—C19 | 0.8 | (4) |
| C1—C6—C7—N1 | 54.5 | (3) | C15—N3—C19—C18 | 0.6 | (3) |
| C14—N1—C8—C9 | 75.4 | (2) | Zn1—N3—C19—C18 | −176.90 | (18) |
| C7—N1—C8—C9 | −159.82 | (17) | C17—C18—C19—N3 | −0.9 | (4) |

| | | |
|---|---|---|
| Zn1—N1—C8—C9 | −39.70 | (19) |

TABLE 7
Hydrogen-bond parameters

| | D—H | H . . . A | D . . . A | D—H . . . A |
|---|---|---|---|---|
| D—H . . . A | (Å) | (Å) | (Å) | (°) |
| O1S—H1S . . . Cl1ⁱ | 0.84 | 2.32 | 3.129 (2) | 160.9 |

### References for Materials and Methods

1. S. Akine, TitrationFit, program for analyses of host-guest complexation, Kanazawa University, Kanazawa, Japan, 2013.
2. Clinical and Laboratory Standards Institute. M100 Performance Standards for Antimicrobial Susceptibility Testing (Clinical and Laboratory Standards Institute, ed. 27, 2017).
3. Clinical and Laboratory Standards Institute. Methods for Dilution Antimicrobial Susceptibility Tests for Bacteria that Grow Aerobically; Approved Standard-Eighth Edition (CLSI document M07-A8). Clinical and Laboratory Standards Institute, Wayne, PA (2009).
4. D. Song, J. M. Lim, S. Cho, S. J. Park, D. Kang, S. G. Rhee, Y. You, W. Nam, Chem. Commun. 2012, 48, 5449.
5. T. Ikawa, T. Nishiyama, T. Nosaki, A. Takagi, S. Akai, Org. Lett. 2011, 13, 1730-1733.
6. T. Yoshimura, K. Tomohara, T. Kawabata, J. Am. Chem. Soc. 2013, 135, 7102-7105.
7. Bruker AXS APEX3, Bruker AXS, Madison, Wisconsin, 2015.
8. L. Krause, R. Herbst-Irmer, G. M. Sheldrick, D. Stalke, J. Appl. Crystallogr. 2015, 48, 3-10.
9. G. M. Sheldrick, Acta Cryst. 2015. A71, 3-8.
10. G. M. Sheldrick, Acta Cryst. 2015, C71, 3-8.
11. O. V. Dolomanov, L. J. Bourhis, R. J. Gildea, J. A. K. Howard and H. Puschmann J. Appl. Cryst. 2009, 42, 339-341. 12.Accelrys DS Visualizer v2.0.1, Accelrys Software. Inc., 2007.

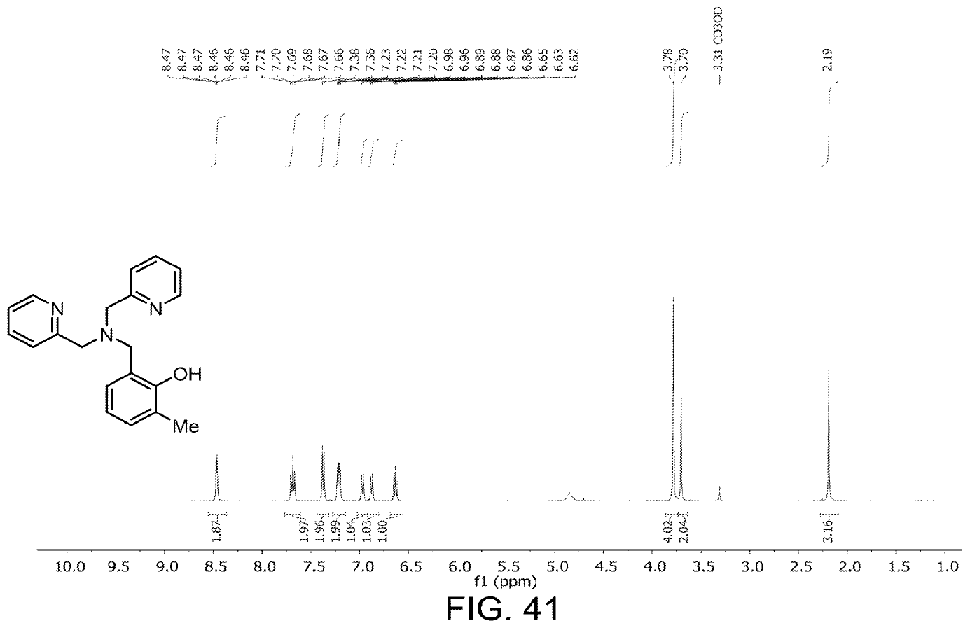

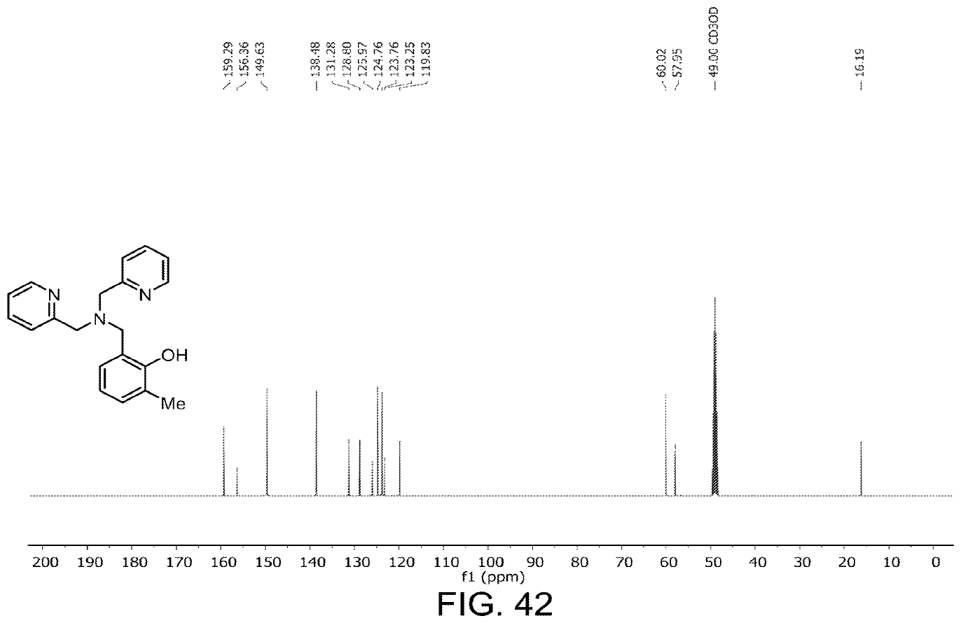

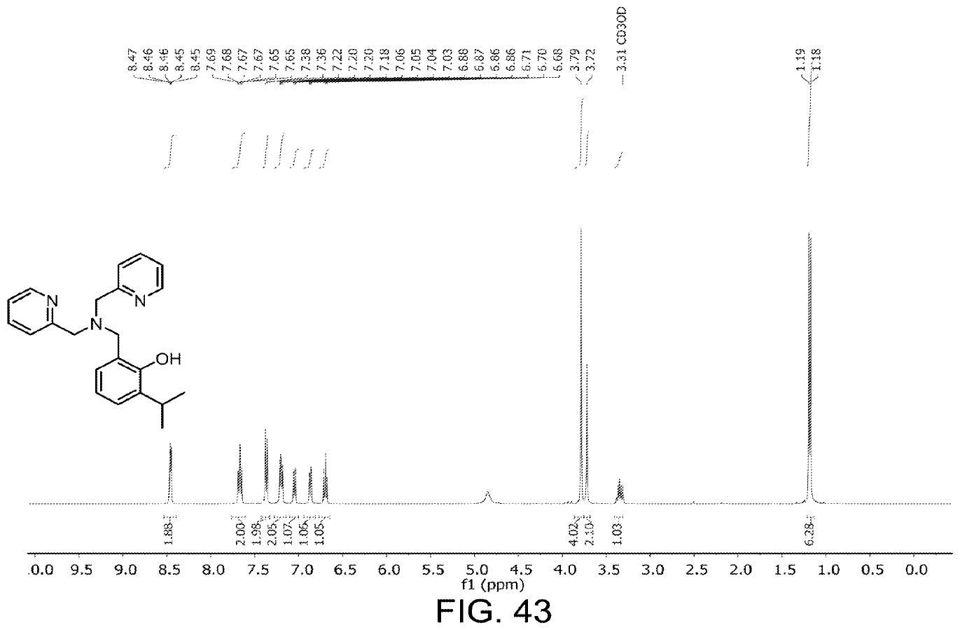

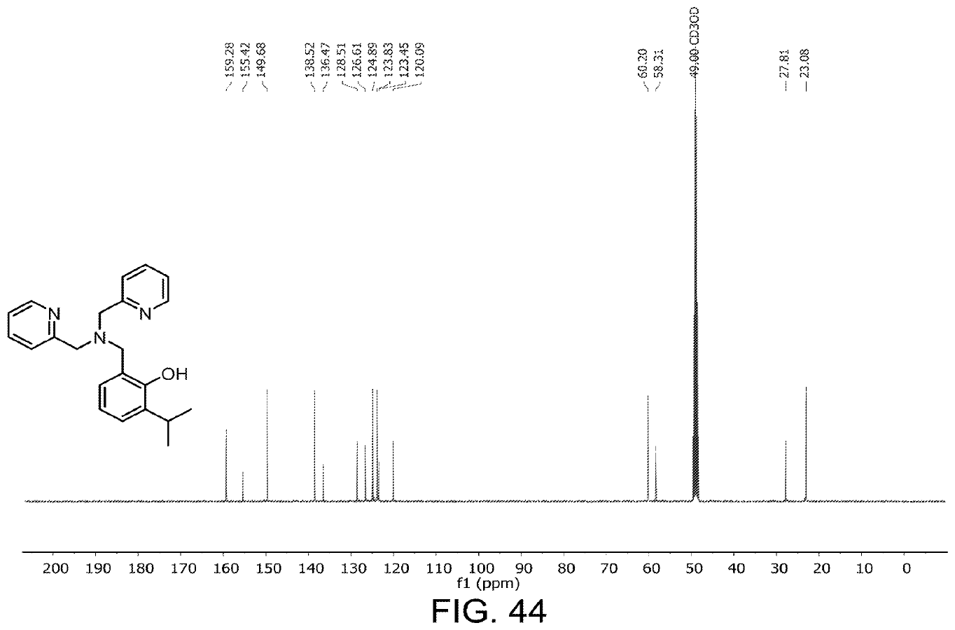

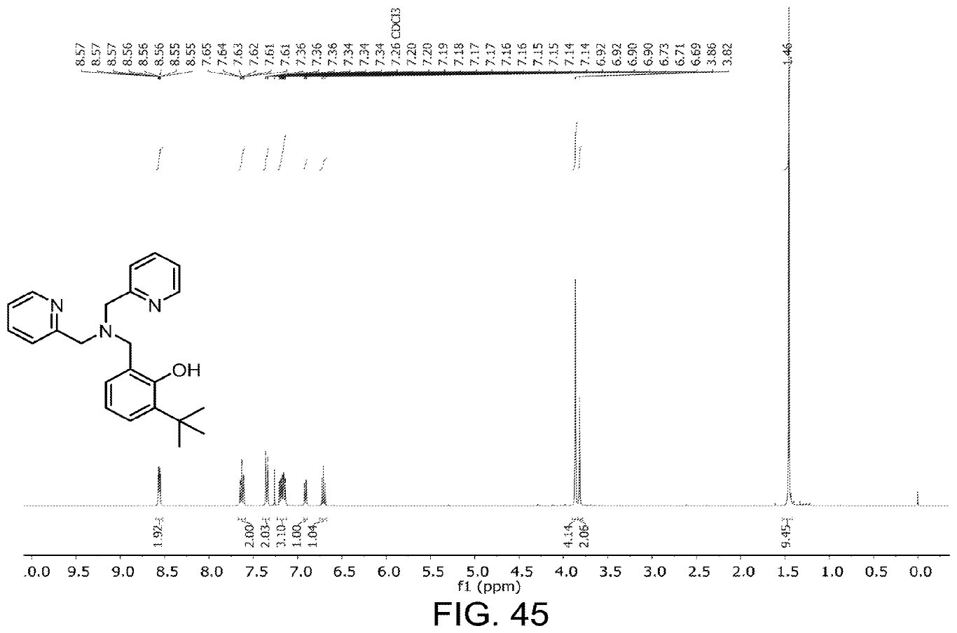

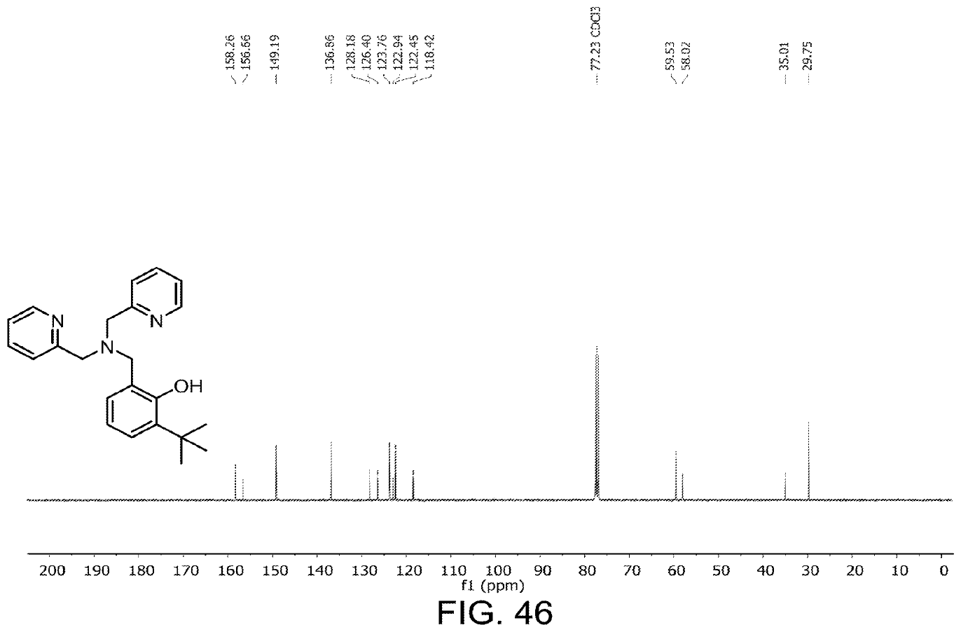

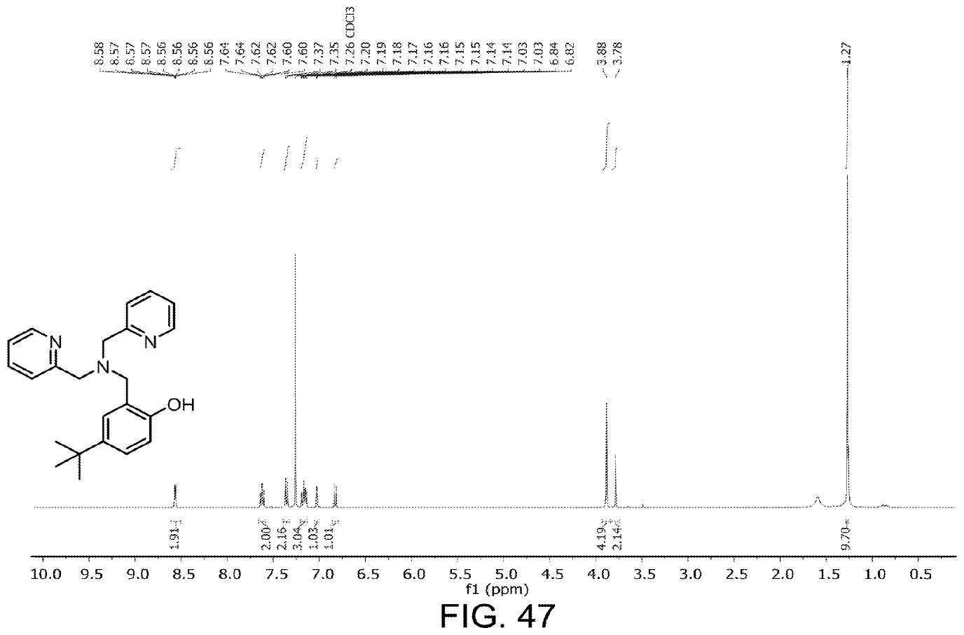

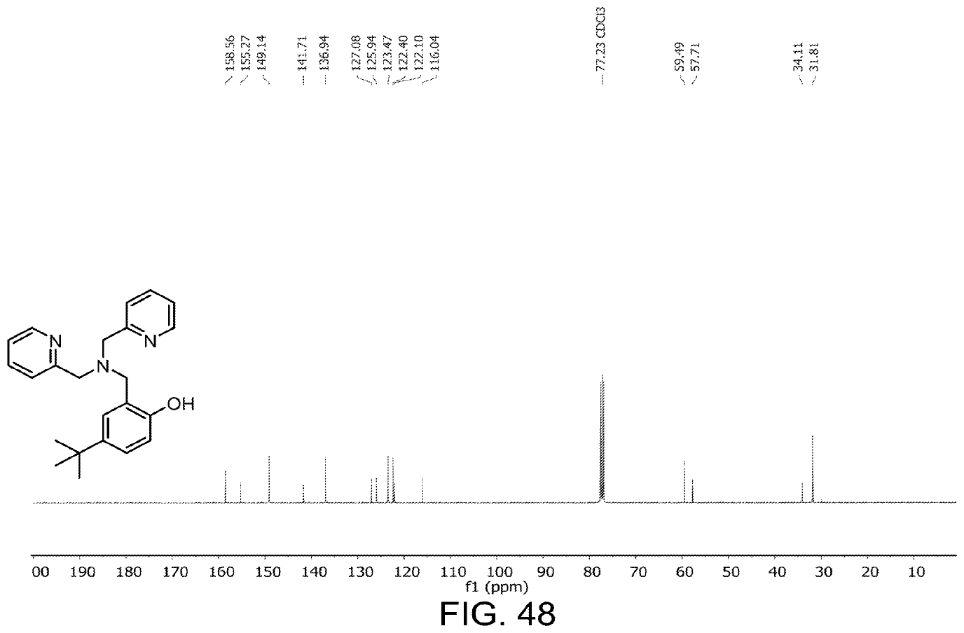

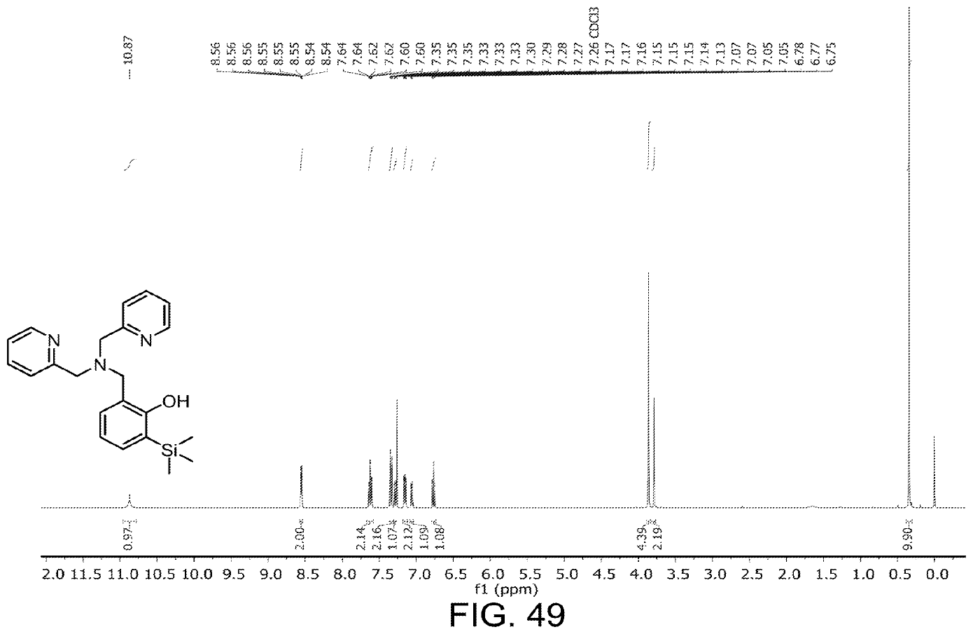

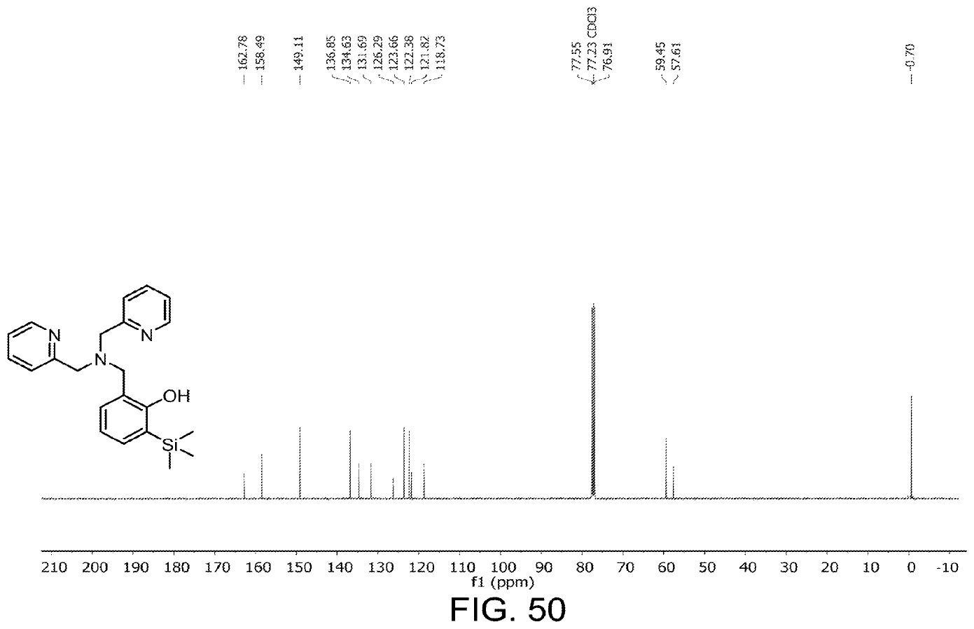

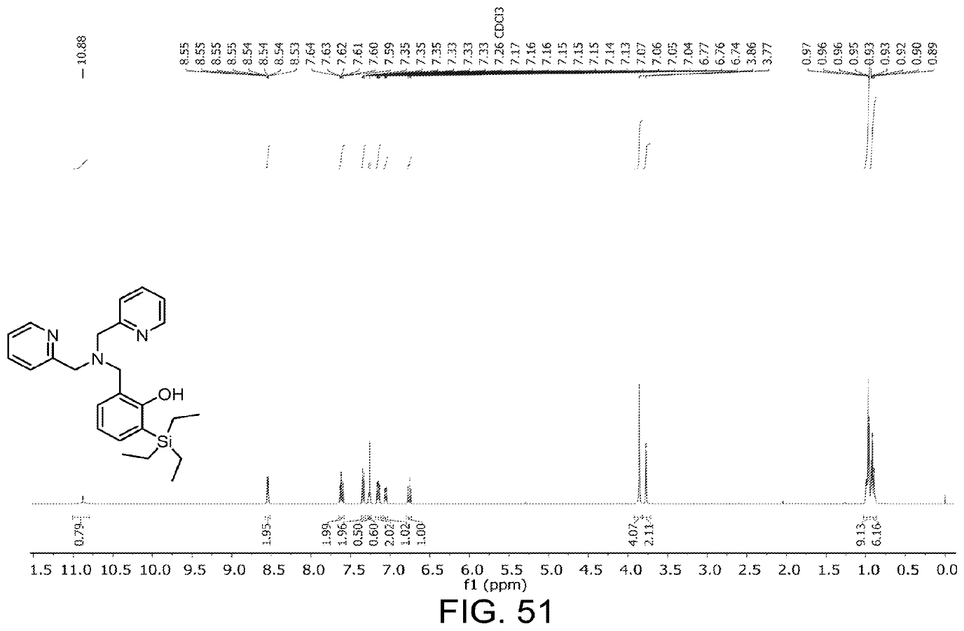

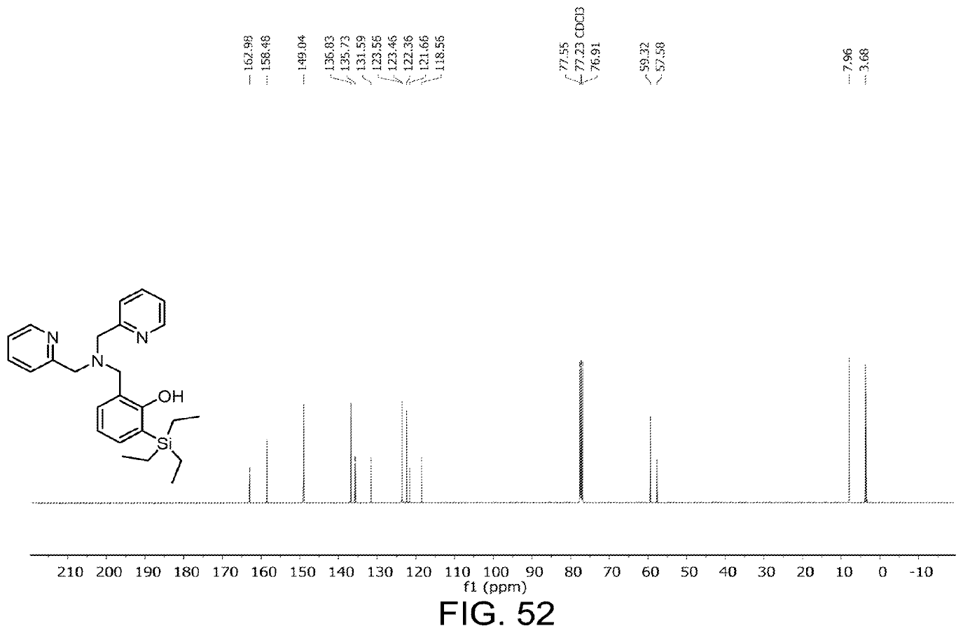

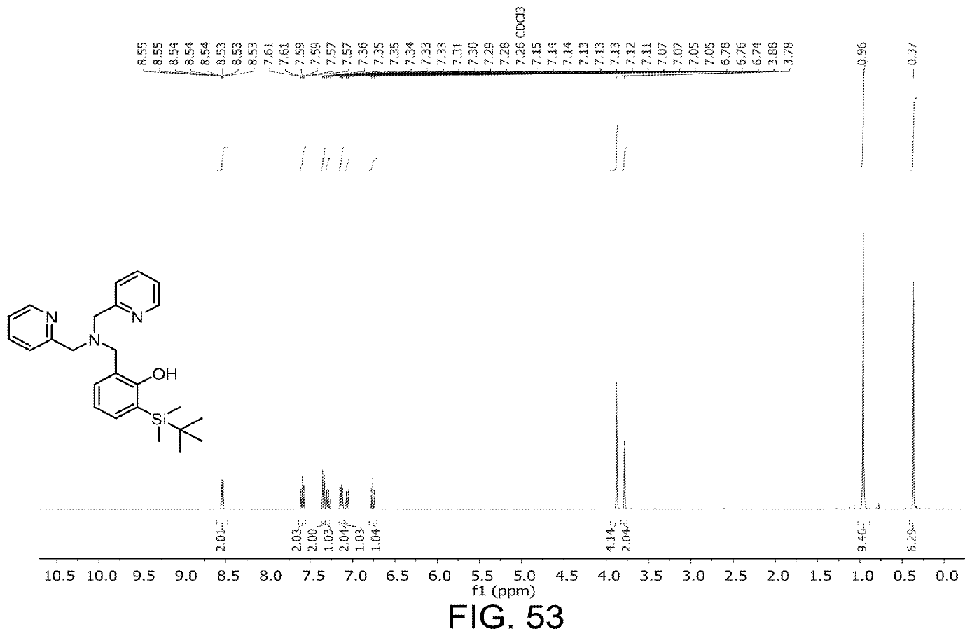

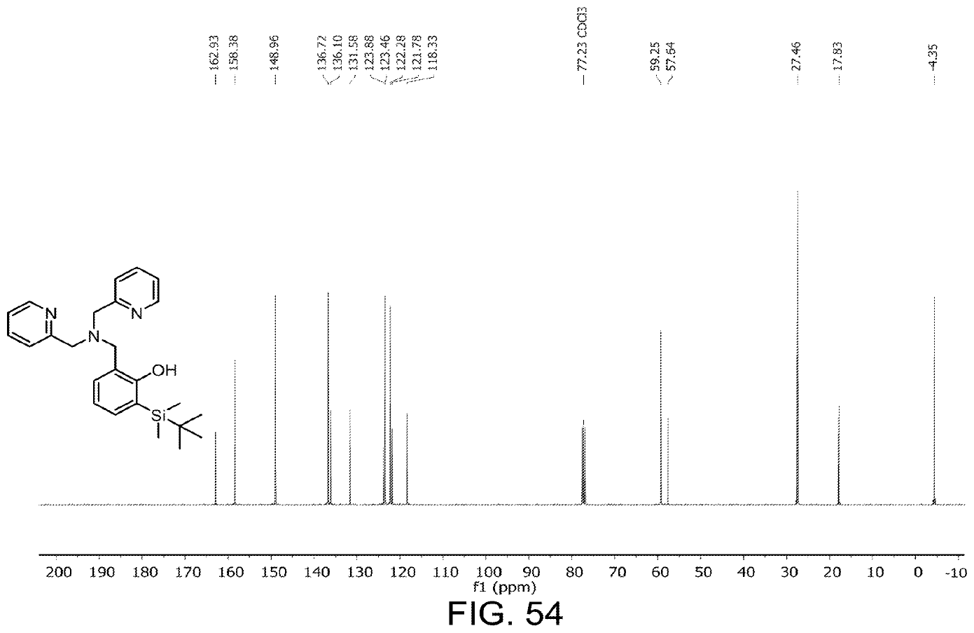

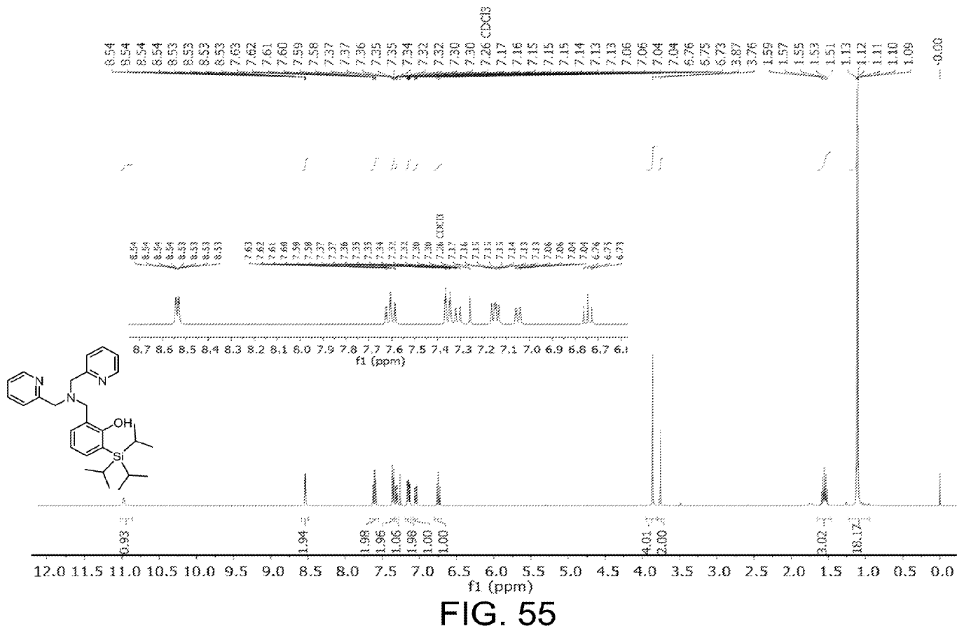

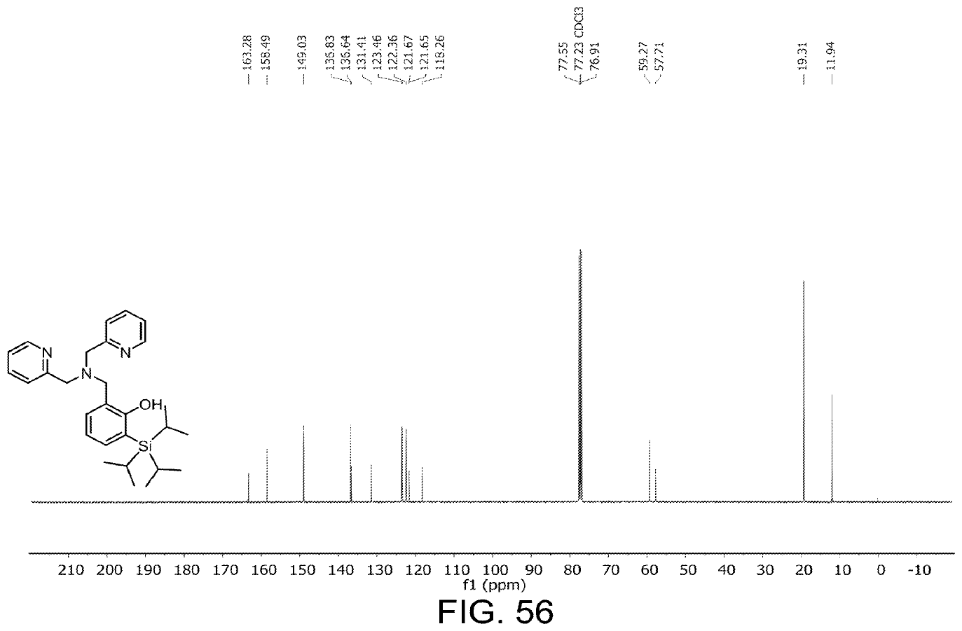

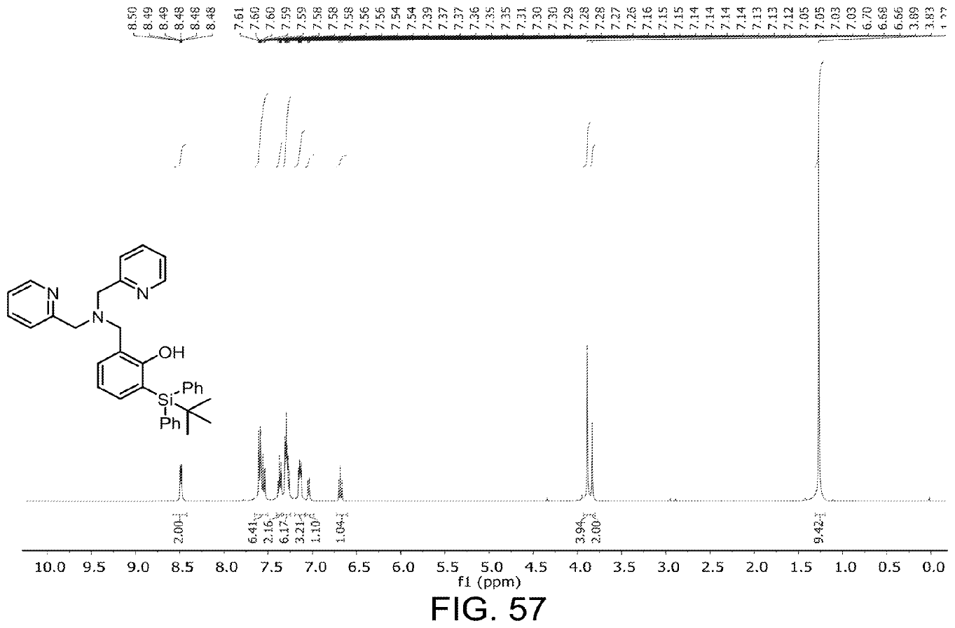

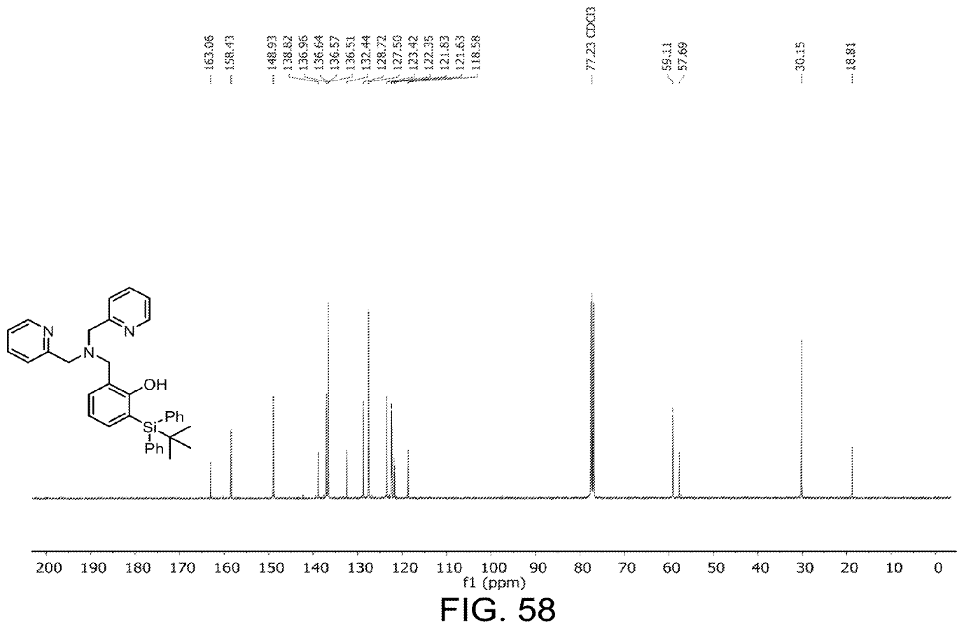

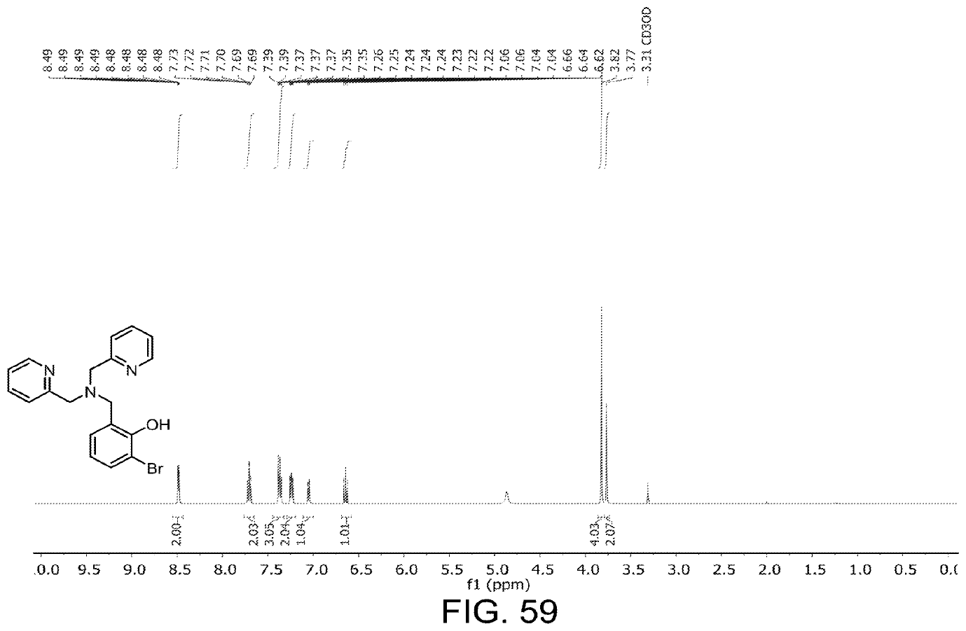

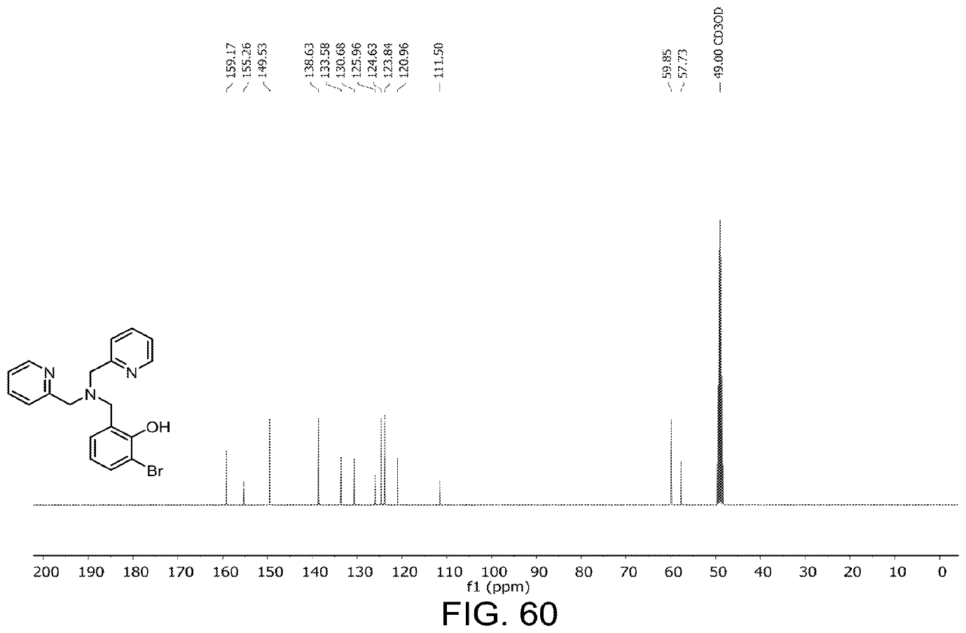

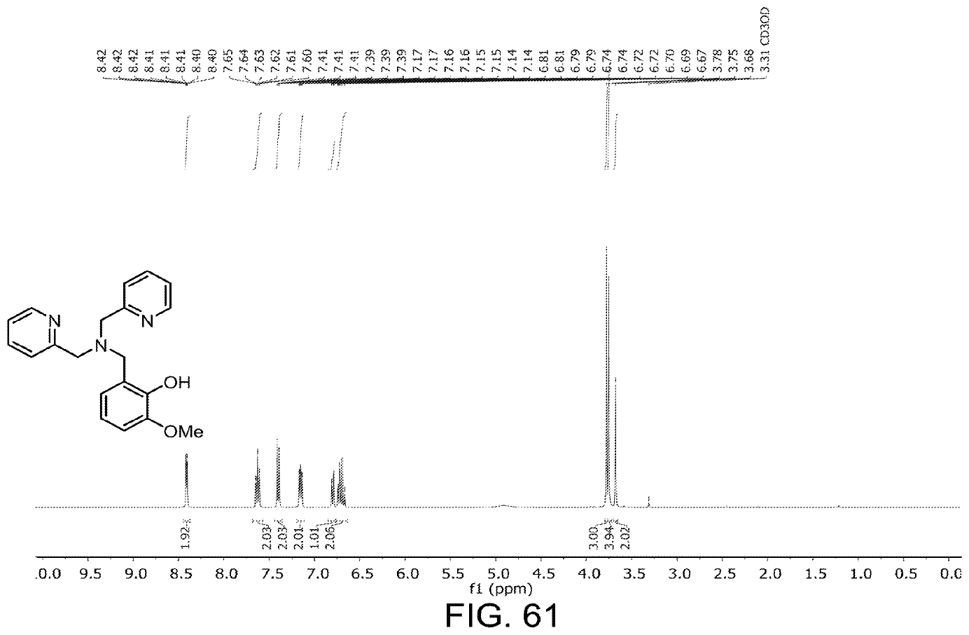

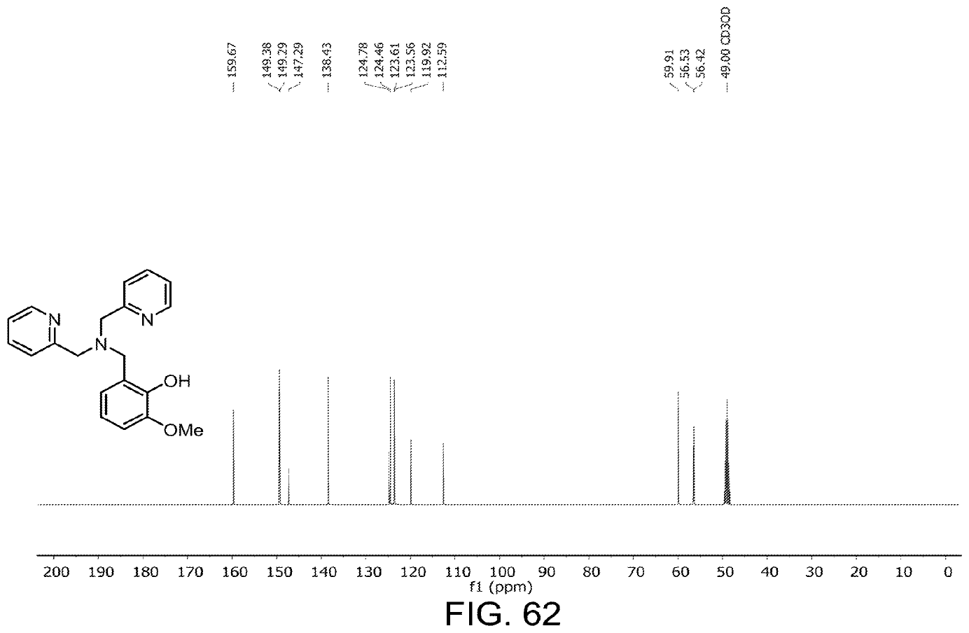

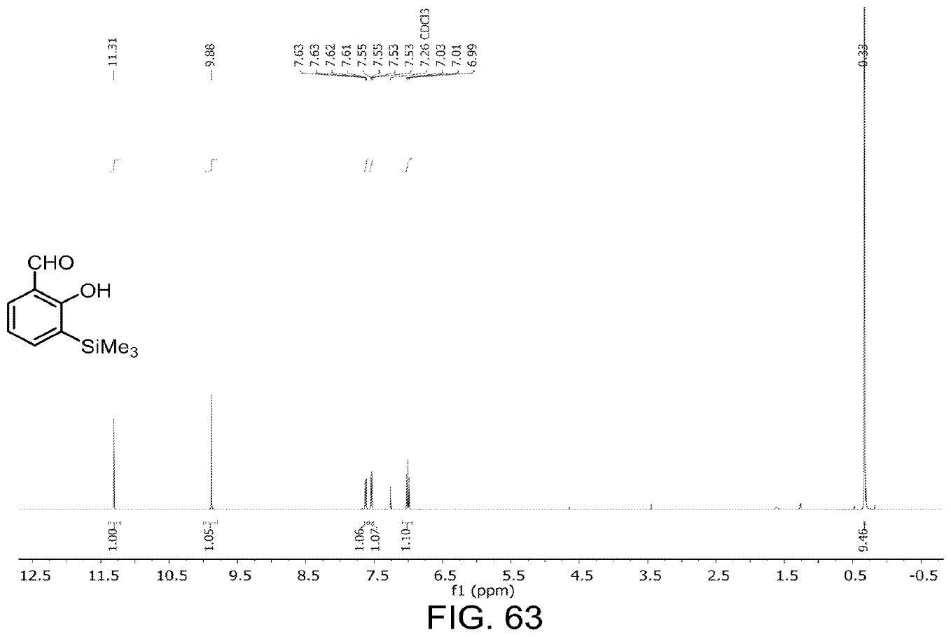

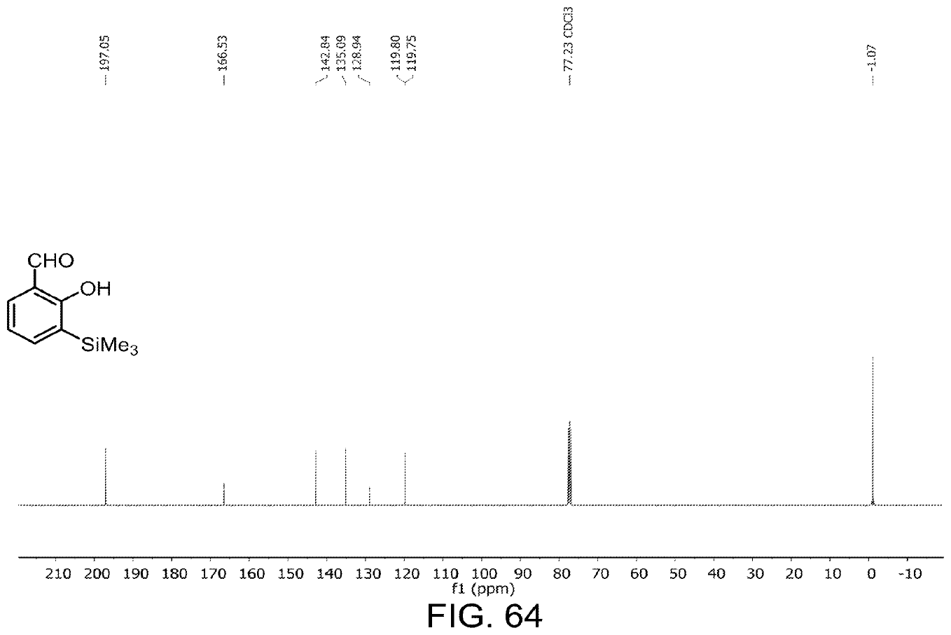

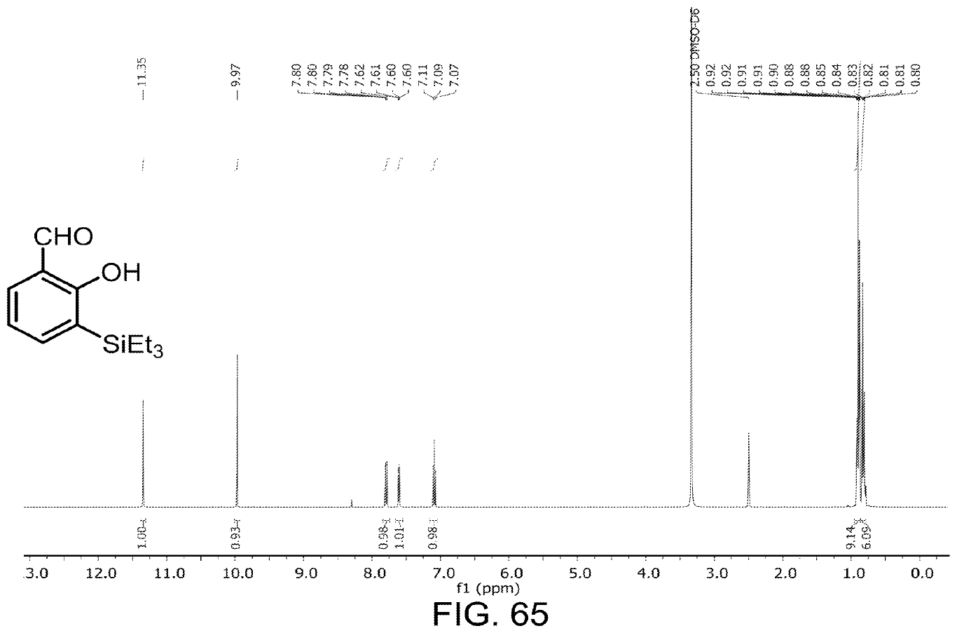

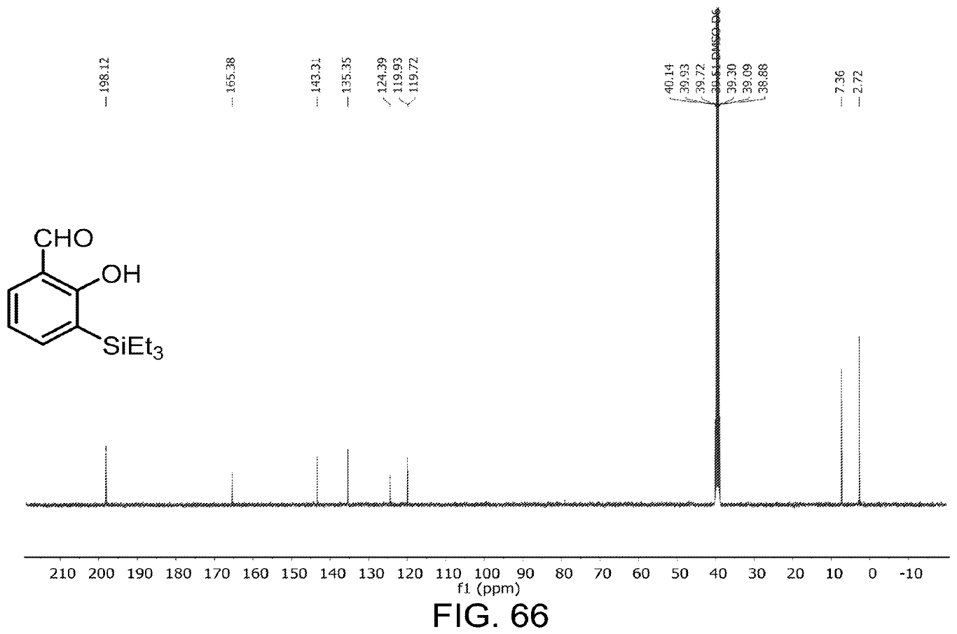

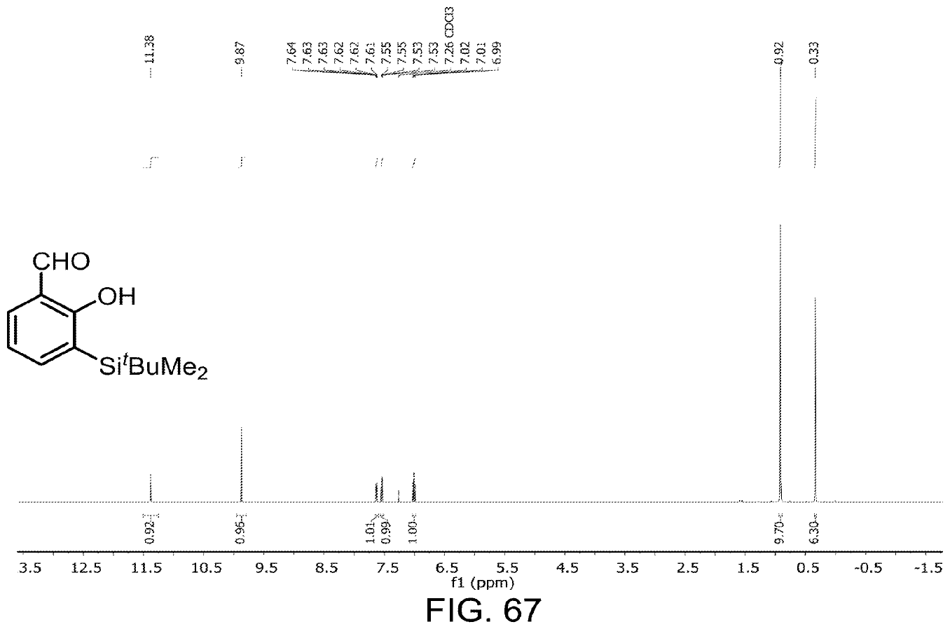

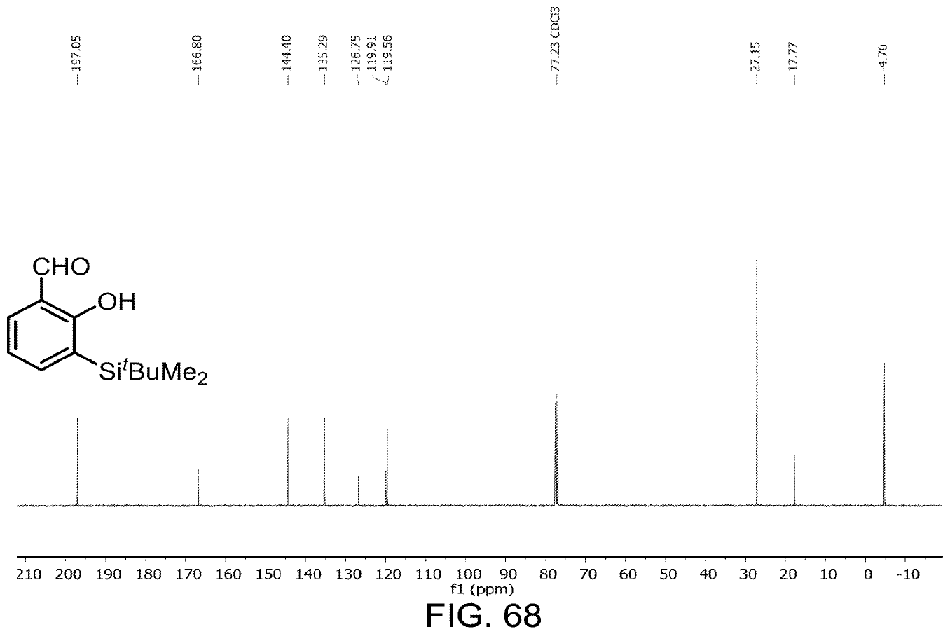

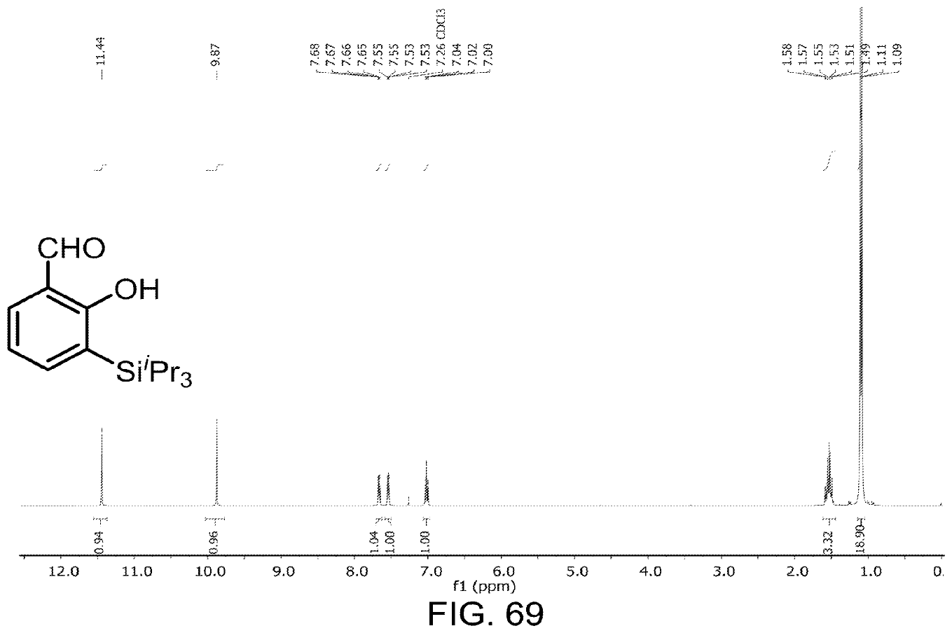

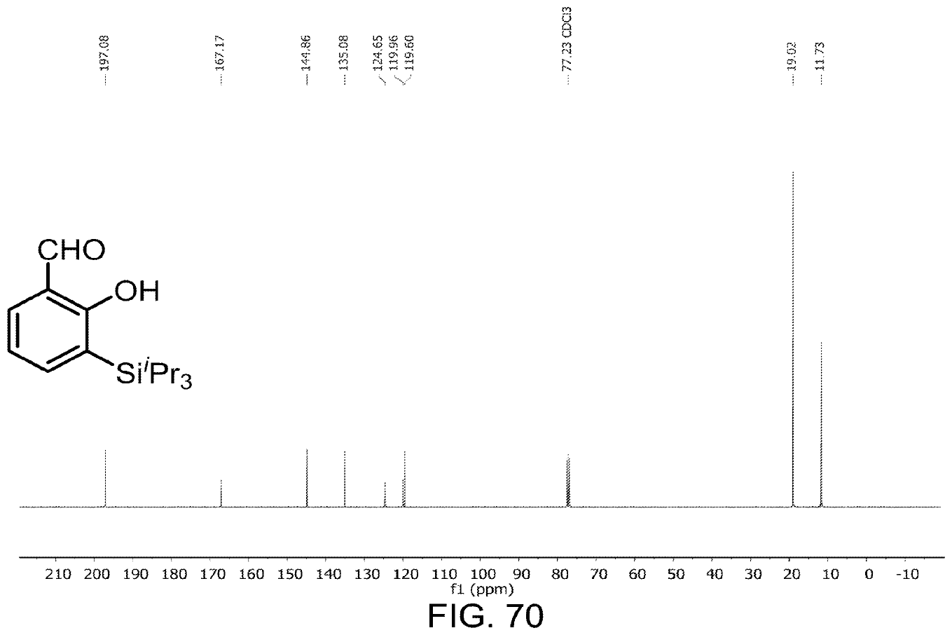

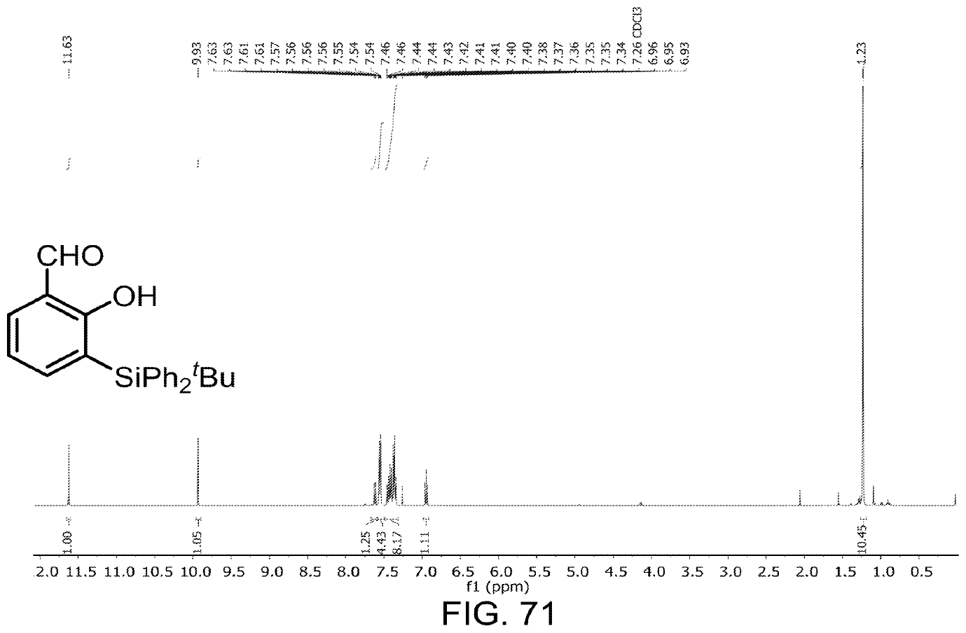

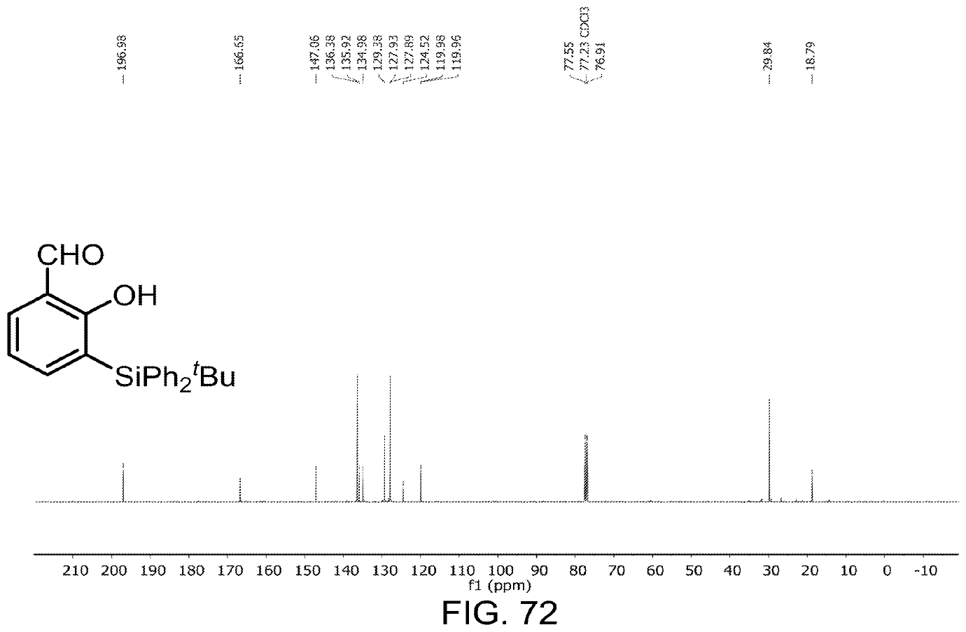

Further supporting data for Example 1 (¹H NMR spectra and 13C NMR spectra) are found in FIGS. 41-72.

### Example 2—Rational Design and Validation of Potent Ionophores for Metal Ions

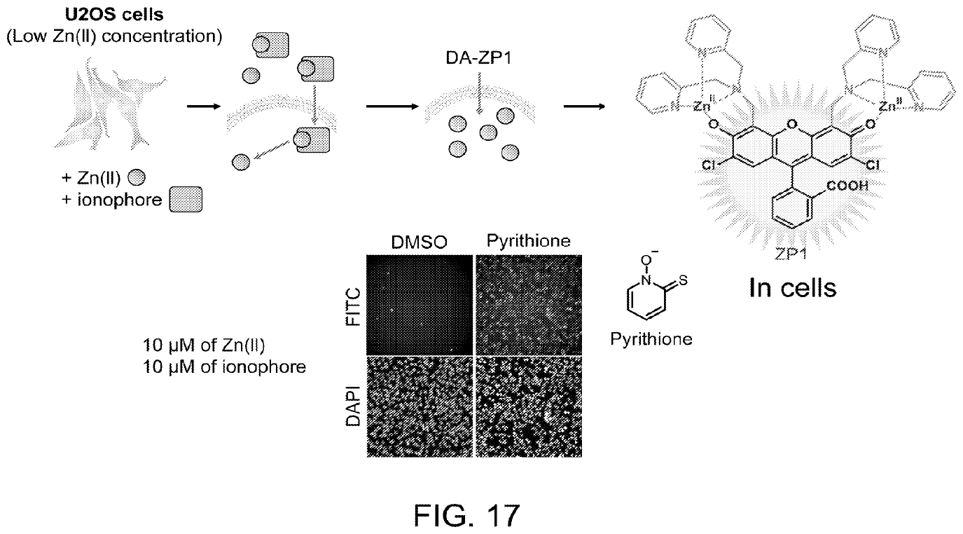

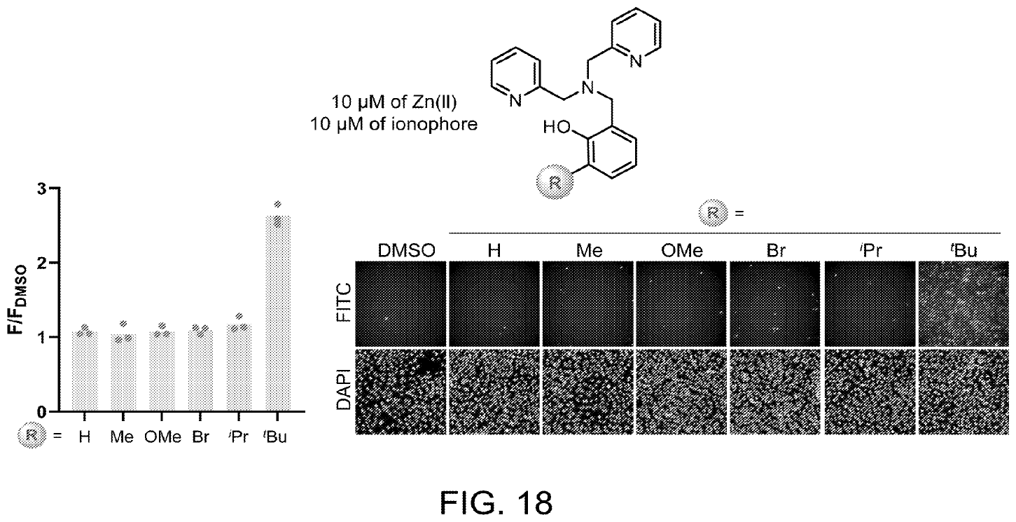

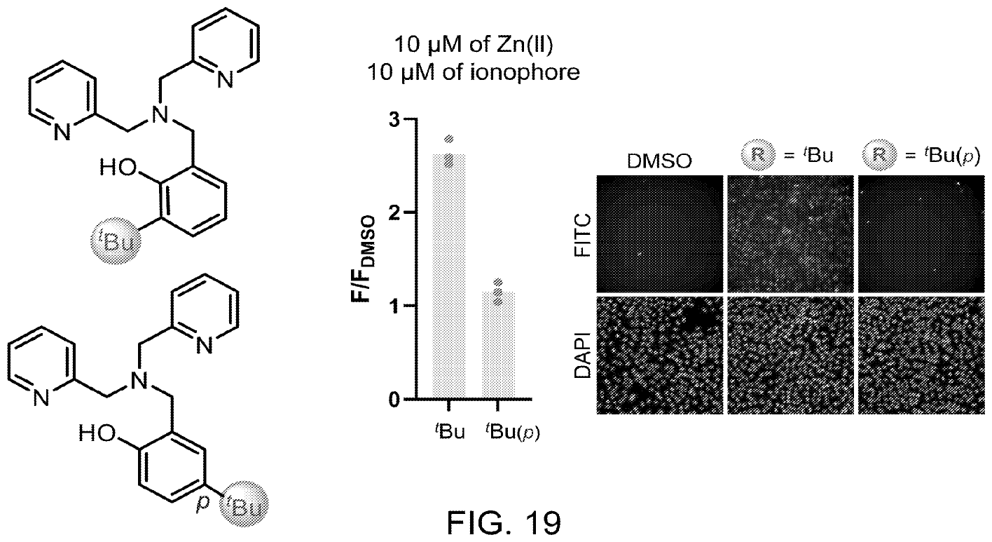

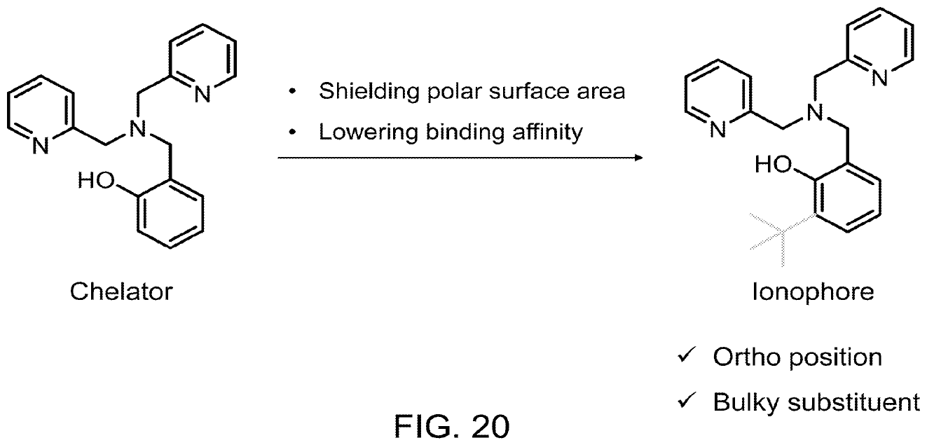

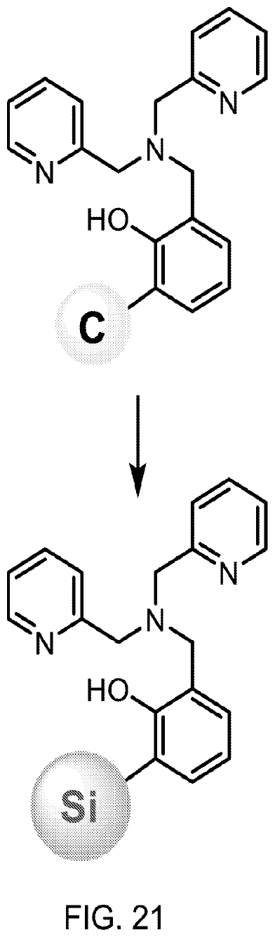

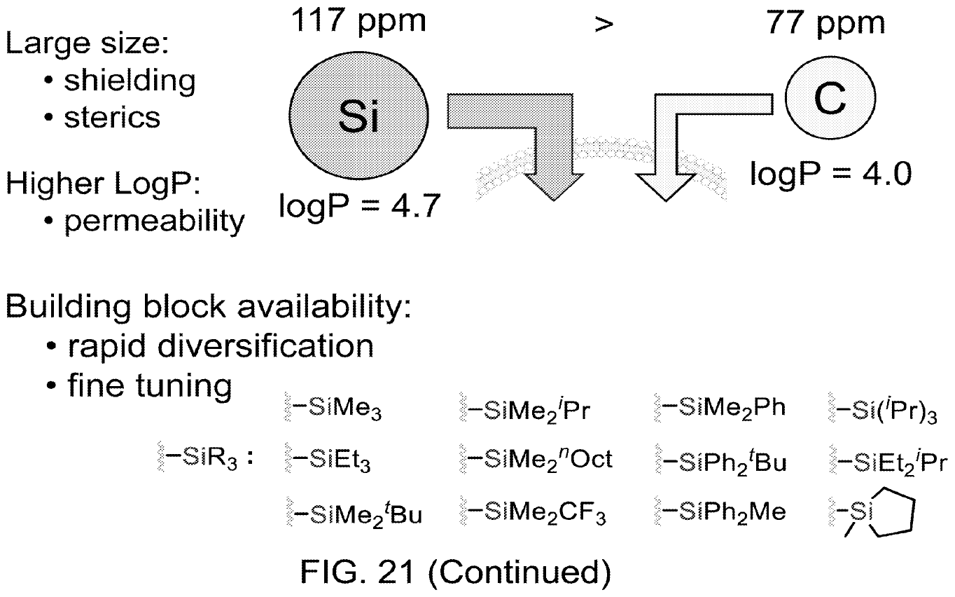

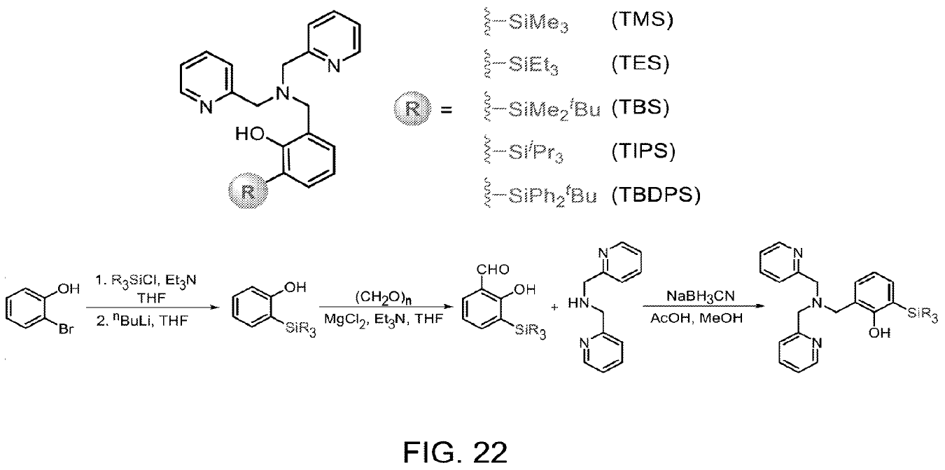

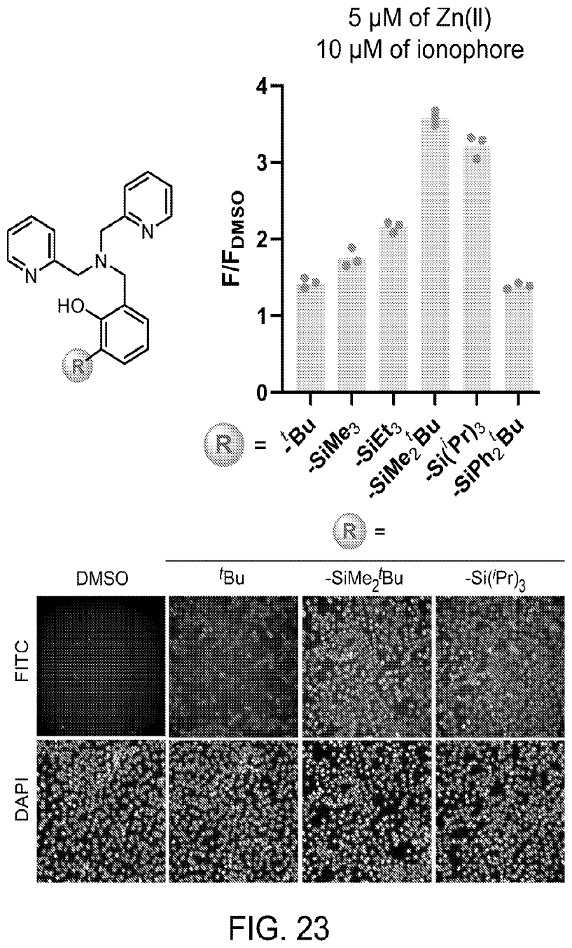

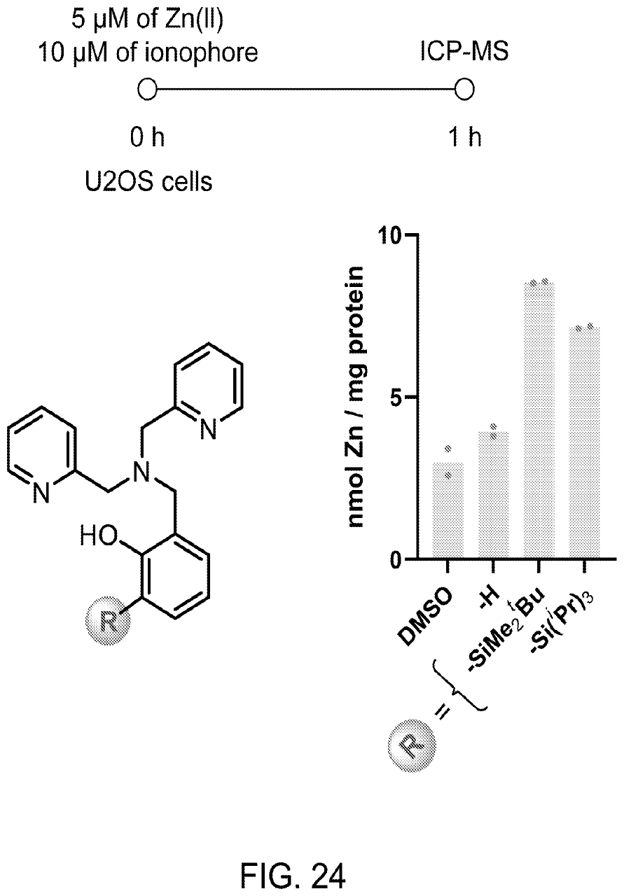

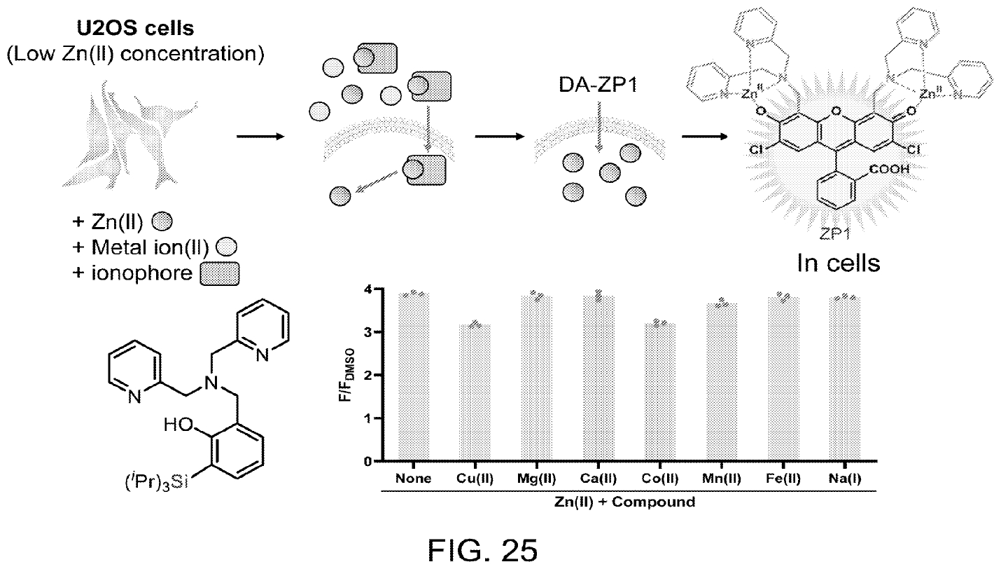

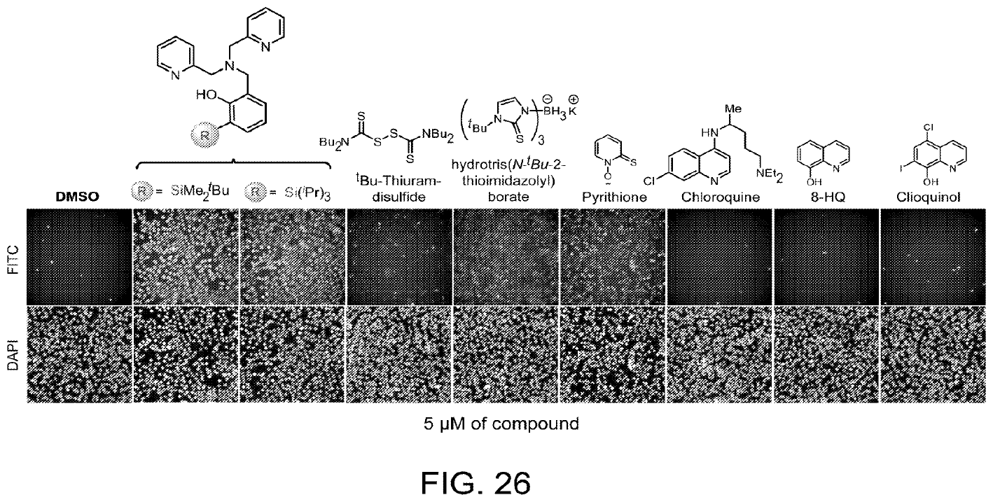

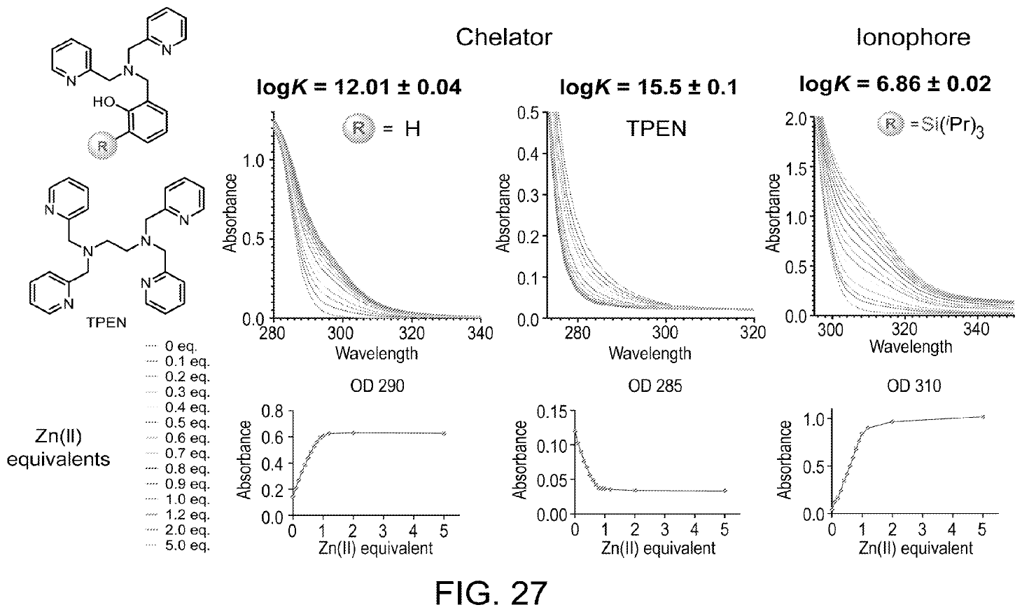

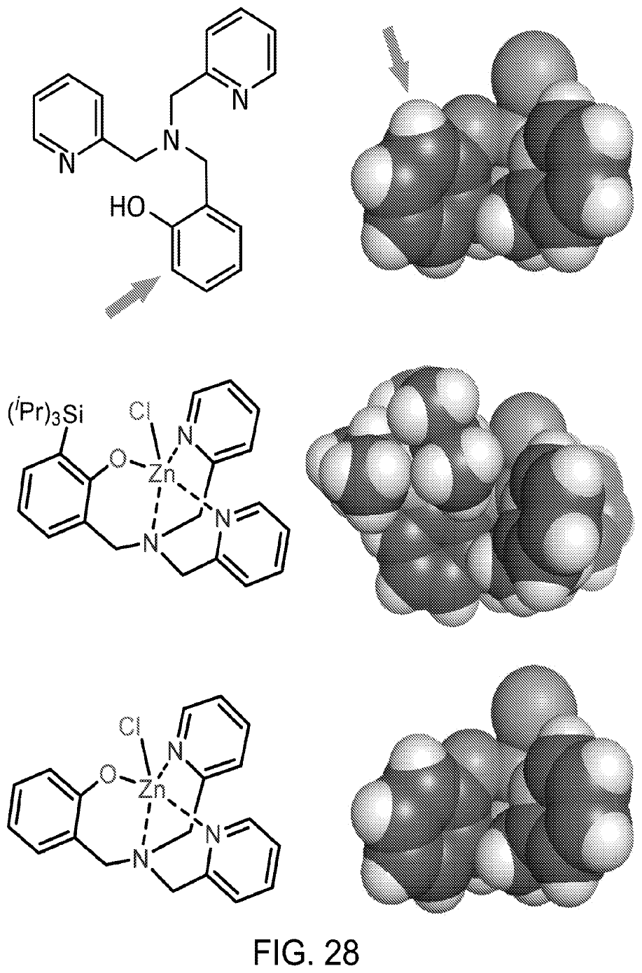

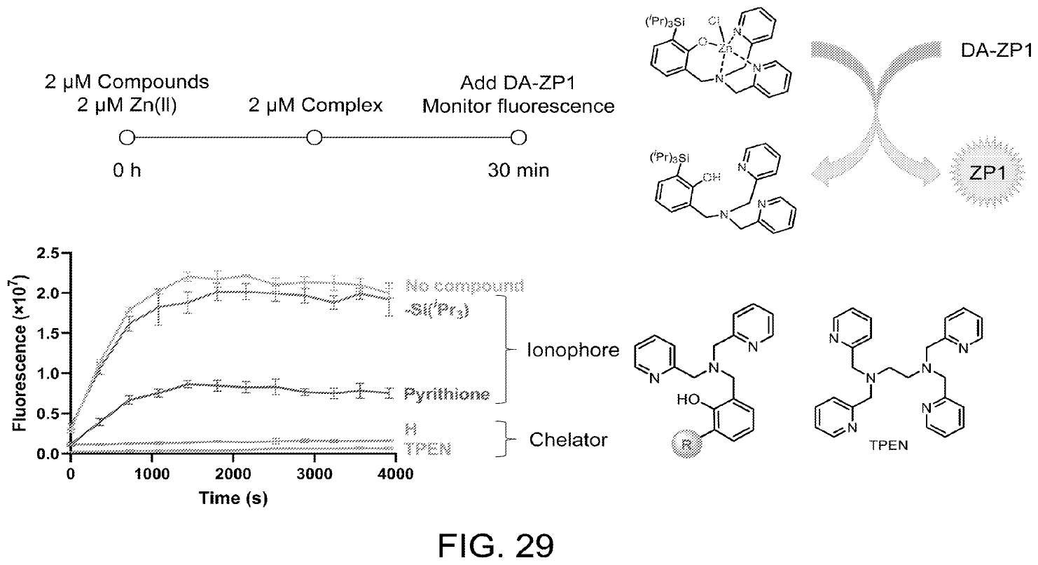

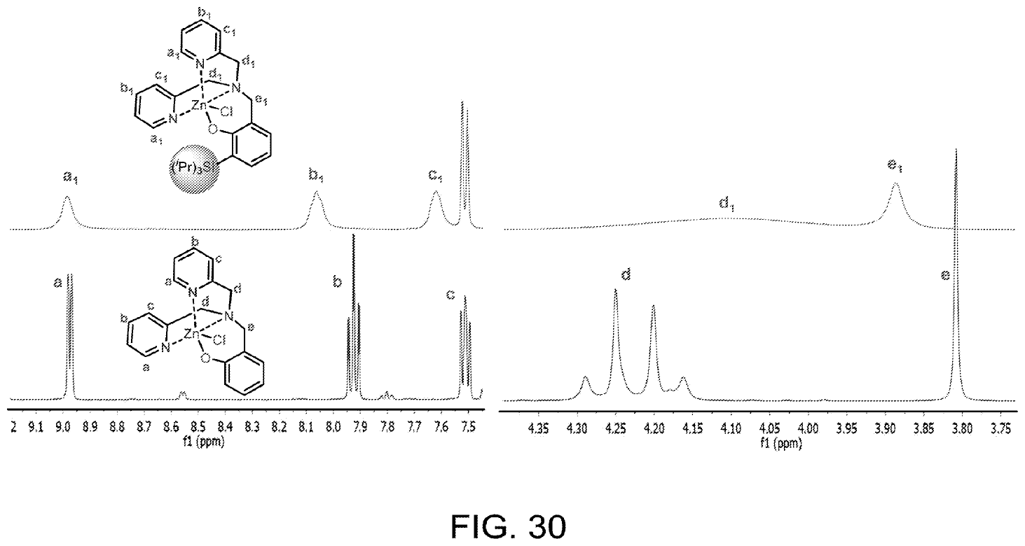

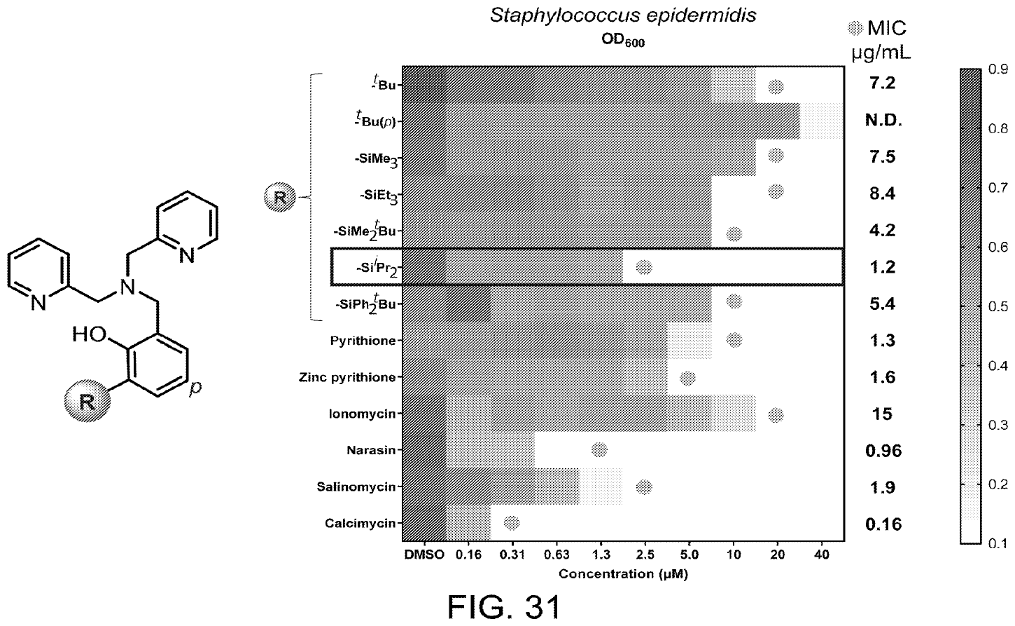

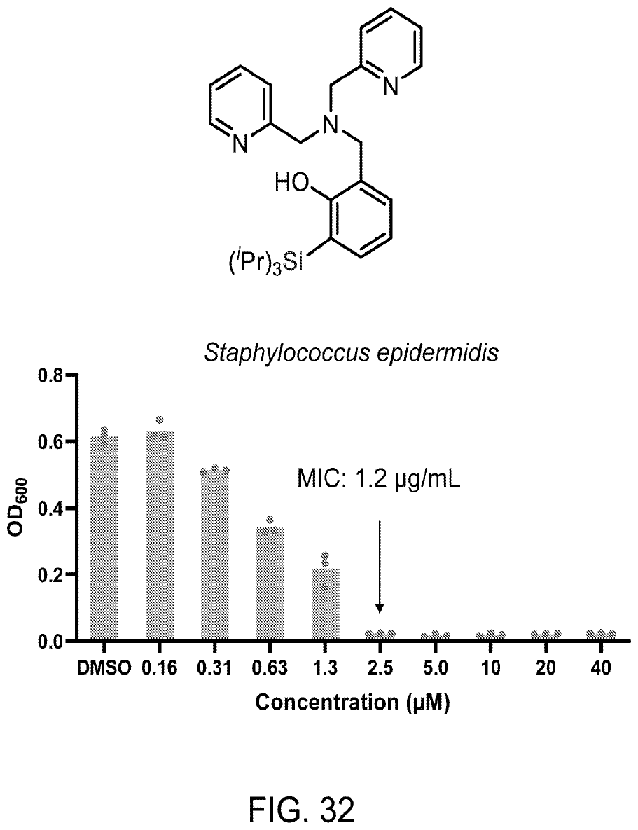

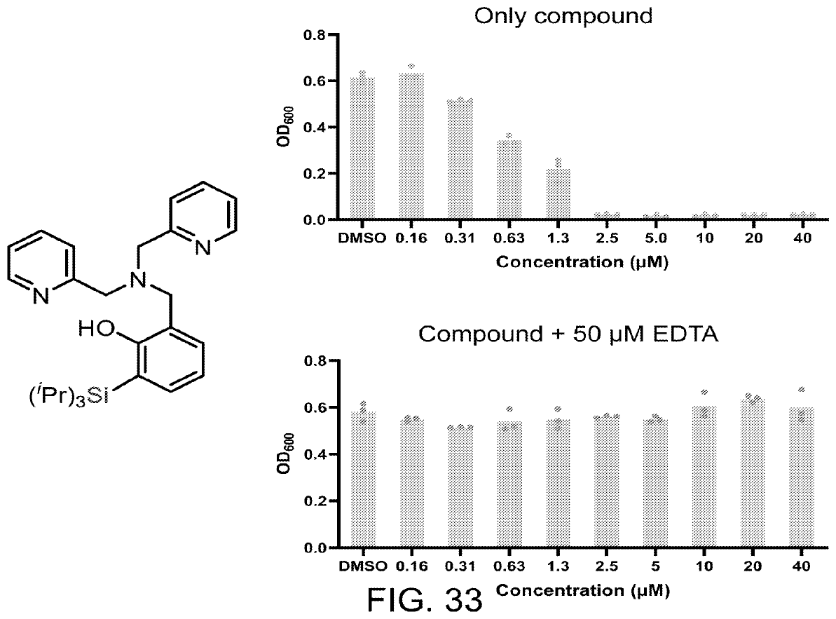

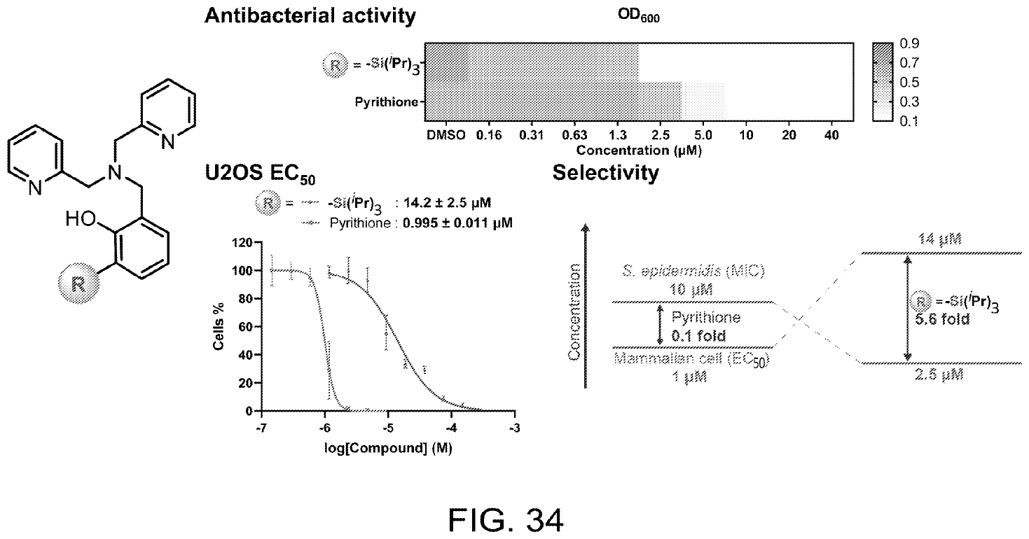

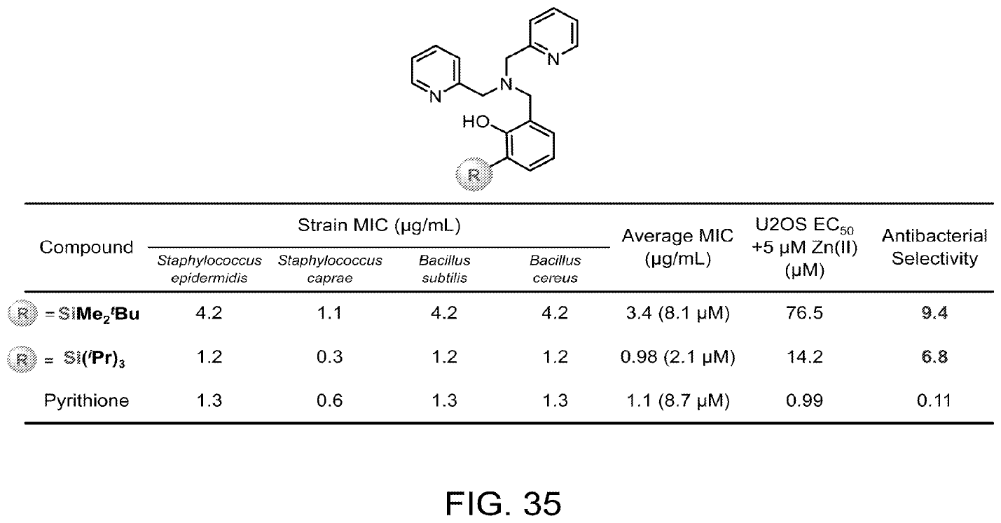

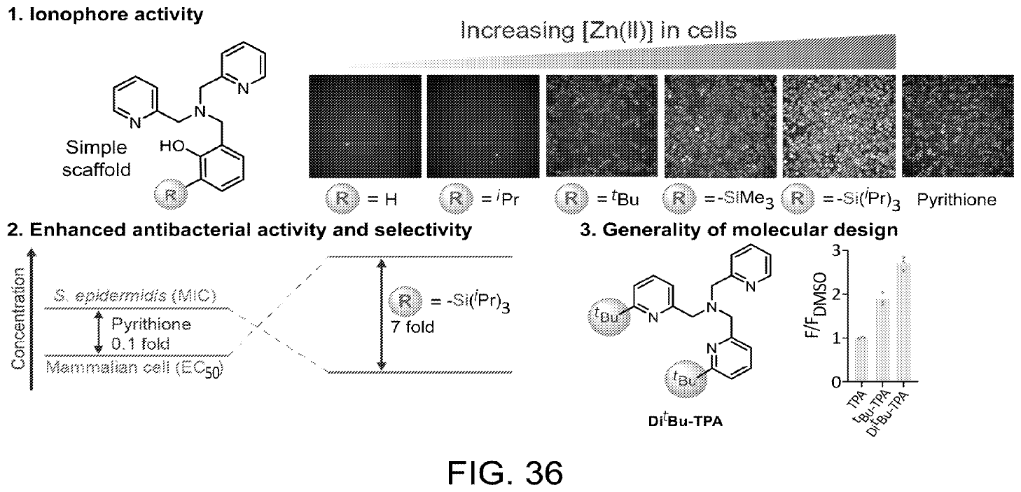

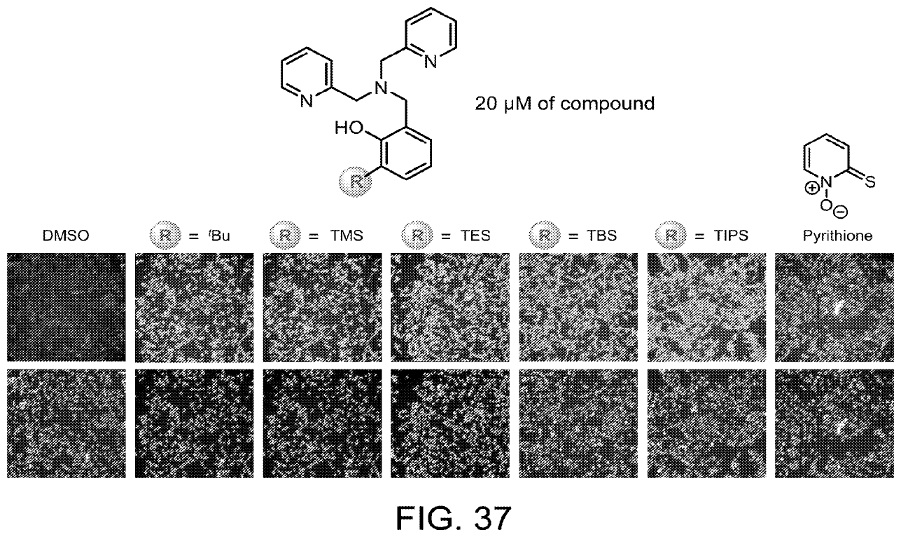

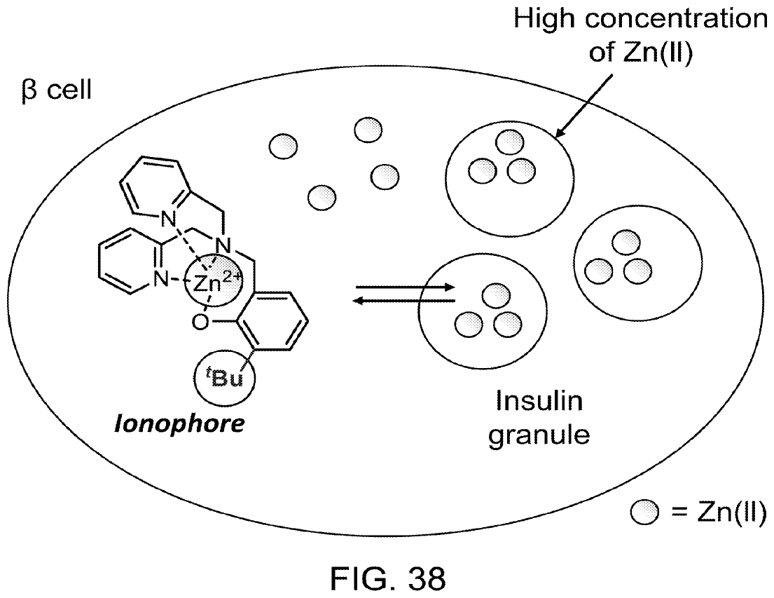

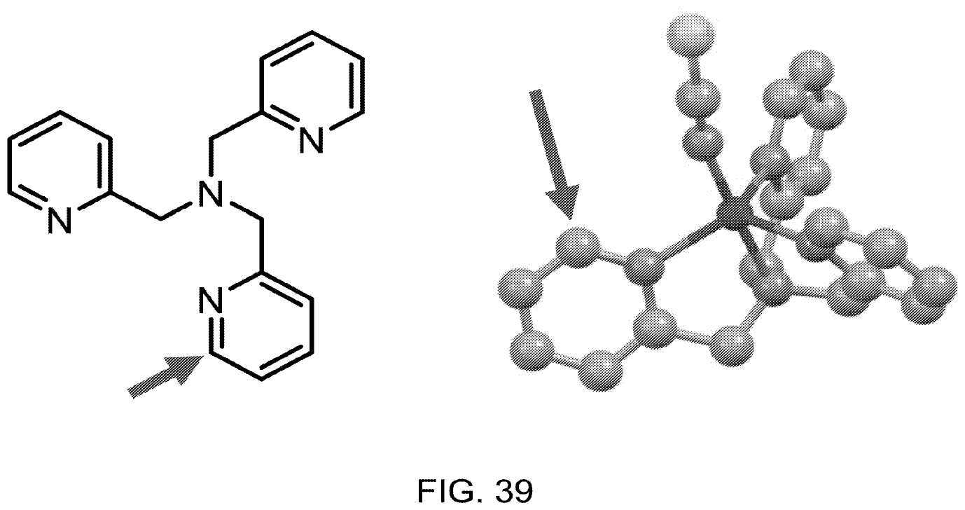

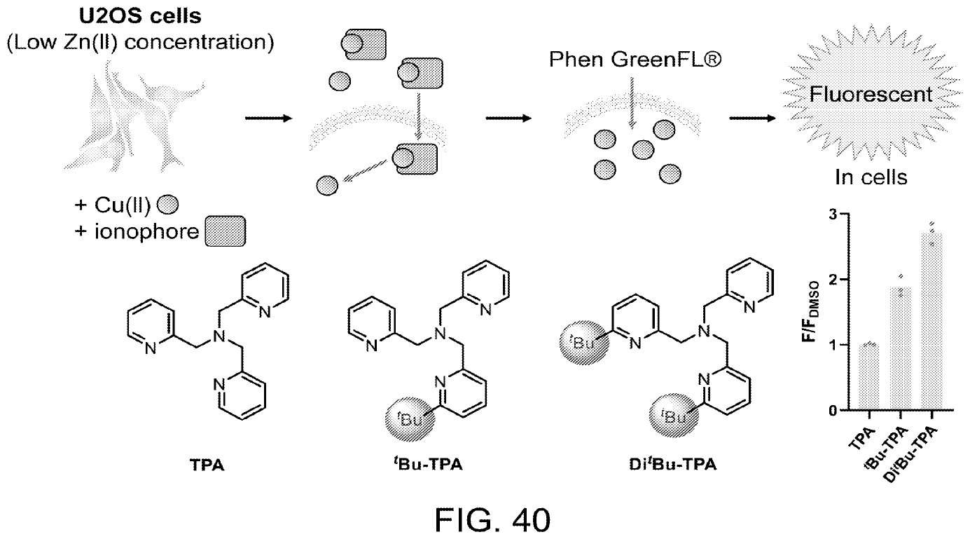

Further details regarding the rational design and validation of potent ionophores for zinc and other metal ions, including, but not limited to, copper ions, are found in FIGS. 17-40.

Embodiments of the present invention may be further defined by reference to the following numbered paragraphs.

1. An engineered ionophore comprising:
a metal ion chelator group comprising a polar binding site, wherein two or more binding atom(s) of the polar binding site are capable of binding a metal ion to produce a chelate complex; and one or more shielding group(s) attached to the metal ion chelator group, wherein each shielding group is in proximity to one or more of the binding atom(s), and wherein the chelate complex exhibits increased hydrophobic membrane permeability and reduced metal ion binding affinity as compared to a counterpart chelate complex in the absence of the shielding group(s).

2. The engineered ionophore of paragraph 1, wherein the metal ion is selected from zinc (Zn), copper (Cu), iron (Fe), gadolinium (Gd), cobalt (Co), lead (Pb), manganese (Mn), lithium (Li), magnesium (Mg), aluminum (Al), calcium (Ca) and silver (Ag) ions.

3. The engineered ionophore of paragraph 1 or 2, wherein the metal ion chelator group is selective for a single metal ion.

4. The engineered ionophore of any one of paragraphs 1-3, wherein the shielding group(s) independently at each occurrence comprise(s) one or more carbon atom(s), one or more silicon atom(s), one or more germanium atom(s), or any combination thereof.

5 The engineered ionophore of any one of paragraphs 1-4, wherein one or more of the shielding group(s) is/are independently at each occurrence selected from alkyl, alkenyl, alkynyl, saturated cyclic hydrocarbon, unsaturated cyclic hydrocarbon, heteroalkyl, heterocyclic ring, aryl ring, and heteroaryl ring group(s), and one or more fused rings thereof, preferably selected from alkyl, heteroalkyl, cycloalkyl, heterocyclic ring, aryl ring, and heteroaryl ring group(s), more preferably selected from C4 or greater alkyl group(s).

6. The engineered ionophore of any one of paragraphs 1-5, wherein one or more of the shielding group(s) is/are independently at each occurrence selected from organosilyl group(s), preferably selected from trialkyl organosilyl groups, alkyldiaryl organosilyl groups, dialkylaryl organosilyl groups, and triaryl organosilyl groups, more preferably, wherein each alkyl group is independently at each occurrence selected from C₁-C₆ alkyl groups.

7. The engineered ionophore of any one of paragraphs 1-6, wherein one or more of the shielding group(s) is/are independently at each occurrence selected from organogermanyl group(s), preferably selected from trialkyl or triphenyl organogermanyl groups, more preferably, wherein each alkyl group is independently at each occurrence selected from C₁-C₆ alkyl groups.

8. The engineered ionophore of any one of paragraphs 1-7, wherein one or more of the shielding group(s) is/are independently at each occurrence in an ortho-position to one of the binding atom(s).

9. The engineered ionophore of paragraph 1, wherein the metal ion chelator group and/or the metal ion is/are selected from Table 1, wherein the shielding group(s) is/are independently at each occurrence selected from Table 2, and/or wherein the metal ion chelator group and/or the shielding group(s) is/are independently at each occurrence selected from Table 3.

10. A pharmaceutical composition comprising at least one engineered ionophore of any one of paragraphs 1-9, and, optionally, at least metal ion capable of binding with the at least one engineered ionophore.

11. A kit comprising at least one engineered ionophore of any one of paragraphs 1-9, and, optionally, at least one metal ion capable of binding with the at least one engineered ionophore.

12. An ion-selective membrane device comprising at least one hydrophobic membrane, at least one engineered ionophore of any one of paragraphs 1-9, and at least one metal ion capable of binding with the at least one engineered ionophore.

13. The ion-selective membrane device of paragraph 12, wherein the ion-selective membrane device is selected from ion-selective membrane electrodes or ion-selective membrane sensors.

14. A method of increasing hydrophobic membrane transport of metal ions, the method comprising:
forming a reaction mixture comprising:
   a metal ion; and
   an engineered ionophore of any one of paragraphs 1-9,
   wherein a chelate complex is formed; and
contacting the reaction mixture with a hydrophobic membrane, wherein the chelate complex is transported across the hydrophobic membrane and the metal ion is released from the transported chelate complex, and wherein the chelate complex expresses increased hydrophobic membrane transport and/or metal ion release as compared to a counterpart chelate complex in the absence of the shielding group(s).

15. The method of paragraph 14, wherein the hydrophobic membrane is a cellular membrane, and wherein:
when the reaction mixture is formed and contacted with the cellular membrane outside of a cell, a vesicle, or a cellular organelle, the chelate complex is transported across the cellular membrane into the cell, the vesicle, or the cellular organelle; and
when the reaction mixture is formed and contacted with the cellular membrane inside of a cell, a vesicle, or a cellular organelle, the chelate complex is transported across the cellular membrane and out of the cell, the vesicle, or the cellular organelle.

16. The method of paragraph 15, wherein the contacting the reaction mixture with a cellular membrane occurs in a human or a part thereof, or a non-human animal, a plant, or a part thereof, and wherein the method targets, diagnoses, treats, prevents, or any combination thereof, a current or potential disease, disease state, condition, disorder, side effect, or the like, or any combination thereof, of the human or the part thereof, or the non-human animal, the plant, or the part thereof.

17. The method of paragraph 16, wherein the current or potential disease, disease state, condition, disorder, side effect, or the like, or any combination thereof, is selected from a metal ion deficiency or overload, a microbial infection, cancer, chronic kidney disease, Alzheimer's disease, ALS, Wilson disease, diabetes, and sickle cell anemia.

18. The method of paragraph 16, wherein the current or potential disease, disease state, condition, disorder, side effect, or the like, or any combination thereof, is a microbial infection, and wherein the method increases antimicrobial activity against the infection in the human or the part thereof, or the non-human animal, the plant, or the part thereof.

19. The method of any one of paragraphs 16-18, wherein the method further comprises, prior to, concurrently with, or after forming the reaction mixture, administering the pharmaceutical composition of paragraph 9 to the human or the part thereof, or the non-human animal, the plant, or the part thereof.

20. The method of paragraph 14, wherein the method occurs within the ion-selective membrane device of paragraph 11 or 12.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present invention come within known customary practice within the art to which the invention pertains and may be applied to the essential features herein before set forth.

What is claimed is:

1. An engineered ionophore comprising:
a metal ion chelator group comprising a polar binding site, wherein two or more binding atom(s) of the polar binding site are capable of binding a metal ion to produce a chelate complex; and
one or more shielding group(s) attached to the metal ion chelator group, wherein each shielding group is in proximity to one or more of the binding atom(s), and wherein the chelate complex exhibits increased hydrophobic membrane permeability and reduced metal ion binding affinity as compared to a counterpart chelate complex in the absence of the shielding group(s).

2. The engineered ionophore of claim 1, wherein the metal ion is selected from zinc (Zn), copper (Cu), iron (Fe), gadolinium (Gd), cobalt (Co), lead (Pb), manganese (Mn), lithium (Li), magnesium (Mg), aluminum (Al), calcium (Ca) and silver (Ag) ions.

3. The engineered ionophore of claim 1 or 2, wherein the metal ion chelator group is selective for a single metal ion.

4. The engineered ionophore of any one of claims 1-3, wherein the shielding group(s) independently at each occurrence comprise(s) one or more carbon atom(s), one or more silicon atom(s), one or more germanium atom(s), or any combination thereof.

5. The engineered ionophore of any one of claims 1-4, wherein one or more of the shielding group(s) is/are independently at each occurrence selected from alkyl, alkenyl, alkynyl, saturated cyclic hydrocarbon, unsaturated cyclic hydrocarbon, heteroalkyl, heterocyclic ring, aryl ring, and heteroaryl ring group(s), and one or more fused rings thereof, preferably selected from alkyl, heteroalkyl, cycloalkyl, heterocyclic ring, aryl ring, and heteroaryl ring group(s), more preferably selected from C₄ or greater alkyl group(s).

6. The engineered ionophore of any one of claims 1-5, wherein one or more of the shielding group(s) is/are independently at each occurrence selected from organosilyl group(s), preferably selected from trialkyl organosilyl groups, alkyldiaryl organosilyl groups, dialkylaryl organosilyl groups, and triaryl organosilyl groups, more preferably, wherein each alkyl group is independently at each occurrence selected from C₁-C₆ alkyl groups.

7. The engineered ionophore of any one of claims 1-6, wherein one or more of the shielding group(s) is/are independently at each occurrence selected from organogermanyl group(s), preferably selected from trialkyl or triphenyl organogermanyl groups, more preferably, wherein each alkyl group is independently at each occurrence selected from C₁-C₆ alkyl groups.

8. The engineered ionophore of any one of claims 1-7, wherein one or more of the shielding group(s) is/are independently at each occurrence in an ortho-position to one of the binding atom(s).

9. The engineered ionophore of claim 1, wherein the metal ion chelator group and/or the metal ion is/are selected from Table 1, wherein the shielding group(s) is/are independently at each occurrence selected from Table 2, and/or wherein the metal ion chelator group and/or the shielding group(s) is/are independently at each occurrence selected from Table 3.

10. A pharmaceutical composition comprising at least one engineered ionophore of any one of claims 1-9, and, optionally, at least metal ion capable of binding with the at least one engineered ionophore.

11. A kit comprising at least one engineered ionophore of any one of claims 1-9, and, optionally, at least one metal ion capable of binding with the at least one engineered ionophore.

12. An ion-selective membrane device comprising at least one hydrophobic membrane, at least one engineered ionophore of any one of claims 1-9, and at least one metal ion capable of binding with the at least one engineered ionophore.

13. The ion-selective membrane device of claim 12, wherein the ion-selective membrane device is selected from ion-selective membrane electrodes or ion-selective membrane sensors.

14. A method of increasing hydrophobic membrane transport of metal ions, the method comprising:
forming a reaction mixture comprising:
a metal ion; and
an engineered ionophore of any one of claims 1-9,
wherein a chelate complex is formed; and
contacting the reaction mixture with a hydrophobic membrane, wherein the chelate complex is transported across the hydrophobic membrane and the metal ion is released from the transported chelate complex, and wherein the chelate complex expresses increased hydrophobic membrane transport and/or metal ion release as compared to a counterpart chelate complex in the absence of the shielding group(s).

15. The method of claim 14, wherein the hydrophobic membrane is a cellular membrane, and wherein:
when the reaction mixture is formed and contacted with the cellular membrane outside of a cell, a vesicle, or a cellular organelle, the chelate complex is transported across the cellular membrane into the cell, the vesicle, or the cellular organelle; and
when the reaction mixture is formed and contacted with the cellular membrane inside of a cell, a vesicle, or a cellular organelle, the chelate complex is transported across the cellular membrane and out of the cell, the vesicle, or the cellular organelle.

16. The method of claim 15, wherein the contacting the reaction mixture with a cellular membrane occurs in a human or a part thereof, or a non-human animal, a plant, or a part thereof, and wherein the method targets, diagnoses, treats, prevents, or any combination thereof, a current or potential disease, disease state, condition, disorder, side effect, or the like, or any combination thereof, of the human or the part thereof, or the non-human animal, the plant, or the part thereof.

17. The method of claim 16, wherein the current or potential disease, disease state, condition, disorder, side effect, or the like, or any combination thereof, is selected from a metal ion deficiency or overload, a microbial infection, cancer, chronic kidney disease, Alzheimer's disease, ALS, Wilson disease, diabetes, and sickle cell anemia.

18. The method of claim 16, wherein the current or potential disease, disease state, condition, disorder, side effect, or the like, or any combination thereof, is a microbial infection, and wherein the method increases antimicrobial activity against the infection in the human or the part thereof, or the non-human animal, the plant, or the part thereof.

19. The method of any one of claims 16-18, wherein the method further comprises, prior to, concurrently with, or after forming the reaction mixture, administering the pharmaceutical composition of claim 9 to the human or the part thereof, or the non-human animal, the plant, or the part thereof.

20. The method of claim 14, wherein the method occurs within the ion-selective membrane device of claim 11 or 12.
